(19) United States
(12) Patent Application Publication
(10) Pub. No.: US 2011/0059910 A1
(43) Pub. Date: Mar. 10, 2011

(54) Novel aromatic fluoroglycoside derivatives, pharmaceuticals comprising said compounds and the use thereof

(75) Inventors: Wendelin FRICK, Hunstetten-Beuerbach (DE); Heiner GLOMBIK, Hofheim (DE); Stefan THEIS, Frankfurt (DE); Ralf ELVERT, Bad Hersfeld (DE)

(73) Assignee: SANOFI-AVENTIS, Paris (FR)

(21) Appl. No.: 12/851,944

(22) Filed: Aug. 6, 2010

Related U.S. Application Data
(63) Continuation of application No. PCT/EP2009/001042, filed on Feb. 13, 2009.

(30) Foreign Application Priority Data
Feb. 13, 2008 (EP) 08290152.1

(51) Int. Cl. A61K 31/7034; A61K 31/70; A61K 31/706; C07H 15/20; C07H 15/26; C07H 7/06; C07H 11/00; C07H 5/02; A61P 3/10

(52) U.S. Cl. 514/25; 514/23; 536/18.1; 536/18.2; 536/18.4; 536/18.7; 536/115; 536/122

(57) ABSTRACT

The invention relates to substituted aromatic fluoroglycoside derivatives, and to the physiologically compatible salts and physiologically functional derivatives thereof. The invention also relates to methods of lowering blood sugar and the treatment of type I and type II diabetes.

The invention relates to substituted aromatic fluoroglycoside derivatives, and to the physiologically compatible salts and physiologically functional derivatives thereof.

Several substance classes with SGLT action have already been disclosed in the literature. The model for all of these structures was the natural product phlorizine. From this were derived the following classes which are described in the following property rights:
propiophenone glycosides of Tanabe (WO 0280936, WO 0280935, JP 2000080041 and EP 850948)
2-(glucopyranosyloxy)benzylbenzenes of Kissei (WO 0244192, WO 0228872, WO 03011880 and WO 0168660)
glucopyranosyloxypyrazoles of Kissei, Bristol-Myers Squibb and Ajinomoto (WO 02068440, WO 02068439, WO 0236602, WO 01016147, WO 02053573, WO 03020737, WO 03090783, WO 04014932, WO 04019958 and WO 04018491)
O-glycoside benzamides of Bristol-Myers Squibb (WO 0174835 and WO 0174834)
glucopyranosyloxythiophenes of Aventis (WO 04007517)
C-aryl glycosides of Bristol-Myers Squibb (WO 03099836, WO 0127128 and US 2002137903)
substituted C-aryl glycosides of Boehringer Ingelheim (US2006/0074031)
4-fluorodeoxyglucopyranosides and C-aryl glycosides of Sanofi-Aventis (WO 2004/052902, WO 2004/052903 and WO 2005/121161)
substituted C-aryl glycosides of Mitsubishi Tanabe (WO 2008/013321).

All known structures contain glucose as a very important structural element.

It was an object of the invention to provide novel compounds with which prevention and treatment of type 1 and type 2 diabetes is possible. We have now found that, surprisingly, aromatic fluoroglycoside derivatives selectively enhance the action on SGLT2. These compounds are therefore particularly suitable for prevention and treatment of type 1 and type 2 diabetes.

The invention therefore relates to compounds of the formula I

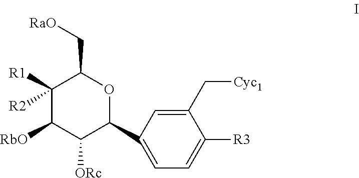

in which
Ra, Rb, Rc are each independently H, —COO—(C₁-C₆)-alkyl;
R1 and R2 are each F or
R1 is H and R2 is F;
R3 is hydrogen, F, Cl, Br, CF₃, OCF₃, CN, methyl, ethyl, methoxy, ethoxy, cyclopropyl, CH₂-cyclopropyl;
Cyc1 is

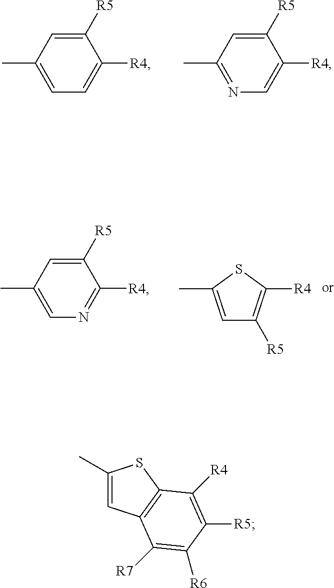

R4, R5, R6, R7 are each independently hydrogen, F, Cl, Br, I, OH, CF₃, NO₂, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, O—(C₁-C₆)-alkyl, HO—(C₁-C₆)-alkylene, (C₁-C₆)-alkylene-O—(C₁-C₆)-alkyl, where one, more than one or all hydrogen(s) in the alkyl, alkenyl, alkynyl or O-alkyl radicals may be replaced by fluorine;
   SO₂—NH₂, SO₂NH(C₁-C₆)-alkyl, SO₂N[(C₁-C₆)-alkyl]₂, S—(C₁-C₆)-alkyl, SCF₃, SO—(C₁-C₆)-alkyl, SO₂—(C₁-C₆)-alkyl, NH₂;
and pharmaceutically compatible salts thereof;
excluding the compound where
R1=H, R2=F, R3=methyl and Cyc1-R4=4—OCH₃-phenyl.

All references hereinafter to “compound(s) of the formula I” refer to compound(s) of the formula I as described above, and to the salts, solvates and physiologically functional derivatives thereof, as described herein.

When radicals or substituents can occur more than once in the compounds of the formula I, they may each independently have the definitions specified and be the same or different.

The symbols in the formula I are preferably each independently defined as follows:

Preference is given to compounds of the formula I in which

Ra, Rb, Rc are each hydrogen.

Preference is also given to compounds of the formula I in which

Ra is —COO—(C₁-C₆)-alkyl; and
Rb, Rc are each hydrogen.

Preference is given to compounds of the formula I in which

R1 and R2 are each F.

Preference is given to compounds of the formula I in which:
R3 is hydrogen, F, Cl, Br, CF₃, OCF₃, methyl, methoxy, cyclopropyl, CH₂-cyclopropyl.

Particular preference is given to compounds of the formula I in which:

R3 is F, Cl, Br, CF₃, OCF₃, methyl, methoxy.

Particular preference is given to compounds of the formula I in which:
one R4, R5, R6 or R7 is F, Cl, CF₃, OH, COOH, (C₁-C₆)-alkyl, (C₂-C₆)-alkenyl, O—(C₁-C₆)-alkyl, HO—(C₁-C₆)-alkylene or (C₁-C₆)-alkylene-O—(C₁-C₆)-alkyl, where one, more than one or all hydrogen(s) in the alkyl and O-alkyl radicals may be replaced by fluorine;
the others are each hydrogen.

Particular preference is given to compounds of the formula I in which
one R4, R5, R6 or R7 is F, Cl, CF₃, OH, (C₁-C₆)-alkyl, (C₂-C₆)-alkenyl, O—(C₁-C₆)-alkyl, HO—(C₁-C₆)-alkylene or (C₁-C₆)-alkylene-O—(C₁-C₆)-alkyl;
the others are each hydrogen.

Very particular preference is given to compounds of the formula I in which

R4 is C₁, CF₃, OCF₃, ethyl, methoxy, ethoxy;
R5, R6, R7 are each hydrogen.

A preferred embodiment is that of compounds of the formula I in which

### Cyc1 is

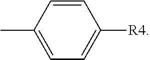

A further preferred embodiment is that of compounds of the formula I in which

### Cyc1 is

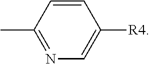

A further preferred embodiment is that of compounds of the formula I in which

### Cyc1 is

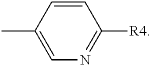

A further preferred embodiment is that of compounds of the formula I in which

### Cyc1 is

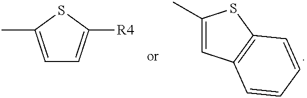

The alkyl radicals in the R3, R4, R5, R6 and R7 substitutes may be either straight-chain or branched. Halogen is understood to mean F, Cl, Br, I, preferably F and Cl.

The invention relates to compounds of the formula I in the form of their tautomers, racemates, racemic mixtures and pure enantiomers, and to their diastereomers and mixtures thereof. The present invention encompasses all these isomeric and, if appropriate, tautomeric forms of the compounds of the formula I. These isomeric forms may be obtained by known methods, even if not expressly described (in some cases).

Pharmaceutically acceptable salts are, because their solubility in water is greater than that of the initial or basic compounds, particularly suitable for medical applications. These salts must have a pharmaceutically acceptable anion or cation. Suitable pharmaceutically acceptable acid addition salts of the compounds of the invention are salts of inorganic acids such as hydrochloric acid, hydrobromic acid, phosphoric acid, metaphosphoric acid, nitric acid and sulfuric acid, and of organic acids, for example acetic acid, benzenesulfonic acid, benzoic acid, citric acid, ethanesulfonic acid, fumaric acid, gluconic acid, glycolic acid, isethionic acid, lactic acid, lactobionic acid, maleic acid, malic acid, methanesulfonic acid, succinic acid, p-toluenesulfonic acid and tartaric acid. Suitable pharmaceutically acceptable basic salts are ammonium salts, alkali metal salts (such as sodium and potassium salts), alkaline earth metal salts (such as magnesium and calcium salts) and salts of trometamol (2-amino-2-hydroxymethyl-1,3-propanediol), diethanolamine, lysine or ethylenediamine.

Salts with a pharmaceutically unacceptable anion, for example trifluoroacetate, likewise belong within the framework of the invention as useful intermediates for the preparation or purification of pharmaceutically acceptable salts and/or for use in nontherapeutic, for example in vitro, applications.

The term “physiologically functional derivative” used herein refers to any physiologically tolerated derivative of a compound of the formula I of the invention, for example an ester, which on administration to a mammal, for example a human, is able to form (directly or indirectly) a compound of the formula I or an active metabolite thereof.

Physiologically functional derivatives also include prodrugs of the compounds of the invention, as described, for example, in H. Okada et al., Chem. Pharm. Bull. 1994, 42, 57-61. Such prodrugs can be metabolized in vivo to a compound of the invention. These prodrugs may themselves be active or not.

The compounds of the invention may also exist in various polymorphous forms, for example as amorphous and crystalline polymorphous forms. All polymorphous forms of the compounds of the invention belong within the framework of the invention and are a further aspect of the invention.

All references to “compound(s) of formula I” hereinafter refer to compound(s) of the formula I as described above, and their salts, solvates and physiologically functional derivatives as described herein.

### Use

The invention further relates to the use of compounds of the formula I and pharmaceutical compositions thereof for inhibition of SGLT2 (sodium dependent glucose transporter 2).

SGLT2 is responsible for the reabsorption of D-glucose from the glomerular filtrate of the kidneys (E. M. Wright et al., Am. J. Physiol. 2001, 263: F459-F465).

Inhibition of tubular reabsorption of glucose contributes to lowering the blood glucose concentration. Inhibitors of SGLT2 are therefore suitable for treatment, control and prophylaxis of metabolic disorders, especially of diabetes mellitus.

The inventive compounds are also notable for a particularly high selectivity for SGLT2 compared to the SGLT1 receptor. This selectivity is enhanced further in the case of the difluoro compounds.

Inventive compounds esterified on the glucose unit act as prodrugs. In in vitro test methods, they exhibit poor IC50 values for SGLT2. They are nevertheless selective SGLT2 inhibitors, as demonstrated by the glucose excretion data of the in vivo tests.

The compounds of the formula I are notable for favorable effects on the glucose metabolism; more particularly, they lower the blood sugar level and are suitable for treatment of type 1 and type 2 diabetes. The compounds can therefore be used alone or in combination with further active ingredients which lower blood sugar (antidiabetics).

The compounds of the formula I are additionally suitable for prevention and treatment of diabetic late damage, for example nephropathy, retinopathy, neuropathy, and also syndrome X, obesity, myocardinal infarction, peripheral arterial occlusive diseases, thromboses, arteriosclerosis, inflammation, immune disease, autoimmune disease, for example AIDS, asthma, osteoporosis, cancer, psoriasis, Alzheimer's, schizophrenia and infectious disease; preference is given to the treatment of type 1 and type 2 diabetes and for the prevention and treatment of diabetic late damage, syndrome X and obesity.

### Formulations

The amount of a compound of formula I necessary to achieve the desired biological effect depends on a number of factors, for example the specific compound chosen, the intended use, the mode of administration and the clinical condition of the patient. The daily dose is generally in the range from 0.3 mg to 100 mg (typically from 3 mg to 50 mg) per day and per kilogram of body weight, for example 3-10 mg/kg/day. Single-dose formulations which can be administered orally, for example tablets or capsules, may contain, for example, from 1.0 to 1000 mg, typically from 10 to 600 mg. For the therapy of the abovementioned conditions, the compounds of formula I may be used as the compound itself, but they are preferably in the form of a pharmaceutical composition with an acceptable carrier. The carrier must, of course, be acceptable in the sense that it is compatible with the other ingredients of the composition and is not harmful for the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including other compounds of formula I. The pharmaceutical compositions of the invention can be produced by one of the known pharmaceutical methods, which essentially consist of mixing the ingredients with pharmacologically acceptable carriers and/or excipients.

Pharmaceutical compositions of the invention are those suitable for oral, rectal peroral (for example sublingual) and administration, although the most suitable mode of administration depends in each individual case on the nature and severity of the condition to be treated and on the nature of the compound of formula I used in each case. Coated formulations and coated slow-release formulations also belong within the framework of the invention. Preference is given to acid- and gastric juice-resistant formulations. Suitable coatings resistant to gastric juice comprise cellulose acetate phthalate, polyvinyl acetate phthalate, hydroxypropylmethylcellulose phthalate and anionic polymers of methacrylic acid and methyl methacrylate.

Suitable pharmaceutical preparations for oral administration may be in the form of separate units such as, for example, capsules, cachets, suckable tablets or tablets, each of which contains a defined amount of the compound of formula I; as powders or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is molded if necessary. Thus, for example, a tablet can be produced by compressing or molding a powder or granules of the compound, where appropriate with one or more additional ingredients. Compressed tablets can be produced by tableting the compound in free-flowing form such as, for example, a powder or granules, where appropriate mixed with a binder, glidant, inert diluent and/or one (or more) surface-active/dispersing agent(s) in a suitable machine. Molded tablets can be produced by molding the compound, which is in powder form and is moistened with an inert liquid diluent, in a suitable machine.

Pharmaceutical compositions which are suitable for peroral (sublingual) administration comprise suckable tablets which contain a compound of formula I with a flavoring, normally sucrose and gum arabic or tragacanth, and pastilles which comprise the compound in an inert base such as gelatin and glycerol or sucrose and gum arabic.

Pharmaceutical compositions suitable for rectal administration are preferably in the form of single-dose suppositories. These can be produced by mixing a compound of the formula I with one or more conventional solid carriers, for example cocoa butter, and shaping the resulting mixture.

Combinations with Other Medicaments

The compounds of the invention can be administered alone or in combination with one or more further pharmacologically active substances which have, for example, beneficial effects on metabolic disturbances or disorders frequently associated therewith. They can be combined with the compounds of the invention of the formula I in particular for a synergistic improvement in action. The active ingredient combination can be administered either by separate administration of the active ingredients to the patient or in the form of combination products in which a plurality of active ingredients are present in one pharmaceutical preparation. When the active ingredients are administered by separate administration of the active ingredients, this can be done simultaneously or successively.

Further suitable active ingredients for the combination preparations are:

all antidiabetics which are mentioned in the Rote Liste 2007, chapter 12; all weight-reducing agents/appetite suppressants which are mentioned in the Rote Liste 2007, chapter 1; all diuretics which are mentioned in the Rote Liste 2007, chapter 36; all lipid-lowering agents which are mentioned in the Rote Liste 2007, chapter 58. Most of the active ingredients mentioned hereinafter are disclosed in the USP Dictionary of USAN and International Drug Names, US Pharmacopeia, Rockville 2006.

Antidiabetics include insulin and insulin derivatives, for example Lantus® (see www.lantus.com) or HMR 1964 or Levemir® (insulin detemir), Humalog® (Insulin Lispro), Humulin®, VIAject™, SuliXen® or those as described in WO2005005477 (Novo Nordisk), fast-acting insulins (see U.S. Pat. No. 6,221,633), inhalable insulins, for example Exubera®, Nasulin™, or oral insulins, for example IN-105 (Nobex) or Orallyn™ (Generex Biotechnology), or Technosphere® Insulin (MannKind) or Cobalamin™ oral insulin, or insulins as described in WO2007128815, WO2007128817, WO2008034881, WO2008049711, or insulins which can be administered transdermally;

GLP-1 derivatives and GLP-1 agonists, for example exenatide or specific formulations thereof, as described, for example, in WO2008061355, liraglutide, taspoglutide (R-1583), albiglutide, lixisenatide or those which have been disclosed in WO 98/08871, WO2005027978, WO2006037811, WO2006037810 by Novo Nordisk A/S, in WO 01/04156 by Zealand or in WO 00/34331 by Beaufour-Ipsen, pramlintide acetate (Symlin; Amylin Pharmaceuticals), AVE-0010, BIM-51077 (R-1583, ITM-077), PC-DAC:Exendin-4 (an exendin-4 analog which is bonded covalently to recombinant human albumin), CVX-73, CVX-98 and CVx-96 (GLP-1 analog which is bonded covalently to a monoclonal antibody which has specific binding sites for the GLP-1 peptide), CNTO-736 (a GLP-1 analog which is bonded to a domain which includes the Fc portion of an antibody), PGC-GLP-1 (GLP-1 bonded to a nanocarrier), agonists, as described, for example, in D. Chen et al., Proc. Natl. Acad. Sci. USA 104 (2007) 943, those as described in WO2006124529, WO2007124461, WO2008062457, WO2008082274, WO2008101017, WO2008081418, WO2008112939, WO2008112941, WO2008113601, WO2008116294, WO2008116648, WO2008119238, peptides, for example obinepitide (TM-30338), amylin receptor agonists, as described, for example, in WO2007104789, analogs of the human GLP-1, as described in WO2007120899, WO2008022015, WO2008056726, and orally active hypoglycemic ingredients.

Antidiabetics also include agonists of the glucose-dependent insulinotropic polypeptide (GIP) receptor, as described, for example, in WO2006121860.

Antidiabetics also include the glucose-dependent insulinotropic polypeptide (GIP), and also analogous compounds, as described, for example, in WO2008021560.

Antidiabetics also include analogs and derivatives of fibroblast growth factor 21 (FGF-21).

The orally active hypoglycemic ingredients preferably include

sulfonylureas,
biguanidines,
meglitinides,
oxadiazolidinediones,
thiazolidinediones,
PPAR and RXR modulators,
glucosidase inhibitors,
inhibitors of glycogen phosphorylase,
glucagon receptor antagonists,
glucokinase activators,
inhibitors of fructose 1,6-bisphosphatase
modulators of glucose transporter 4 (GLUT4),
inhibitors of glutamine-fructose-6-phosphate amidotransferase (GFAT), GLP-1 agonists,
potassium channel openers, for example pinacidil, cromakalim, diazoxide, or those as described in R. D. Carr et al., Diabetes 52, 2003, 2513.2518, in J. B. Hansen et al, Current Medicinal Chemistry 11, 2004, 1595-1615, in T. M. Tagmose et al., J. Med. Chem. 47, 2004, 3202-3211 or in M. J. Coghlan et al., J. Med. Chem. 44, 2001, 1627-1653, or those which have been disclosed in WO 97/26265 and WO 99/03861 by Novo Nordisk A/S,
active ingredients which act on the ATP-dependent potassium channel of the beta cells,
inhibitors of dipeptidylpeptidase IV (DPP-IV),
insulin sensitizers,
inhibitors of liver enzymes involved in stimulating gluconeogenesis and/or glycogenolysis,
modulators of glucose uptake, of glucose transport and of glucose reabsorption,
modulators of sodium-dependent glucose transporter 1 or 2 (SGLT1, SGLT2),
inhibitors of 11-beta-hydroxysteroid dehydrogenase-1 (11β-HSD1),
inhibitors of protein tyrosine phosphatase 1B (PTP-1B),
nicotinic acid receptor agonists,
inhibitors of hormone-sensitive or endothelial lipases,
inhibitors of acetyl-CoA carboxylase (ACC1 and/or ACC2) or
inhibitors of GSK-3 beta.

Also included are compounds which modify the metabolism, such as active antihyperlipidemic ingredients and active antilipidemic ingredients,

HMGCoA reductase inhibitors,
farnesoid X receptor (FXR) modulators,
fibrates,
cholesterol reabsorption inhibitors,
CETP inhibitors,
bile acid reabsorption inhibitors,
MTP inhibitors,
agonists of estrogen receptor gamma (ERRγ agonists),
sigma-1 receptor antagonists,
antagonists of the somatostatin 5 receptor (SST5 receptor);
compounds which reduce food intake, and
compounds which increase thermogenesis.

In one embodiment of the invention, the compound of the formula I is administered in combination with insulin.

In one embodiment, the compound of the formula I is administered in combination with an active ingredient which acts on the ATP-dependent potassium channel of the beta cells, for example sulfonylureas, for example tolbutamide, glibenclamide, glipizide, gliclazide or glimepiride.

In one embodiment, the compound of the formula I is administered in combination with a tablet which comprises both glimepiride, which is released rapidly, and metformin, which is released over a longer period (as described, for example, in US2007264331, WO2008050987, WO2008062273).

In one embodiment, the compound of the formula I is administered in combination with a biguanide, for example metformin.

In another embodiment, the compound of the formula I is administered in combination with a meglitinide, for example repaglinide, nateglinide or mitiglinide.

In a further embodiment, the compound of the formula I is administered with a combination of mitiglinide with a glitazone, e.g. pioglitazone hydrochloride.

In a further embodiment, the compound of the formula I is administered with a combination of mitiglinide with an alpha-glucosidase inhibitor.

In a further embodiment, the compound of the formula I is administered in combination with antidiabetic compounds, as described in WO2007095462, WO2007101060, WO2007105650.

In a further embodiment, the compound of the formula I is administered in combination with antihypoglycemic compounds, as described in WO2007137008, WO2008020607.

In one embodiment, the compound of the formula I is administered in combination with a thiazolidinedione, for example troglitazone, ciglitazone, pioglitazone, rosiglitazone or the compounds disclosed in WO 97/41097 by Dr. Reddy's Research Foundation, especially 5-[[4-[(3,4-dihydro-3-methyl-4-oxo-2-quinazolinylmethoxy]-phenyl]methyl]-2,4-thiazolidinedione.

In one embodiment of the invention, the compound of the formula I is administered in combination with a PPAR gamma agonist, for example rosiglitazone, pioglitazone, JTT-501, GI 262570, R-483, CS-011 (rivoglitazone), DRL-17564, DRF-2593 (balaglitazone), INT-131, T-2384, or those as described in WO2005086904, WO2007060992, WO2007100027, WO2007103252, WO2007122970, WO2007138485, WO2008006319, WO2008006969, WO2008010238, WO2008017398, WO2008028188, WO2008066356, WO2008084303, WO2008089461-WO2008089464, WO2008093639, WO2008096769, WO2008096820, WO2008096829, US2008194617, WO2008099944, WO2008108602, WO2008109334, WO2008126731, WO2008126732.

In one embodiment of the invention, the compound of the formula I is administered in combination with Competact™, a solid combination of pioglitazone hydrochloride with metformin hydrochloride.

In one embodiment of the invention, the compound of the formula I is administered in combination with Tandemact™, a solid combination of pioglitazone with glimepiride.

In a further embodiment of the invention, the compound of the formula I is administered in combination with a solid combination of pioglitazone hydrochloride with an angiotensin II agonist, for example TAK-536.

In one embodiment of the invention, the compound of the formula I is administered in combination with a PPAR alpha agonist or mixed PPAR alpha/PPAR delta agonist, for example GW9578, GW-590735, K-111, LY-674, KRP-101, DRF-10945, LY-518674, CP-900691, BMS-687453, BMS-711939, or those as described in WO2001040207, WO2002096894, WO2005097076, WO2007056771, WO2007087448, WO2007089667, WO2007089557, WO2007102515, WO2007103252, JP2007246474, WO2007118963, WO2007118964, WO2007126043, WO2008006043, WO2008006044, WO2008012470, WO2008035359, WO2008087365, WO2008087366, WO2008087367, WO2008117982.

In one embodiment of the invention, the compound of the formula I is administered in combination with a mixed PPAR alpha/gamma agonist, for example naveglitazar, LY-510929, ONO-5129, E-3030, AVE 8042, AVE 8134, AVE 0847, CKD-501 (lobeglitazone sulfate), MBX-213, KY-201 or as described in WO 00/64888, WO 00/64876, WO03/020269, WO2004024726, WO2007099553, US2007276041, WO2007085135, WO2007085136, WO2007141423, WO2008016175, WO2008053331, WO2008109697, WO2008109700, WO2008108735 or in J. P. Berger et al., TRENDS in Pharmacological Sciences 28(5), 244-251, 2005.

In one embodiment of the invention, the compound of the formula I is administered in combination with a PPAR delta agonist, for example GW-501516, or as described in WO2006059744, WO2006084176, WO2006029699, WO2007039172, WO2007039178, WO2007071766, WO2007101864, US2007244094, WO2007119887, WO2007141423, US2008004281, WO2008016175, WO2008066356, WO2008071311, WO2008084962, US2008176861.

In one embodiment of the invention, the compound of the formula I is administered in combination with a pan-SPPARM (selective PPAR modulator alpha, gamma, delta), for example GFT-505, or those as described in WO2008035359.

In one embodiment, the compound of the formula I is administered in combination with metaglidasen or with MBX-2044 or other partial PPAR gamma agonists/antagonists.

In one embodiment, the compound of the formula I is administered in combination with an α-glucosidase inhibitor, for example miglitol or acarbose, or those as described, for example, in WO2007114532, WO2007140230, US2007287674, US2008103201, WO2008065796, WO2008082017.

In one embodiment, the compound of the formula I is administered in combination with an inhibitor of glycogen phosphorylase, for example PSN-357 or FR-258900, or those as described in WO2003084922, WO2004007455, WO2005073229-31, WO2005067932, WO2008062739, WO2008099000, WO2008113760.

In one embodiment, the compound of the formula I is administered in combination with glucagon receptor antagonists, for example A-770077 or NNC-25-2504 or as described in WO2004100875, WO2005065680, WO2006086488, WO2007047177, WO2007106181, WO2007111864, WO2007120270, WO2007120284, WO2007123581, WO2007136577, WO2008042223, WO2008098244.

In a further embodiment, the compound of the formula I is administered in combination with an antisense compound, e.g. ISIS-325568, which inhibits the production of the glucagon receptor.

In one embodiment, the compound of the formula I is administered in combination with activators of glucokinase, for example LY-2121260 (WO2004063179), PSN-105, PSN-110, GKA-50, or those as described, for example, in WO2004072031, WO2004072066, WO2005080360, WO2005044801, WO2006016194, WO2006058923, WO2006112549, WO2006125972, WO2007017549, WO2007017649, WO2007007910, WO2007007040-42, WO2007006760-61, WO2007006814, WO2007007886, WO2007028135, WO2007031739, WO2007041365, WO2007041366, WO2007037534, WO2007043638, WO2007053345, WO2007051846, WO2007051845, WO2007053765, WO2007051847, WO2007061923, WO2007075847, WO2007089512, WO2007104034, WO2007117381, WO2007122482, WO2007125103, WO2007125105, US2007281942, WO2008005914, WO2008005964, WO2008043701, WO2008044777, WO2008047821, US2008096877, WO2008050117, WO2008050101, WO2008059625, US2008146625, WO2008078674, WO2008079787, WO2008084043, WO2008084044, WO2008084872, WO2008089892, WO2008091770, WO2008075073, WO2008084043, WO2008084044, WO2008084872, WO2008084873, WO2008089892, WO2008091770, JP2008189659, WO2008104994, WO2008111473, WO2008116107, WO2008118718, WO2008120754.

In one embodiment, the compound of the formula I is administered in combination with an inhibitor of gluconeogenesis, as described, for example, in FR-225654, WO2008053446.

In one embodiment, the compound of the formula I is administered in combination with inhibitors of fructose 1,6-bisphosphatase (FBPase), for example MB-07729, CS-917 (MB-06322) or MB-07803, or those as described in WO2006023515, WO2006104030, WO2007014619, WO2007137962, WO2008019309, WO2008037628.

In one embodiment, the compound of the formula I is administered in combination with modulators of glucose transporters 4 (GLUT4), for example KST-48 (D.-O. Lee et al.: Arzneim.-Forsch. Drug Res. 54 (12), 835 (2004)).

In one embodiment, the compound of the formula I is administered in combination with inhibitors of glutamine:fructose-6-phosphate amidotransferase (GFAT), as described, for example, in WO2004101528.

In one embodiment, the compound of the formula I is administered in combination with inhibitors of dipeptidyl peptidase IV (DPP-IV), for example vildagliptin (LAF-237), sitagliptin (MK-0431), sitagliptin phosphate, saxagliptin ((BMS-477118), GSK-823093, PSN-9301, SYR-322, SYR-619, TA-6666, TS-021, GRC-8200 (melogliptin), GW-825964X, KRP-104, DP-893, ABT-341, ABT-279 or another salt thereof, S-40010, S-40755, PF-00734200, BI-1356, PHX-1149, alogliptin benzoate, linagliptin, melogliptin or those compounds as described in WO2003074500, WO2003106456, WO2004037169, WO200450658, WO2005037828, WO2005058901, WO2005012312, WO2005/012308, WO2006039325, WO2006058064, WO2006015691, WO2006015701, WO2006015699, WO2006015700, WO2006018117, WO2006099943, WO2006099941, JP2006160733, WO2006071752, WO2006065826, WO2006078676, WO2006073167, WO2006068163, WO2006085685, WO2006090915, WO2006104356, WO2006127530, WO2006111261, US2006890898, US2006803357, US2006303661, WO2007015767 (LY-2463665), WO2007024993, WO2007029086, WO2007063928, WO2007070434, WO2007071738, WO2007071576, WO2007077508, WO2007087231, WO2007097931, WO2007099385, WO2007100374, WO2007112347, WO2007112669, WO2007113226, WO2007113634, WO2007115821, WO2007116092, US2007259900, EP1852108, US2007270492, WO2007126745, WO2007136603, WO2007142253, WO2007148185, WO2008017670, US2008051452, WO2008027273, WO2008028662, WO2008029217, JP2008031064, JP2008063256, WO2008033851, WO2008040974, WO2008040995, WO2008060488, WO2008064107, WO2008066070, WO2008077597, JP2008156318, WO2008087560, WO2008089636, WO2008093960, WO2008096841, WO2008101953, WO2008118848, WO2008119005, WO2008119208, WO2008120813, WO2008121506.

In one embodiment, the compound of the formula I is administered in combination with Janumet™, a solid combination of sitagliptin phosphate with metformin hydrochloride.

In one embodiment, the compound of the formula I is administered in combination with Eucreas®, a solid combination of vildagliptin with metformin hydrochloride.

In a further embodiment, the compound of the formula I is administered in combination with a solid combination of alogliptin benzoate with pioglitazone.

In one embodiment, the compound of the formula I is administered in combination with a solid combination of a salt of sitagliptin with metformin hydrochloride.

In one embodiment, the compound of the formula I is administered in combination with a combination of a DPP-IV inhibitor with omega-3 fatty acids or omega-3 fatty acid esters, as described, for example, in WO2007128801.

In one embodiment, the compound of the formula I is administered in combination with a solid combination of a salt of sitagliptin with metformin hydrochloride.

In one embodiment, the compound of the formula I is administered in combination with a substance which enhances insulin secretion, for example KCP-265 (WO2003097064), or those as described in WO2007026761, WO2008045484, US2008194617.

In one embodiment, the compound of the formula I is administered in combination with agonists of the glucose-dependent insulinotropic receptor (GDIR), for example APD-668.

In one embodiment of the invention, the compound of the formula I is administered in combination with an ATP citrate lyase inhibitor, for example SB-204990.

In one embodiment, the compound of the formula I is administered in combination with modulators of the sodium-dependent glucose transporter 1 or 2 (SGLT1, SGLT2), for example KGA-2727, T-1095, SGL-0010, AVE 2268, SAR 7226, SGL-5083, SGL-5085, SGL-5094, ISIS-388626, sergliflozin or dapagliflozin, or as described, for example, in WO2004007517, WO200452903, WO200452902, PCT/EP2005/005959, WO2005085237, JP2004359630, WO2005121161, WO2006018150, WO2006035796, WO2006062224, WO2006058597, WO2006073197, WO2006080577, WO2006087997, WO2006108842, WO2007000445, WO2007014895, WO2007080170, WO2007093610, WO2007126117, WO2007128480, WO2007129668, US2007275907, WO2007136116, WO2007143316, WO2007147478, WO2008001864, WO2008002824, WO2008013277, WO2008013280, WO2008013321, WO2008013322, WO2008016132, WO2008020011, JP2008031161, WO2008034859, WO2008042688, WO2008044762, WO2008046497, WO2008049923, WO2008055870, WO2008055940, WO2008069327, WO2008070609, WO2008071288, WO2008072726, WO2008083200, WO2008090209, WO2008090210, WO2008101586, WO2008101939, WO2008116179, WO2008116195, US2008242596 or by A. L. Handlon in Expert Opin. Ther. Patents (2005) 15(11), 1531-1540.

In one embodiment, the compound of the formula I is administered in combination with inhibitors of 11-beta-hydroxysteroid dehydrogenase 1 (11β-HSD1), for example BVT-2733, JNJ-25918646, INCB-13739, INCB-20817, D10-92 ((−)-ketoconazole) or those as described, for example, in WO200190090-94, WO200343999, WO2004112782, WO200344000, WO200344009, WO2004112779, WO2004113310, WO2004103980, WO2004112784, WO2003065983, WO2003104207, WO2003104208, WO2004106294, WO2004011410, WO2004033427, WO2004041264, WO2004037251, WO2004056744, WO2004058730, WO2004065351, WO2004089367, WO2004089380, WO2004089470-71, WO2004089896, WO2005016877, WO2005063247, WO2005097759, WO2006010546, WO2006012227, WO2006012173, WO2006017542, WO2006034804, WO2006040329, WO2006051662, WO2006048750, WO2006049952, WO2006048331, WO2006050908, WO2006024627, WO2006040329, WO2006066109, WO2006074244, WO2006078006, WO2006106423, WO2006132436, WO2006134481, WO2006134467, WO2006135795, WO2006136502, WO2006138508, WO2006138695, WO2006133926, WO2007003521, WO2007007688, US2007066584, WO2007029021, WO2007047625, WO2007051811, WO2007051810, WO2007057768, WO2007058346, WO2007061661, WO2007068330, WO2007070506, WO2007087150, WO2007092435, WO2007089683, WO2007101270, WO2007105753, WO2007107470, WO2007107550, WO2007111921, US2007207985, US2007208001, WO2007115935, WO2007118185, WO2007122411, WO2007124329, WO2007124337, WO2007124254, WO2007127688, WO2007127693, WO2007127704, WO2007127726, WO2007127763, WO2007127765, WO2007127901, US2007270424, JP2007291075, WO2007130898, WO2007135427, WO2007139992, WO2007144394, WO2007145834, WO2007145835, WO2007146761, WO2008000950, WO2008000951, WO2008003611, WO2008005910, WO2008006702, WO2008006703, WO2008011453, WO2008012532, WO2008024497, WO2008024892, WO2008032164, WO2008034032, WO2008043544, WO2008044656, WO2008046758, WO2008052638, WO2008053194, WO2008071169, WO2008074384, WO2008076336, WO2008076862, WO2008078725, WO2008087654, WO2008088540, WO2008099145, WO2008101885, WO2008101886, WO2008101907, WO2008101914, WO2008106128, WO2008110196, WO2008119017, WO2008120655, WO2008127924.

In one embodiment, the compound of the formula I is administered in combination with inhibitors of protein tyrosine phosphatase 1B (PTP-1B), as described, for example, in WO200119830-31, WO200117516, WO2004506446, WO2005012295, WO2005116003, WO2005116003, WO2006007959, DE 10 2004 060542.4, WO2007009911, WO2007028145, WO2007067612-615, WO2007081755, WO2007115058, US2008004325, WO2008033455, WO2008033931, WO2008033932, WO2008033934, WO2008089581.

In one embodiment of the invention, the compound of the formula I is administered in combination with an agonist of GPR109A (HM74A receptor agonists; NAR agonists (nicotinic acid receptor agonists)), for example nicotinic acid or “extended release niacin” in conjunction with MK-0524A (laropiprant) or MK-0524, or those compounds as described in WO2004041274, WO2006045565, WO2006045564, WO2006069242, WO2006085108, WO2006085112, WO2006085113, WO2006124490, WO2006113150, WO2007017261, WO2007017262, WO2007017265, WO2007015744, WO2007027532, WO2007092364, WO2007120575, WO2007134986, WO2007150025, WO2007150026, WO2008016968, WO2008051403, WO2008086949, WO2008091338, WO2008097535, WO2008099448, US2008234277, WO2008127591.

In another embodiment of the invention, the compound of the formula I is administered in combination with a solid combination of niacin with simvastatin.

In another embodiment of the invention, the compound of the formula I is administered in combination with nicotinic acid or “extended release niacin” in conjunction with MK-0524A (laropiprant).

In a further embodiment of the invention, the compound of the formula I is administered in combination with nicotinic acid or “extended release niacin” in conjunction with MK-0524A (laropiprant) and with simvastatin.

In one embodiment of the invention, the compound of the formula I is administered in combination with nicotinic acid or another nicotinic acid receptor agonist and a prostaglandin DP receptor antagonist, for example those as described in WO2008039882.

In another embodiment of the invention, the compound of the formula I is administered in combination with an agonist of GPR116, as described, for example, in WO2006067531, WO2006067532.

In one embodiment, the compound of the formula I is administered in combination with modulators of GPR40, as described, for example, in WO2007013689, WO2007033002, WO2007106469, US2007265332, WO2007123225, WO2007131619, WO2007131620, WO2007131621, US2007265332, WO2007131622, WO2007136572, WO2008001931, WO2008030520, WO2008030618, WO2008054674, WO2008054675, WO2008066097, US2008176912.

In one embodiment, the compound of the formula I is administered in combination with modulators of GPR119 (G-protein-coupled glucose-dependent insulinotropic receptor), for example PSN-119-1, PSN-821, PSN-119-2, MBX-2982, or those as described, for example, in WO2004065380, WO2005061489 (PSN-632408), WO2006083491, WO2007003960-62 and WO2007003964, WO2007035355, WO2007116229, WO2007116230, WO2008005569, WO2008005576, WO2008008887, WO2008008895, WO2008025798, WO2008025799, WO2008025800, WO2008070692, WO2008076243, WO200807692, WO2008081204, WO2008081205, WO2008081206, WO2008081207, WO2008081208, WO2008083238, WO2008085316, WO2008109702.

In a further embodiment, the compound of the formula I is administered in combination with modulators of GPR120, as described, for example, in EP1688138, WO2008066131, WO2008066131, WO2008103500, WO2008103501.

In one embodiment, the compound of the formula I is administered in combination with inhibitors of hormone-sensitive lipase (HSL) and/or phospholipases, as described, for example, in WO2005073199, WO2006074957, WO2006087309, WO2006111321, WO2007042178, WO2007119837, WO2008122352, WO2008122357.

In one embodiment, the compound of the formula I is administered in combination with inhibitors of endothelial lipase, as described, for example, in WO2006111321, WO2006131233, WO2006131232, WO2006131231, WO2007042178, WO2007045392, WO2007045393, WO2007110216, WO2007110215, WO2008122357, WO2008122352.

In one embodiment, the compound of the formula I is administered in combination with a phospholipase A2 inhibitor, for example darapladib or A-002, or those as described in WO2008048866, WO20080488867.

In one embodiment, the compound of the formula I is administered in combination with myricitrin, a lipase inhibitor (WO2007119827).

In one embodiment, the compound of the formula I is administered in combination with an inhibitor of glycogen synthase kinase-3 beta (GSK-3 beta), as described, for example, in US2005222220, WO2005085230, WO2005111018, WO2003078403, WO2004022544, WO2003106410, WO2005058908, US2005038023, WO2005009997, US2005026984, WO2005000836, WO2004106343, EP1460075, WO2004014910, WO2003076442, WO2005087727, WO2004046117, WO2007073117, WO2007083978, WO2007120102, WO2007122634, WO2007125109, WO2007125110, US2007281949, WO2008002244, WO2008002245, WO2008016123, WO2008023239, WO2008044700, WO2008056266, WO2008057940, WO2008077138, EP1939191, EP1939192, WO2008078196, WO2008094992, WO2008112642, WO2008112651, WO2008113469, WO2008121063, WO2008121064.

In one embodiment, the compound of the formula I is administered in combination with an inhibitor of phosphoenolpyruvate carboxykinase (PEPCK), for example those as described in WO2004074288.

In one embodiment, the compound of the formula I is administered in combination with an inhibitor of phosphoinositide kinase-3 (PI3K), for example those as described in WO2008027584, WO2008070150, WO2008125833, WO2008125835, WO2008125839.

In one embodiment, the compound of the formula I is administered in combination with an inhibitor of serum/glucocorticoid-regulated kinase (SGK), as described, for example, in WO2006072354, WO2007093264, WO2008009335, WO2008086854.

In one embodiment, the compound of the formula I is administered in combination with a modulator of the glucocorticoid receptor, as described, for example, in WO2008057855, WO2008057856, WO2008057857, WO2008057859, WO2008057862, WO2008059867, WO2008059866, WO2008059865, WO2008070507, WO2008124665, WO2008124745.

In one embodiment, the compound of the formula I is administered in combination with a modulator of the mineralocorticoid receptor (MR), for example drospirenone, or those as described in WO2008104306, WO2008119918.

In one embodiment, the compound of the formula I is administered in combination with an inhibitor of protein kinase C beta (PKC beta), for example ruboxistaurin, or those as described in WO2008096260, WO2008125945.

In one embodiment, the compound of the formula I is administered in combination with an inhibitor of protein kinase D, for example doxazosin (WO2008088006).

In a further embodiment, the compound of the formula I is administered in combination with an activator of the AMP-activated protein kinase (AMPK), as described, for example, in WO2007062568, WO2008006432, WO2008016278, WO2008016730, WO2008083124.

In one embodiment, the compound of the formula I is administered in combination with an inhibitor of ceramide kinase, as described, for example, in WO2007112914, WO2007149865.

In a further embodiment, the compound of the formula I is administered in combination with an inhibitor of MAPK-interacting kinase 1 or 2 (MNK1 or 2), as described, for example, in WO2007104053, WO2007115822, WO2008008547, WO2008075741.

In one embodiment, the compound of the formula I is administered in combination with inhibitors of “I-kappaB kinase” (IKK inhibitors), as described, for example, in WO2001000610, WO2001030774, WO2004022057, WO2004022553, WO2005097129, WO2005113544, US2007244140, WO2008099072, WO2008099073, WO2008099073, WO2008099074, WO2008099075.

In another embodiment, the compound of the formula I is administered in combination with inhibitors of NF-kappaB (NFKB) activation, for example salsalate.

In a further embodiment, the compound of the formula I is administered in combination with inhibitors of ASK-1 (apoptosis signal-regulating kinase 1), as described, for example, in WO2008016131.

In one embodiment of the invention, the compound of the formula I is administered in combination with an HMG-CoA reductase inhibitor such as simvastatin, fluvastatin, pravastatin, lovastatin, atorvastatin, cerivastatin, rosuvastatin, pitavastatin, L-659699, BMS-644950, or those as described in US2007249583, WO2008083551.

In a further embodiment of the invention, the compound of the formula I is administered in combination with a farnesoid X receptor (FXR) modulator, for example WAY-362450 or those as described in WO2003099821, WO2005056554, WO2007052843, WO2007070796, WO2007092751, JP2007230909, WO2007095174, WO2007140174, WO2007140183, WO2008000643, WO2008002573, WO2008025539, WO2008025540, JP2008214222.

In another embodiment of the invention, the compound of the formula I is administered in combination with a ligand of the liver X receptor (LXR), as described, for example, in WO2007092965, WO2008041003, WO2008049047, WO2008065754, WO2008073825, US2008242677.

In one embodiment of the invention, the compound of the formula I is administered in combination with a fibrate, for example fenofibrate, clofibrate, bezafibrate, or those as are described in WO2008093655.

In one embodiment of the invention, the compound of the formula I is administered in combination with fibrates, for example the choline salt of fenofibrate (SLV-348).

In one embodiment of the invention, the compound of the formula I is administered in combination with fibrates, for example the choline salt of fenofibrate and an HMG-CoA reductase inhibitor, for example rosuvastatin.

In a further embodiment of the invention, the compound of the formula I is administered in combination with bezafibrate and diflunisal.

In a further embodiment of the invention, the compound of the formula I is administered in combination with a solid combination of fenofibrate or a salt thereof with simvastatin, rosuvastatin, fluvastatin, lovastatin, cerivastatin, pravastatin, pitavastatin or atorvastatin.

In a further embodiment of the invention, the compound of the formula I is administered in combination with Synordia®, a solid combination of fenofibrate with metformin.

In one embodiment of the invention, the compound of the formula I is administered in combination with a cholesterol reabsorption inhibitor, for example ezetimibe, tiqueside, pamaqueside, FM-VP4 (sitostanol/campesterol ascorbyl phosphate; Forbes Medi-Tech, WO2005042692, WO2005005453), MD-0727 (Microbia Inc., WO2005021497, WO2005021495) or with compounds as described in WO2002066464, WO2005000353 (Kotobuki Pharmaceutical Co. Ltd.) or WO2005044256 or WO2005062824 (Merck & Co.) or WO2005061451 and WO2005061452 (AstraZeneca AB) and WO2006017257 (Phenomix) or WO2005033100 (Lipideon Biotechnology AG), or as described in WO2002050060, WO2002050068, WO2004000803, WO2004000804, WO2004000805, WO2004087655, WO2004097655, WO2005047248, WO2006086562, WO2006102674, WO2006116499, WO2006121861, WO2006122186, WO2006122216, WO2006127893, WO2006137794, WO2006137796, WO2006137782, WO2006137793, WO2006137797, WO2006137795, WO2006137792, WO2006138163, WO2007059871, US2007232688, WO2007126358, WO2008033431, WO2008033465, WO2008052658, WO2008057336, WO2008085300.

In one embodiment of the invention, the compound of the formula I is administered in combination with an NPC1L1 antagonist, for example those as described in WO2008033464, WO2008033465.

In one embodiment of the invention, the compound of the formula I is administered in combination with Vytorin™, a solid combination of ezetimibe with simvastatin.

In one embodiment of the invention, the compound of the formula I is administered in combination with a solid combination of ezetimibe with atorvastatin.

In one embodiment of invention, the compound of the formula I is administered in combination with a solid combination of ezetimibe with fenofibrate.

In one embodiment of the invention, the further active ingredient is a diphenylazetidinone derivative, as described, for example, in U.S. Pat. No. 6,992,067 or U.S. Pat. No. 7,205,290.

In a further embodiment of the invention, the further active ingredient is a diphenylazetidinone derivative, as described, for example, in U.S. Pat. No. 6,992,067 or U.S. Pat. No. 7,205,290, combined with a statin, for example simvastatin, fluvastatin, pravastatin, lovastatin, cerivastatin, atorvastatin, pitavastatin or rosuvastatin.

In one embodiment of the invention, the compound of the formula I is administered in combination with a solid combination of lapaquistat, a squalene synthase inhibitor, with atorvastatin.

In one embodiment of the invention, the compound of the formula I is administered in combination with a CETP inhibitor, for example torcetrapib, anacetrapib or JTT-705 (dalcetrapib), or those as described in WO2006002342, WO2006010422, WO2006012093, WO2006073973, WO2006072362, WO2007088996, WO2007088999, US2007185058, US2007185113, US2007185154, US2007185182, WO2006097169, WO2007041494, WO2007090752, WO2007107243, WO2007120621, US2007265252, US2007265304, WO2007128568, WO2007132906, WO2008006257, WO2008009435, WO2008018529, WO2008058961, WO2008058967, WO2008059513, WO2008070496, WO2008115442, WO2008111604.

In one embodiment of the invention, the compound of the formula I is administered in combination with bile acid reabsorption inhibitors (inhibitors of the intestinal bile acid transporter (IBAT)) (see, for example, U.S. Pat. No. 6,245,744, U.S. Pat. No. 6,221,897 or WO00/61568), for example HMR 1741, or those as described in DE 10 2005 033099.1 and DE 10 2005 033100.9, DE 10 2006 053635, DE 10 2006 053637, WO2007009655-56, WO2008058628, WO2008058629, WO2008058630, WO2008058631.

In one embodiment, the compound of the formula I is administered in combination with agonists of GPBAR1 (G-protein-coupled bile acid receptor-1; TGR5), as described, for example, in US20060199795, WO2007110237, WO2007127505, WO2008009407, WO2008067219, WO2008067222, FR2908310, WO2008091540, WO2008097976.

In one embodiment, the compound of the formula I is administered in combination with inhibitors of the TRPM5 channel (TRP cation channel M5), as described, for example, in WO2008097504.

In one embodiment of the invention, the compound of the formula I is administered in combination with a polymeric bile acid adsorber, for example cholestyramine, colesevelam hydrochloride.

In one embodiment of the invention, the compound of the formula I is administered in combination with colesevelam hydrochloride and metformin or a sulfonylurea or insulin.

In one embodiment of the invention, the compound of the formula I is administered in combination with a chewing gum comprising phytosterols (Reductol™).

In one embodiment of the invention, the compound of the formula I is administered in combination with an inhibitor of the microsomal triglyceride transfer protein (MTP inhibitor), for example implitapide, BMS-201038, R-103757, AS-1552133, SLx-4090, AEGR-733, or those as described in WO2005085226, WO2005121091, WO2006010423, WO2006113910, WO2007143164, WO2008049806, WO2008049808, WO2008090198, WO2008100423.

In a further embodiment of the invention, the compound of the formula I is administered in combination with a combination of a cholesterol absorption inhibitor, for example ezetimibe, and an inhibitor of the triglyceride transfer protein (MTP inhibitor), for example implitapide, as described in WO2008030382 or in WO2008079398.

In one embodiment of the invention, the compound of the formula I is administered in combination with an active antihypertriglyceridemic ingredient, for example those as described in WO2008032980.

In another embodiment of the invention, the compound of the formula I is administered in combination with an antagonist of the somatostatin 5 receptor (SST5 receptor), for example those as described in WO2006094682.

In one embodiment of the invention, the compound of the formula I is administered in combination with an ACAT inhibitor, for example avasimibe, SMP-797 or KY-382 or those as are described in WO2008087029, WO2008087030, WO2008095189.

In a further embodiment of the invention, the compound of the formula I is administered in combination with an inhibitor of liver carnitine palmitoyltransferase 1 (L-CPT1), as described, for example, in WO2007063012, WO2007096251 (ST-3473), WO2008015081, US2008103182, WO2008074692.

In a further embodiment of the invention, the compound of the formula I is administered in combination with a modulator of serine palmitoyltransferase (SPT), as described, for example, in WO2008031032, WO2008046071, WO2008083280, WO2008084300.

In one embodiment of the invention, the compound of the formula I is administered in combination with a squalene synthetase inhibitor, for example BMS-188494, TAK-475 (lapaquistat acetate), or as described in WO2005077907, JP2007022943, WO2008003424.

In one embodiment of the invention, the compound of the formula I is administered in combination with ISIS-301012 (mipomersen), an antisense oligonucleotide which is capable of regulating the apolipoprotein B gene.

In one embodiment of the invention, the compound of the formula I is administered in combination with a stimulator of the ApoA-1 gene, as described, for example, in WO2008092231.

In one embodiment of the invention, the compound of the formula I is administered in combination with an LDL receptor inducer (see U.S. Pat. No. 6,342,512), for example HMR1171, HMR1586, or those as described in WO2005097738, WO2008020607.

In another embodiment of the invention, the compound of the formula I is administered in combination with an HDL cholesterol-elevating agent, for example those as described in WO2008040651, WO2008099278.

In one embodiment of the invention, the compound of the formula I is administered in combination with an ABCA1 expression enhancer, as described, for example, in WO2006072393, WO2008062830.

In one embodiment of the invention, the compound of the formula I is administered in combination with a lipoproteinlipase modulator, for example ibrolipim (NO-1886).

In one embodiment of the invention, the compound of the formula I is administered in combination with a lipoprotein(a) antagonist, for example gemcabene (CI-1027).

In one embodiment of the invention, the compound of the formula I is administered in combination with a lipase inhibitor, for example orlistat or cetilistat (ATL-962).

In one embodiment of the invention, the compound of the formula I is administered in combination with an adenosine A1 receptor agonist (adenosine A1 R), as described, for example, in EP1258247, EP1375508, WO2008028590, WO2008077050.

In one embodiment of the invention, the compound of the formula I is administered in combination with an adenosine A2B receptor agonist (adenosine A2B R), for example ATL-801.

In another embodiment of the invention, the compound of the formula I is administered in combination with a modulator of adenosine A2A and/or adenosine A3 receptors, as described, for example, in WO2007111954, WO2007121918, WO2007121921, WO2007121923, WO2008070661.

In a further embodiment of the invention, the compound of the formula I is administered in combination with an agonist of the adenosine A1/A2B receptors, as described, for example, in WO2008064788, WO2008064789.

In one embodiment of the invention, the compound of the formula I is administered in combination with an adenosine A2B receptor antagonist (adenosine A2B R), as described in US2007270433, WO2008027585, WO2008080461.

In one embodiment, the compound of the formula I is administered in combination with inhibitors of acetyl-CoA carboxylase (ACC1 and/or ACC2), for example those as described in WO199946262, WO200372197, WO2003072197, WO2005044814, WO2005108370, JP2006131559, WO2007011809, WO2007011811, WO2007013691, WO2007095601-603, WO2007119833, WO2008065508, WO2008069500, WO2008070609, WO2008072850, WO2008079610, WO2008088688, WO2008088689, WO2008088692, US2008171761, WO2008090944, JP2008179621, US2008200461, WO2008102749, WO2008103382, WO2008121592.

In another embodiment, the compound of the formula I is administered in combination with modulators of microsomal acyl-CoA:glycerol-3-phosphate acyltransferase 3 (GPAT3, described in WO2007100789) or with modulators of microsomal acyl-CoA:glycerol-3-phosphate acyltransferase 4 (GPAT4, described in WO2007100833).

In a further embodiment, the compound of the formula I is administered in combination with modulators of xanthine oxidoreductase (XOR).

In another embodiment, the compound of the formula I is administered in combination with inhibitors of soluble epoxide hydrolase (sEH), as described, for example, in WO2008051873, WO2008051875, WO2008073623, WO2008094869, WO2008112022.

In a further embodiment, the compound of the formula I is administered in combination with CART modulators (see “Cocaine-amphetamine-regulated transcript influences energy metabolism, anxiety and gastric emptying in mice” Asakawa, A. et al.: Hormone and Metabolic Research (2001), 33(9), 554-558);

NPY antagonists, for example N-{4-[(4-aminoquinazolin-2-ylamino)methyl]-cyclohexylmethyl}naphthalene-1-sulfonamide hydrochloride (CGP 71683A) or velneperit;
NPY-5 receptor antagonists, such as L-152804 or the compound “NPY-5-BY” from Banyu, or as described, for example, in WO2006001318, WO2007103295, WO2007125952, WO2008026563, WO2008026564, WO2008052769, WO2008092887, WO2008092888, WO2008092891;
NPY-4 receptor antagonists, as described, for example, in WO2007038942;
NPY-2 receptor antagonists, as described, for example, in WO2007038943; peptide YY 3-36 (PYY3-36) or analogous compounds, for example CJC-1682 (PYY3-36 conjugated with human serum albumin via Cys34) or CJC-1643 (derivative of PYY3-36, which is conjugated in vivo to serum albumin), or those as described in WO2005080424, WO2006095166, WO2008003947;
derivatives of the peptide obestatin, as described by WO2006096847;
CB1R (cannabinoid receptor 1) antagonists, for example rimonabant, surinabant (SR147778), SLV-319 (ibipinabant), AVE-1625, taranabant (MK-0364) or salts thereof, otenabant (CP-945,598), rosonabant, V-24343 or those compounds as described in, for example, EP 0656354, WO 00/15609, WO2001/64632-64634, WO 02/076949, WO2005080345, WO2005080328, WO2005080343, WO2005075450, WO2005080357, WO200170700, WO2003026647-48, WO200302776, WO2003040107, WO2003007887, WO2003027069, U.S. Pat. No. 6,509,367, WO200132663, WO2003086288, WO2003087037, WO2004048317, WO2004058145, WO2003084930, WO2003084943, WO2004058744, WO2004013120, WO2004029204, WO2004035566, WO2004058249, WO2004058255, WO2004058727, WO2004069838, US20040214837, US20040214855, US20040214856, WO2004096209, WO2004096763, WO2004096794, WO2005000809, WO2004099157, US20040266845, WO2004110453, WO2004108728, WO2004000817, WO2005000820, US20050009870, WO200500974, WO2004111033-34, WO200411038-39, WO2005016286, WO2005007111, WO2005007628, US20050054679, WO2005027837, WO2005028456, WO2005063761-62, WO2005061509, WO2005077897, WO2006018662, WO2006047516, WO2006060461, WO2006067428, WO2006067443, WO2006087480, WO2006087476, WO2006100208, WO2006106054, WO2006111849, WO2006113704, WO2007009705, WO2007017124, WO2007017126, WO2007018459, WO2007018460, WO2007016460, WO2007020502, WO2007026215, WO2007028849, WO2007031720, WO2007031721, WO2007036945, WO2007038045, WO2007039740, US20070015810, WO2007046548, WO2007047737, WO2007057687, WO2007062193, WO2007064272, WO2007079681, WO2007084319, WO2007084450, WO2007086080, EP1816125, US2007213302, WO2007095513, WO2007096764, US2007254863, WO2007119001, WO2007120454, WO2007121687, WO2007123949, US2007259934, WO2007131219, WO2007133820, WO2007136571, WO2007136607, WO2007136571, U.S. Pat. No. 7,297,710, WO2007138050, WO2007139464, WO2007140385, WO2007140439, WO2007146761, WO2007148061, WO2007148062, US2007293509, WO2008004698, WO2008017381, US2008021031, WO2008024284, WO2008031734, WO2008032164, WO2008034032, WO2008035356, WO2008036021, WO2008036022, WO2008039023, WO2998043544, WO2008044111, WO2008048648, EP1921072-A1, WO2008053341, WO2008056377, WO2008059207, WO2008059335, WO2008062424, WO2008068423, WO2008068424, WO2008070305, WO2008070306, WO2008074816, WO2008074982, WO2008075012, WO2008075013, WO2008075019, WO2008075118, WO2008076754, WO2008081009, WO2008084057, EP1944295, US2008090809, US2008090810, WO2008092816, WO2008094473, WO2008094476, WO2008099076, WO2008099139, WO2008101995, US2008207704, WO2008107179, WO2008109027, WO2008112674, WO2008115705, WO2008118414, WO2008119999, WO200812000, WO2008121257, WO2008127585;
cannabinoid receptor 1/cannabinoid receptor 2 (CB1/CB2) modulating compounds, for example delta-9-tetrahydrocannabivarin, or those as described, for example, in WO2007001939, WO2007044215, WO2007047737, WO2007095513, WO2007096764, WO2007112399, WO2007112402, WO2008122618;
modulators of FAAH (fatty acid amide hydrolase), as described, for example, in WO2007140005, WO2008019357, WO2008021625, WO2008023720, WO2008030532;
inhibitors of fatty acid synthase (FAS), as described, for example, in WO2008057585, WO2008059214, WO2008075064, WO2008075070, WO2008075077;
inhibitors of LCE (long chain fatty acid elongase), as described, for example, in WO2008120653;
vanilloid-1 receptor modulators (modulators of TRPV1), as described, for example, in WO2007091948, WO2007129188, WO2007133637, WO2008007780, WO2008010061, WO2008007211, WO2008010061, WO2008015335, WO2008018827, WO2008024433, WO2008024438, WO2008032204, WO2008050199, WO2008059339, WO2008059370, WO2008066664, WO2008075150, WO2008090382, WO2008090434, WO2008093024, WO2008107543, WO2008107544, WO2008110863;
modulators, antagonists or inverse agonists of the opioid receptors, for example GSK-982 or those as described, for example, in WO2007047397, WO2008021849, WO2008021851, WO2008032156, WO2008059335;
modulators of the orphan opioid (ORL-1) receptor, as described, for example, in US2008249122, WO2008089201;
agonists of the prostaglandin receptor, for example bimatoprost or those compounds as described in WO2007111806;
MC4 receptor agonists (melanocortin-4 receptor agonists, MC4R agonists, for example N-[2-(3a-benzyl-2-methyl-3-oxo-2,3,3a,4,6,7-hexahydropyrazolo[4,3-c]-pyridin-5-yl)-1-(4-chlorophenyl)-2-oxoethyl]-1-amino-1,2,3,4-tetrahydronaphthalene-2-carboxamide; (WO 01/91752)) or LB53280, LB53279, LB53278 or THIQ, MB243, RY 764, CHIR-785, PT-141, MK-0493, or those as described in WO2005060985, WO2005009950, WO2004087159, WO2004078717, WO2004078716, WO2004024720, US20050124652, WO2005051391, WO2004112793, WOUS20050222014, US20050176728, US20050164914, US20050124636, US20050130988, US20040167201, WO2004005324, WO2004037797, WO2005042516, WO2005040109, WO2005030797, US20040224901, WO200501921, WO200509184, WO2005000339, EP1460069, WO2005047253, WO2005047251, WO2005118573, EP1538159, WO2004072076, WO2004072077, WO2006021655-57, WO2007009894, WO2007015162, WO2007041061, WO2007041052, JP2007131570, EP-1842846, WO2007096186, WO2007096763, WO2007141343, WO2008007930, WO2008017852, WO2008039418, WO2008087186, WO2008087187, WO2008087189, WO2008087186-WO2008087190, WO2008090357;
orexin receptor 1 antagonists (OX1R antagonists), orexin receptor 2 antagonists (OX2R antagonists) or mixed OX1R/OX2R antagonists (e.g. 1-(2-methyl-benzoxazol-6-yl)-3-[1,5]naphthyridin-4-ylurea hydrochloride (SB-334867-A), or those as described, for example, in WO200196302, WO200185693, WO2004085403, WO2005075458, WO2006067224, WO2007085718, WO2007088276, WO2007116374, WO2007122591, WO2007126934, WO2007126935, WO2008008517, WO2008008518, WO2008008551, WO2008020405, WO2008026149, WO2008038251, US2008132490, WO2008065626, WO2008078291, WO2008087611, WO2008081399, WO2008108991, WO2008107335, US2008249125);
histamine H3 receptor antagonists/inverse agonists (e.g. 3-cyclohexyl-1-(4,4-dimethyl-1,4,6,7-tetrahydroimidazo[4,5-c]pyridin-5-yl)propan-1-one oxalic acid salt (WO 00/63208), or those as described in WO200064884, WO2005082893, US2005171181 (e.g. PF-00389027), WO2006107661, WO2007003804, WO2007016496, WO2007020213, WO2007049798, WO2007055418, WO2007057329; WO2007065820, WO2007068620, WO2007068641, WO2007075629, WO2007080140, WO2007082840, WO2007088450, WO2007088462, WO2007094962, WO2007099423, WO2007100990, WO2007105053, WO2007106349, WO2007110364, WO2007115938, WO2007131907, WO2007133561, US2007270440, WO2007135111, WO2007137955, US2007281923, WO2007137968, WO2007138431, WO2007146122, WO2008005338, WO2008012010, WO2008015125, WO2008045371, EP1757594, WO2008068173, WO2008068174, US20080171753, WO2008072703, WO2008072724, US2008188484, US2008188486, US2008188487, WO2008109333, WO2008109336);
histamine H1/histamine H3 modulators, for example betahistine or its dihydrochloride;
modulators of the histamine H3 transporter or of the histamine H3/serotonin transporter, as described, for example, in WO2008002816, WO2008002817, WO2008002818, WO2008002820;
histamine H4 modulators, as described, for example, in WO2007117399;
CRF antagonists (e.g. [2-methyl-9-(2,4,6-trimethylphenyl)-9H-1,3,9-triazafluoren-4-yl]dipropylamine (WO 00/66585) or those CRF1 antagonists as described in WO2007105113, WO2007133756, WO2008036541, WO2008036579, WO2008083070);
CRF BP antagonists (e.g. urocortin);
urocortin agonists;
modulators of the beta-3 adrenoceptor, for example 1-(4-chloro-3-methanesulfonylmethylphenyl)-2-[2-(2,3-dimethyl-1H-indol-6-yloxy)ethylamino]-ethanol hydrochloride (WO 01/83451) or solabegron (GW-427353) or N-5984 (KRP-204), or those as described in JP2006111553, WO2002038543, WO2002038544, WO2007048840-843, WO2008015558, EP1947103;
MSH (melanocyte-stimulating hormone) agonists;
MCH (melanine-concentrating hormone) receptor antagonists (for example NBI-845, A-761, A-665798, A-798, ATC-0175, T-226296, T-71 (AMG-071, AMG-076), GW-856464, NGD-4715, ATC-0453, ATC-0759, GW-803430 or those compounds as described in WO2005085200, WO2005019240, WO2004011438, WO2004012648, WO2003015769, WO2004072025, WO2005070898, WO2005070925, WO2004039780, WO2004092181, WO2003033476, WO2002006245, WO2002089729, WO2002002744, WO2003004027, FR2868780, WO2006010446, WO2006038680, WO2006044293, WO2006044174, JP2006176443, WO2006018280, WO2006018279, WO2006118320, WO2006130075, WO2007018248, WO2007012661, WO2007029847, WO2007024004, WO2007039462, WO2007042660, WO2007042668, WO2007042669, US2007093508, US2007093509, WO2007048802, JP2007091649, WO2007092416; WO2007093363-366, WO2007114902, WO2007114916, WO2007141200, WO2007142217, US2007299062, WO2007146758, WO2007146759, WO200800116, WO2008016811, WO2008020799, WO2008022979, WO2008038692, WO2008041090, WO2008044632, WO2008047544, WO2008061109, WO2008065021, WO2008068265, WO2008071646, WO2008076562, JP2008088120, WO2008086404, WO2008086409);
CCK-A (CCK-1) agonists/modulators (for example {2-[4-(4-chloro-2,5-dimethoxyphenyl)-5-(2-cyclohexylethyl)thiazol-2-ylcarbamoyl]-5,7-dimethylindol-1-yl}acetic acid trifluoro-acetic acid salt (WO 99/15525) or SR-146131 (WO 0244150) or SSR-125180), or those as described in WO2005116034, WO2007120655, WO2007120688, WO2007120718, WO2008091631;
serotonin reuptake inhibitors (e.g. dexfenfluramine), or those as described in WO2007148341, WO2008034142, WO2008081477, WO2008120761;
mixed serotonin/dopamine reuptake inhibitors (e.g. bupropion), or those as described in WO2008063673, or solid combinations of bupropion with naltrexone or bupropion with zonisamide;
mixed reuptake inhibitors, for example DOV-21947;
mixed serotoninergic and noradrenergic compounds (e.g. WO 00/71549);
5-HT receptor agonists, for example 1-(3-ethylbenzofuran-7-yl)piperazine oxalic acid salt (WO 01/09111);
mixed dopamine/norepinephrine/acetylcholine reuptake inhibitors (e.g. tesofensine), or those as described, for example, in WO2006085118;
dopamine antagonists as described, for example, in WO2008079838, WO2008079839, WO2008079847, WO2008079848;
norepinephrine reuptake inhibitors, as described, for example, in US2008076724;
5-HT2A receptor antagonists, as described, for example, in WO2007138343;
5-HT2C receptor agonists (for example lorcaserine hydrochloride (APD-356) or BVT-933, or those as described in WO200077010, WO200077001-02, WO2005019180, WO2003064423, WO200242304, WO2005035533, WO2005082859, WO2006004937, US2006025601, WO2006028961, WO2006077025, WO2006103511, WO2007028132, WO2007084622, US2007249709; WO2007132841, WO2007140213, WO2008007661, WO2008007664, WO2008009125, WO2008010073, WO2008108445);
5-HT6 receptor modulators, for example E-6837, BVT-74316 or PRX-07034, or those as described, for example, in WO2005058858, WO2007054257, WO2007107373, WO2007108569, WO2007108742-744, WO2008003703, WO2008027073, WO2008034815, WO2008054288, EP1947085, WO2008084491, WO2008084492, WO2008092665, WO2008092666, WO2008101247, WO2008110598, WO2008116831, WO2008116833; agonists of estrogen receptor gamma (ERRγ agonists), as described, for example, in WO2007131005, WO2008052709;
agonists of estrogen receptor alpha (ERRα/ERR1 agonists), as described, for example, in WO2008109727;
sigma-1 receptor antagonists, as described, for example, in WO2007098953, WO2007098961, WO2008015266, WO2008055932, WO2008055933;
muscarin 3 receptor (M3R) antagonists, as described, for example, in WO2007110782, WO2008041184;
bombesin receptor agonists (BRS-3 agonists), as described, for example, in WO2008051404, WO2008051405, WO2008051406, WO2008073311;
galanin receptor antagonists;
growth hormone (e.g. human growth hormone or AOD-9604);
growth hormone releasing compounds (tert-butyl 6-benzyloxy-1-(2-diisopropylaminoethylcarbamoyl)-3,4-dihydro-1H-isoquinoline-2-carboxylate (WO 01/85695));
growth hormone secretagogue receptor antagonists (ghrelin antagonists), for example A-778193, or those as described in WO2005030734, WO2007127457, WO2008008286;
growth hormone secretagogue receptor modulators (ghrelin modulators), for example JMV-2959, JMV-3002, JMV-2810, JMV-2951, or those as described in WO2006012577 (e.g. YIL-781 or YIL-870), WO2007079239, WO2008092681;
TRH agonists (see, for example, EP 0 462 884);
decoupling protein 2 or 3 modulators;
chemical decouplers (for example, WO2008059023, WO2008059024, WO2008059025, WO2008059026);
leptin agonists (see, for example, Lee, Daniel W.; Leinung, Matthew C.; Rozhayskaya-Arena, Marina; Grasso, Patricia. Leptin agonists as a potential approach to the treatment of obesity. Drugs of the Future (2001), 26(9), 873-881);
DA agonists (bromocriptin, doprexin);
lipase/amylase inhibitors (e.g. WO 00/40569, WO2008107184);
inhibitors of diacylglycerol O-acyltransferases (DGATs), for example BAY-74-4113, or as described, for example, in US2004/0224997, WO2004094618, WO200058491, WO2005044250, WO2005072740, JP2005206492, WO2005013907, WO2006004200, WO2006019020, WO2006064189, WO2006082952, WO2006120125, WO2006113919, WO2006134317, WO2007016538, WO2007060140, JP2007131584, WO2007071966, WO2007126957, WO2007137103, WO2007137107, WO2007138304, WO2007138311, WO2007141502, WO2007141517, WO2007141538, WO2007141545, WO2007144571, WO2008011130, WO2008011131, WO2008039007, WO2008048991, WO2008067257, WO2008099221;
inhibitors of monoacylglycerol acyltransferase (2-acylglycerol O-acyltransferase; MGAT), as described, for example, in WO2008038768;
inhibitors of fatty acid synthase (FAS), for example C75, or those as described in WO2004005277, WO2008006113;
inhibitors of stearoyl-CoA delta9 desaturase (SCD1), as described, for example, in WO2007009236, WO2007044085, WO2007046867, WO2007046868, WO20070501124, WO2007056846, WO2007071023, WO2007130075, WO2007134457, WO2007136746, WO2007143597, WO2007143823, WO2007143824, WO2008003753, WO2008017161, WO2008024390, WO2008029266, WO2008036715, WO2008043087, WO2008044767, WO2008046226, WO2008056687, WO2008062276, WO2008064474, WO2008074824, WO2008074832, WO2008074833, WO2008074834, WO2008074835, WO2008089580, WO2008096746, WO2008104524, WO2008116898, US2008249100, WO2008120744, WO2008120759, WO2008123469, WO2008127349;
inhibitors of fatty acid desaturase-1 (delta5 desaturase), as described, for example, in WO2008089310; hypoglycemic/hypertriglyceridemic indoline compounds, as described in WO2008039087;
inhibitors of “adipocyte fatty acid-binding protein aP2”, for example BMS-309403;
activators of adiponectin secretion, as described, for example, in WO2006082978, WO2008105533;
promoters of adiponectin production, as described, for example, in WO2007125946, WO2008038712;
modified adiponectins, as described, for example, in WO2008121009; oxyntomodulin or analogs thereof;
oleoyl-estrone
or agonists or partial agonists of the thyroid hormone receptor (thyroid hormone receptor agonists), for example: KB-2115 (eprotirome), QRX-431 (sobetirome) or DITPA, or those as described in WO20058279, WO200172692, WO200194293, WO2003084915, WO2004018421, WO2005092316, WO2007003419, WO2007009913, WO2007039125, WO2007110225, WO2007110226, WO2007128492, WO2007132475, WO2007134864, WO2008001959, WO2008106213;
or agonists of the thyroid hormone receptor beta (TR-beta), for example MB-07811 or MB-07344, or those as described in WO2008062469.

In one embodiment of the invention, the compound of the formula I is administered in combination with a combination of epotirome with ezetimibe.

In one embodiment of the invention, the compound of the formula I is administered in combination with an inhibitor of site-1 protease (S1P), for example PF-429242.

In a further embodiment of the invention, the compound of the formula I is administered in combination with a modulator of trace amine-associated receptor 1 (TAAR1), as described, for example, in US2008146523, WO2008092785.

In one embodiment of the invention, the compound of the formula I is administered in combination with an inhibitor of growth factor receptor-bound protein 2 (GRB2), as described, for example, in WO2008067270.

In a further embodiment of the invention, the compound of the formula I is administered in combination with an RNAi (siRNA) therapeutic agent directed against PCSK9 (proprotein convertase subtilisin/kexin type 9).

In one embodiment, the compound of the formula I is administered in combination with Omacor® or Lovaza™ (omega-3 fatty acid ester; highly concentrated ethyl ester of eicosapentaenoic acid and of docosahexaenoic acid).

In one embodiment, the compound of the formula I is administered in combination with lycopene.

In one embodiment of the invention, the compound of the formula I is administered in combination with an antioxidant, for example OPC-14117, AGI-1067 (succinobucol), probucol, tocopherol, ascorbic acid, β-carotene or selenium.

In one embodiment of the invention, the compound of the formula I is administered in combination with a vitamin, for example Vitamin B6 or Vitamin B12.

In one embodiment, the compound of the formula I is administered in combination with more than one of the aforementioned compounds, for example in combination with a sulfonylurea and metformin, a sulfonylurea and acarbose, repaglinide and metformin (PrandiMet™), insulin and a sulfonylurea, insulin and metformin, insulin and troglitazone, insulin and lovastatin, etc.

In another embodiment, the compound of the formula I is administered in combination with an inhibitor of carboanhydrase type 2 (carbonic anhydrase type 2), for example those as described in WO2007065948.

In another embodiment, the compound of the formula I is administered in combination with topiramat or a derivative thereof, as described in WO2008027557.

In a further embodiment, the compound of the formula I is administered in combination with a solid combination of topiramat with phentermine (Qnexa™).

In a further embodiment, the compound of the formula I is administered in combination with an antisense compound, e.g. ISIS-377131, which inhibits the production of the glucocorticoid receptor.

In another embodiment, the compound of the formula I is administered in combination with an aldosterone synthase inhibitor and an antagonist of the glucocorticoid receptor, a cortisol synthesis inhibitor and/or an antagonist of the corticotropin releasing factor, as described, for example, in EP1886695, WO2008119744.

In one embodiment, the compound of the formula I is administered in combination with an agonist of the RUP3 receptor, as described, for example, in WO2007035355, WO2008005576.

In another embodiment, the compound of the formula I is administered in combination with an activator of the gene which codes for ataxia telangiectasia mutated (ATM) protein kinase, for example chloroquine.

In one embodiment, the compound of the formula I is administered in combination with a tau protein kinase 1 inhibitor (TPK1 inhibitor), as described, for example, in WO2007119463.

In one embodiment, the compound of the formula I is administered in combination with a “c-Jun N-terminal kinase” inhibitor (JNK inhibitor), as described, for example, in WO2007125405, WO2008028860, WO2008118626.

In one embodiment, the compound of the formula I is administered in combination with an endothelin A receptor antagonist, for example avosentan (SPP-301).

In one embodiment, the compound of the formula I is administered in combination with modulators of the glucocorticoid receptor (GR), for example KB-3305 or those compounds as described, for example, in WO2005090336, WO2006071609, WO2006135826, WO2007105766, WO2008120661.

In one embodiment, the further active ingredient is varenicline tartrate, a partial agonist of the alpha 4-beta 2 nicotinic acetylcholine receptor.

In one embodiment, the further active ingredient is trodusquemine.

In one embodiment, the further active ingredient is a modulator of the enzyme SIRT1 and/or SIRT3 (an NAD⁺-dependent protein deacetylase); this active ingredient may, for example, be resveratrol in suitable formulations, or those compounds as specified in WO2007019416 (e.g. SRT-1720), WO2008073451.

In one embodiment of the invention, the further active ingredient is DM-71 (N-acetyl-L-cysteine with bethanechol).

In one embodiment, the compound of the formula I is administered in combination with antihypercholesterolemic compounds, as described, for example, in WO2007107587, WO2007111994, WO2008106600, WO2008113796.

In a further embodiment, the compound of the formula I is administered in combination with inhibitors of SREBP (sterol regulatory element-binding protein), as described, for example, in WO2008097835.

In another embodiment, the compound of the formula I is administered in combination with a cyclic peptide agonist of the VPAC2 receptor, as described, for example, in WO2007101146, WO2007133828.

In a further embodiment, the compound of the formula I is administered in combination with an agonist of the endothelin receptor, as described, for example, in WO2007112069.

In a further embodiment, the compound of the formula I is administered in combination with AKP-020 (bis(ethylmaltolato)oxovanadium(IV)).

In another embodiment, the compound of the formula I is administered in combination with tissue-selective androgen receptor modulators (SARM), as described, for example, in WO2007099200, WO2007137874.

In a further embodiment, the compound of the formula I is administered in combination with an AGE (advanced glycation endproduct) inhibitor, as described, for example, in JP2008024673.

In one embodiment of the invention, the further active ingredient is leptin; see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In another embodiment of the invention, the further active ingredient is metreleptin (recombinant methionyl-leptin) combined with pramlintide.

In a further embodiment of the invention, the further active ingredient is the tetrapeptide ISF-402.

In one embodiment, the further active ingredient is dexamphetamine or amphetamine.

In one embodiment, the further active ingredient is fenfluramine or dexfenfluramine.

In another embodiment, the further active ingredient is sibutramine or those derivatives as described in WO2008034142.

In one embodiment, the further active ingredient is mazindol or phentermine.

In a further embodiment, the further active ingredient is geniposidic acid (WO2007100104) or derivatives thereof (JP2008106008).

In one embodiment, the further active ingredient is a nasal calcium channel blocker, for example diltiazem, or those as described in U.S. Pat. No. 7,138,107.

In one embodiment, the further active ingredient is an inhibitor of sodium-calcium ion exchange, for example those as described in WO2008028958, WO2008085711.

In a further embodiment, the further active ingredient is a blocker of calcium channels, for example of CaV3.2 or CaV2.2, as described in WO2008033431, WO2008033447, WO2008033356, WO2008033460, WO2008033464, WO2008033465, WO2008033468, WO2008073461.

In one embodiment, the further active ingredient is a modulator of a calcium channel, for example those as described in WO2008073934, WO2008073936.

In one embodiment, the further active ingredient is a blocker of the “T-type calcium channel”, as described, for example, in WO2008033431, WO2008110008.

In one embodiment, the further active ingredient is an inhibitor of KCNQ potassium channel 2 or 3, for example those as described in US2008027049, US2008027090.

In one embodiment, the further active ingredient is an inhibitor of the potassium Kv1.3 ion channel, for example those as described in WO2008040057, WO2008040058, WO2008046065.

In another embodiment, the further active ingredient is a modulator of the MCP-1 receptor (monocyte chemoattractant protein-1 (MCP-1)), for example those as described in WO2008014360, WO2008014381.

In one embodiment, the further active ingredient is a modulator of somatostatin receptor 5 (SSTR5), for example those as described in WO2008019967, US2008064697, US2008249101, WO2008000692.

In one embodiment, the further active ingredient is a modulator of somatostatin receptor 2 (SSTR2), for example those as described in WO2008051272.

In one embodiment, the further active ingredient is an erythropoietin-mimetic peptide which acts as an erythropoietin (EPO) receptor agonist. Such molecules are described, for example, in WO2008042800.

In a further embodiment, the further active ingredient is an anorectic/a hypoglycemic compound, for example those as described in WO2008035305, WO2008035306, WO2008035686.

In one embodiment, the further active ingredient is an inductor of lipoic acid synthetase, for example those as described in WO2008036966, WO2008036967.

In one embodiment, the further active ingredient is a stimulator of endothelial nitric oxide synthase (eNOS), for example those as described in WO2008058641, WO2008074413.

In one embodiment, the further active ingredient is a modulator of carbohydrate and/or lipid metabolism, for example those as described in WO2008059023, WO2008059024, WO2008059025, WO2008059026.

In a further embodiment, the further active ingredient is an angiotensin II receptor antagonist, for example those as described in WO2008062905, WO2008067378, WO2008062905.

In one embodiment, the further active ingredient is an agonist of the sphingosine-1-phosphate receptor (S1P), for example those as described in WO2008064315, WO2008074820, WO2008074821.

In one embodiment, the further active ingredient is an agent which retards gastric emptying, for example 4-hydroxyisoleucine (WO2008044770).

In one embodiment, the further active ingredient is a muscle-relaxing substance, as described, for example, in WO2008090200.

In a further embodiment, the further active ingredient is an inhibitor of monoamine oxidase B (MAO-B), for example those as described in WO2008092091.

In another embodiment, the further active ingredient is an inhibitor of the binding of cholesterol and/or triglycerides to the SCP-2 protein (sterol carrier protein-2), for example those as described in US2008194658.

In another embodiment, the further active ingredient is lisofylline, which prevents autoimmune damage to insulin-producing cells.

In one embodiment, the compound of the formula I is administered in combination with bulking agents, preferably insoluble bulking agents (see, for example, Carob/Caromax® (Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Höchst, 65926 Frankfurt/Main). Combination with Caromax® is possible in one preparation or by separate administration of compounds of the formula I and Caromax®. Caromax® can in this connection also be administered in the form of food products such as, for example, in bakery products or muesli bars.

It will be appreciated that every suitable combination of the compounds of the invention with one or more of the aforementioned compounds and optionally one or more other pharmacologically active substances is regarded as falling within the protection conferred by the present invention.

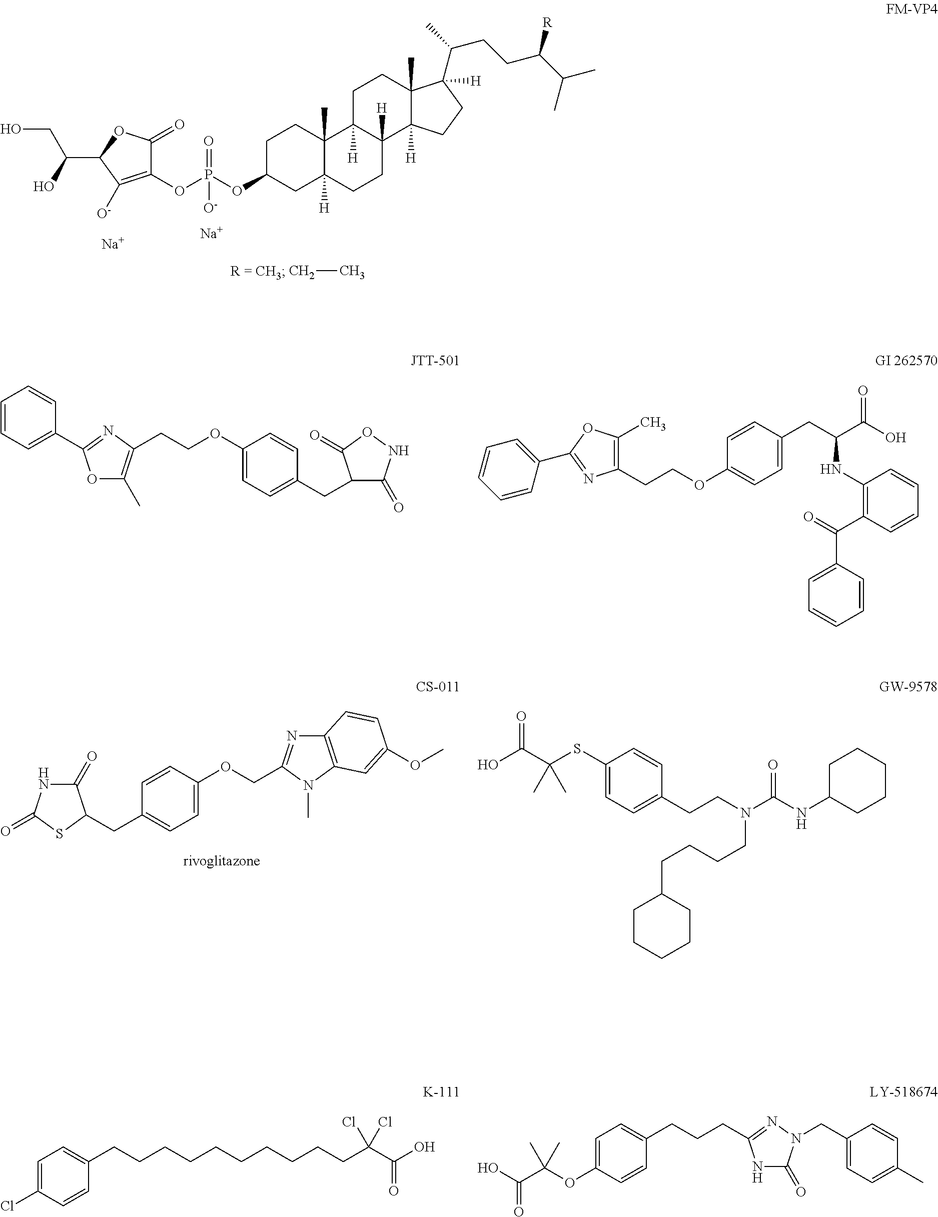

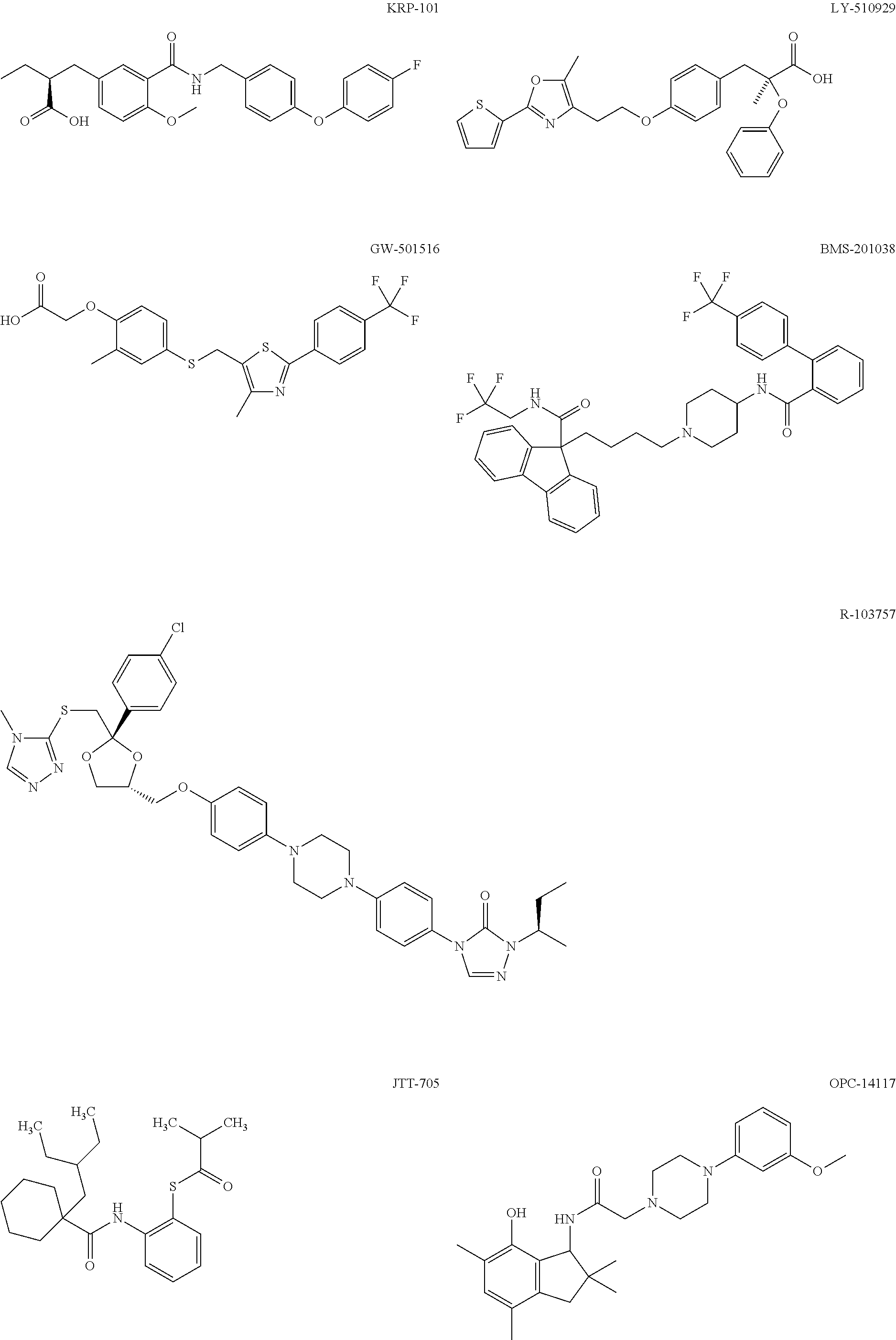

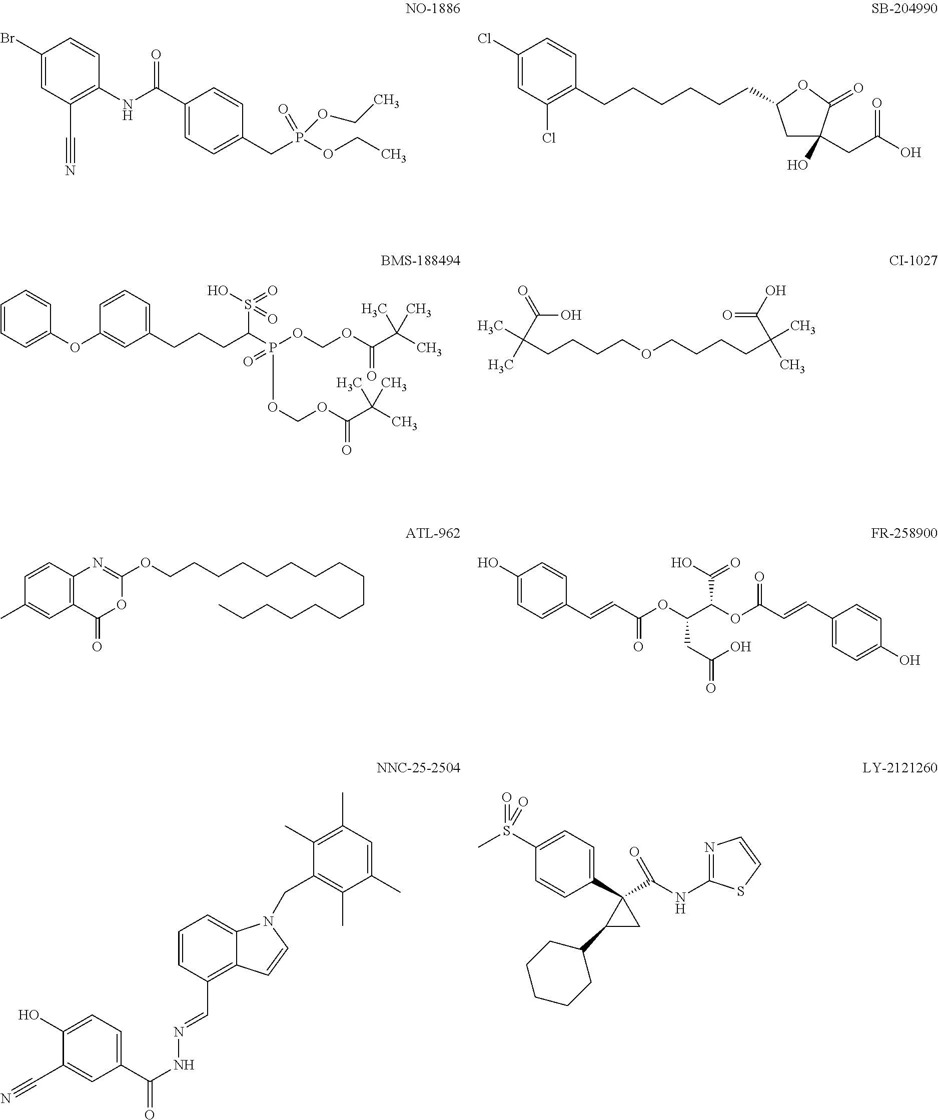

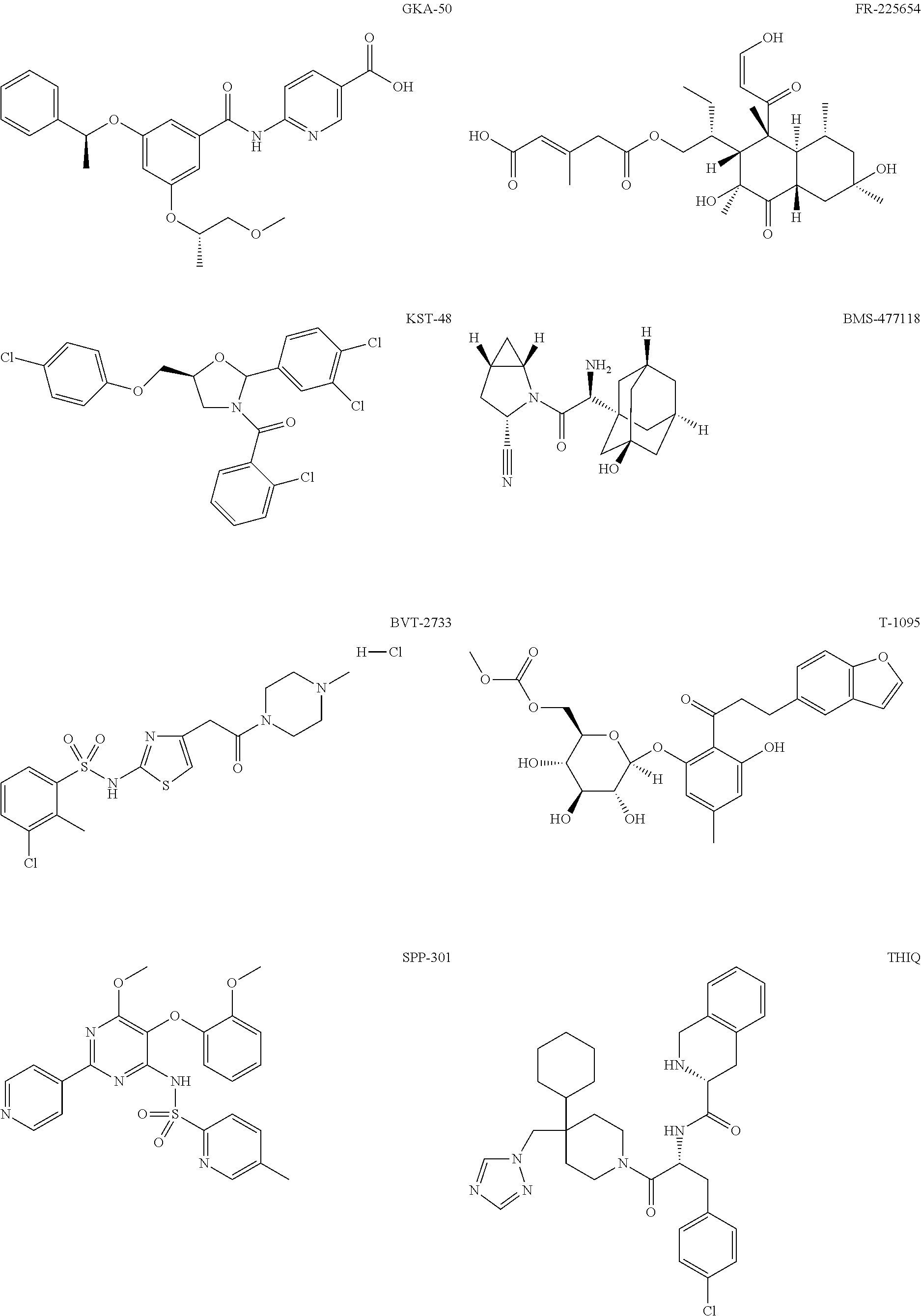

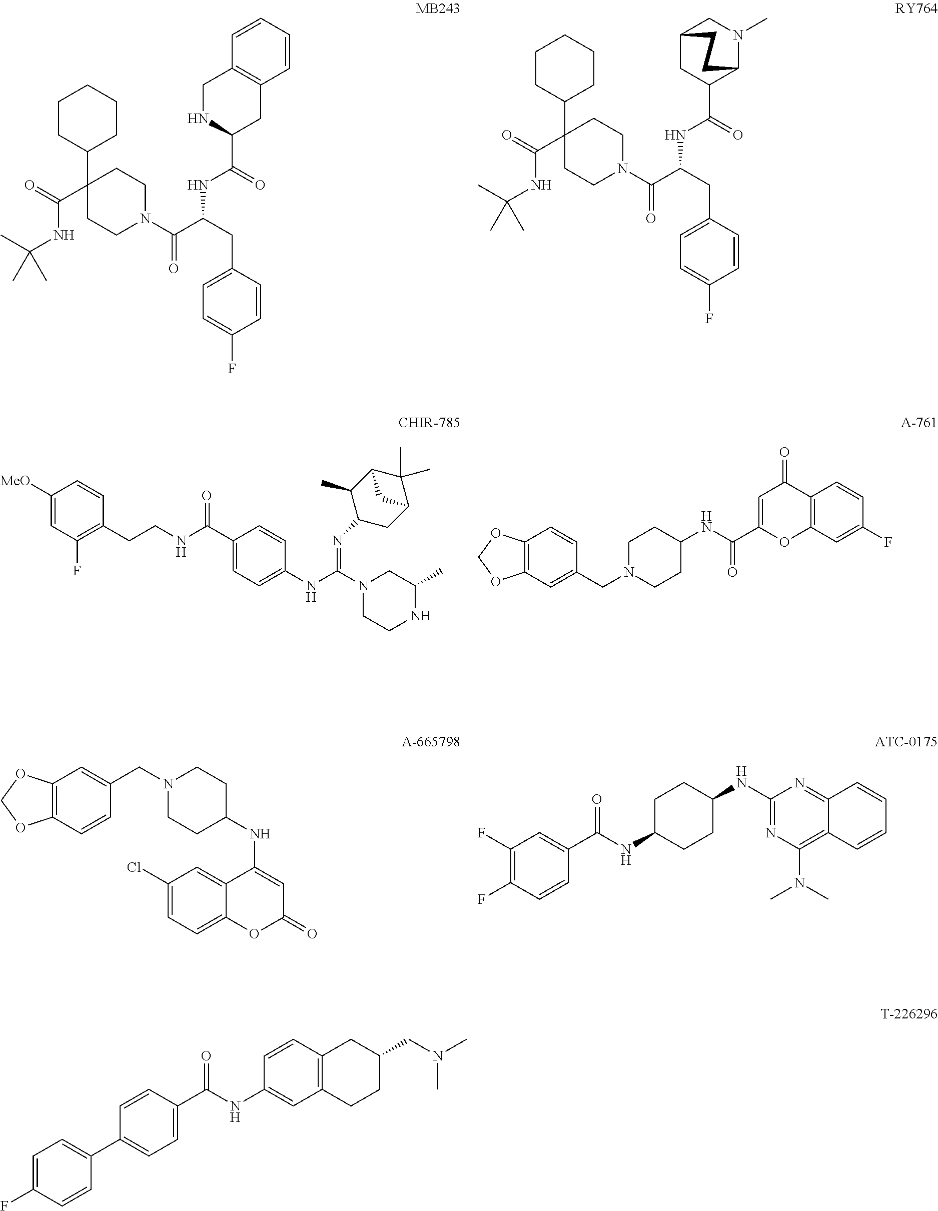

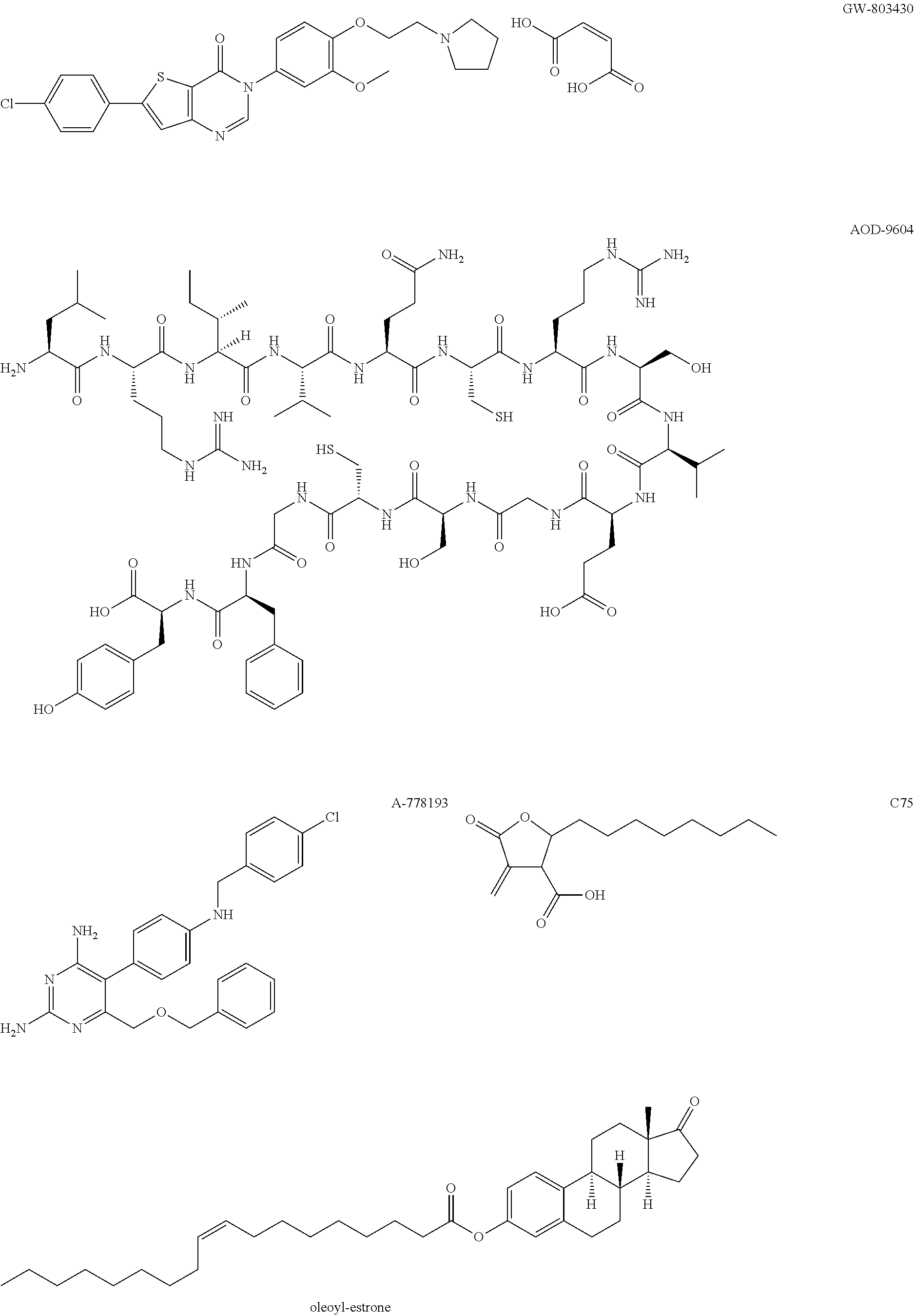

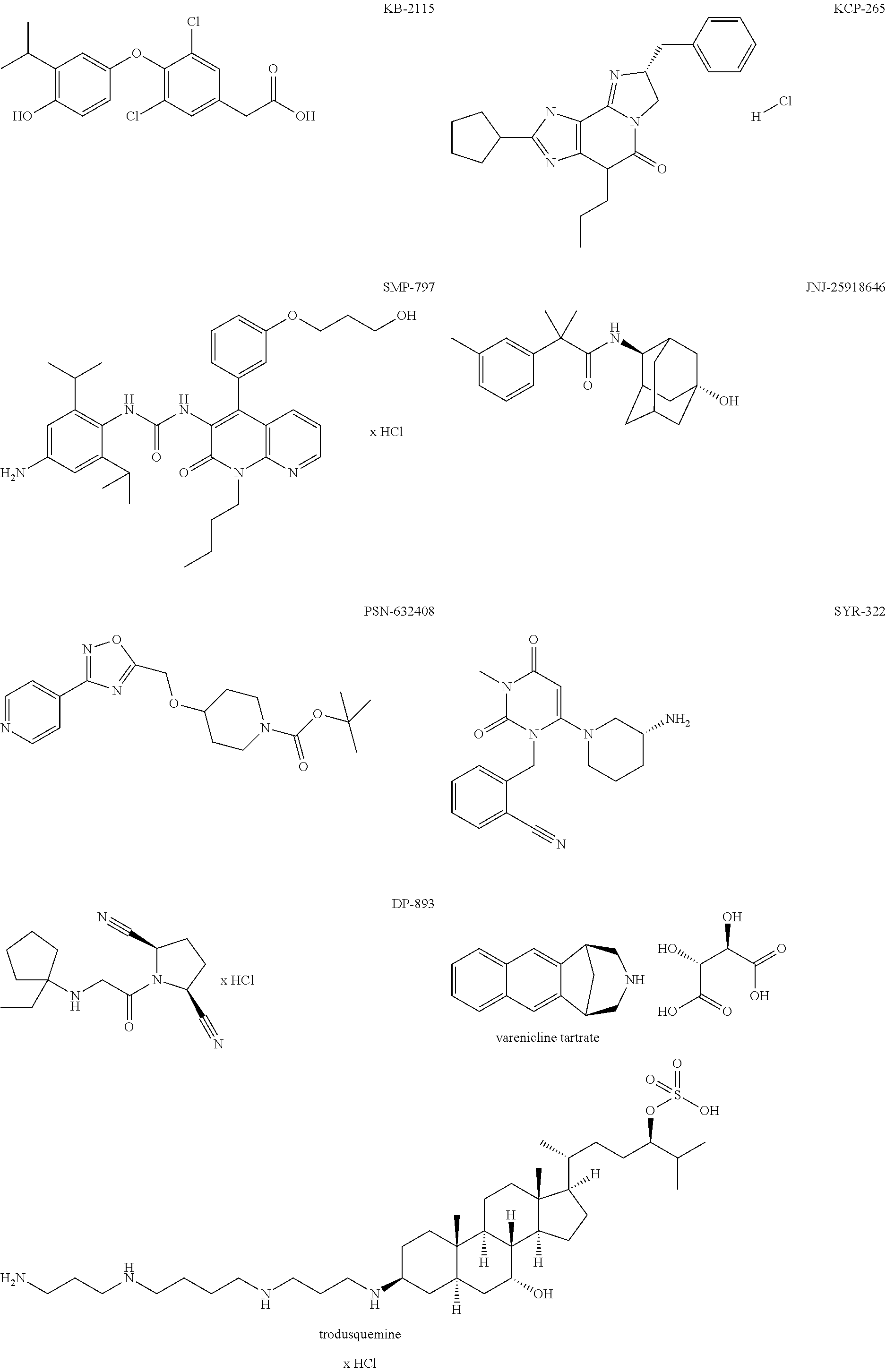

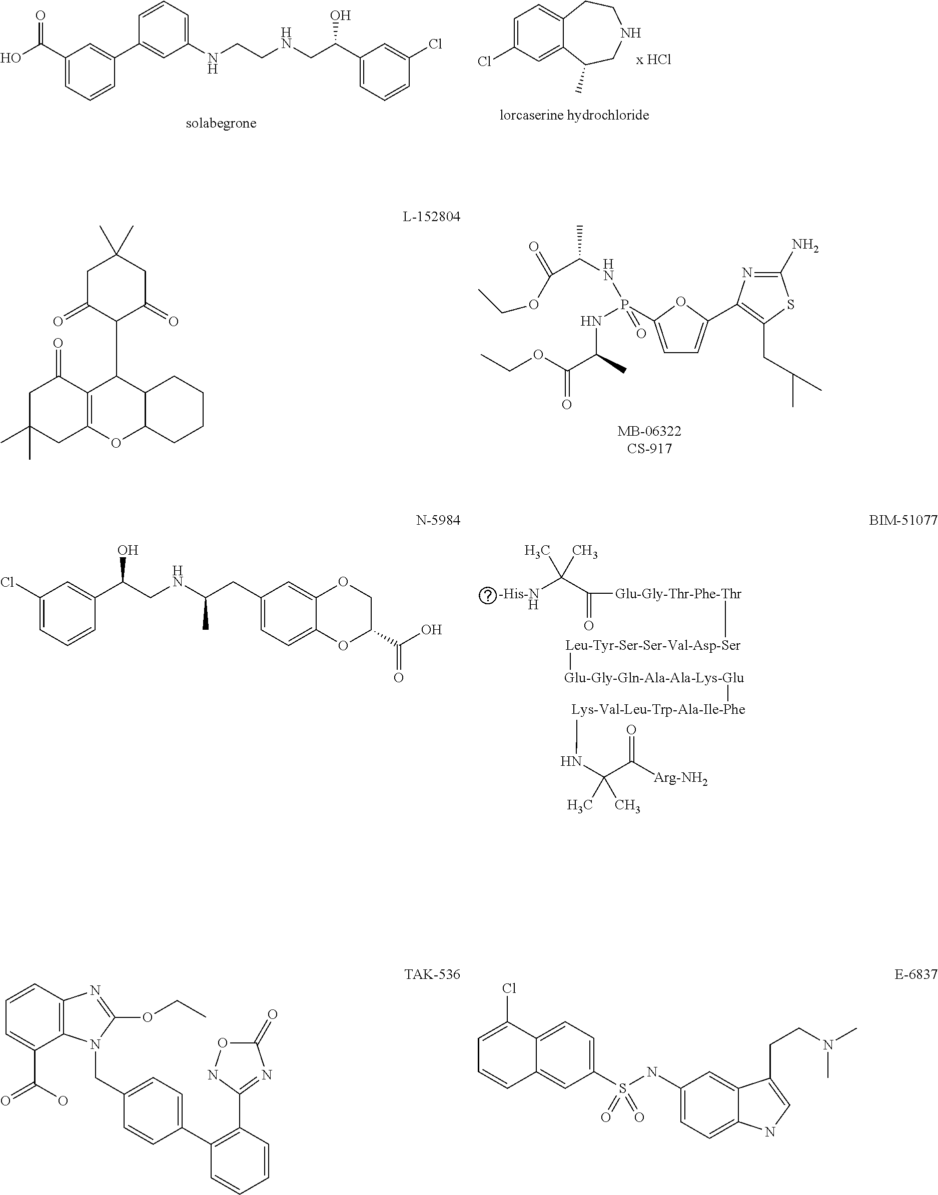

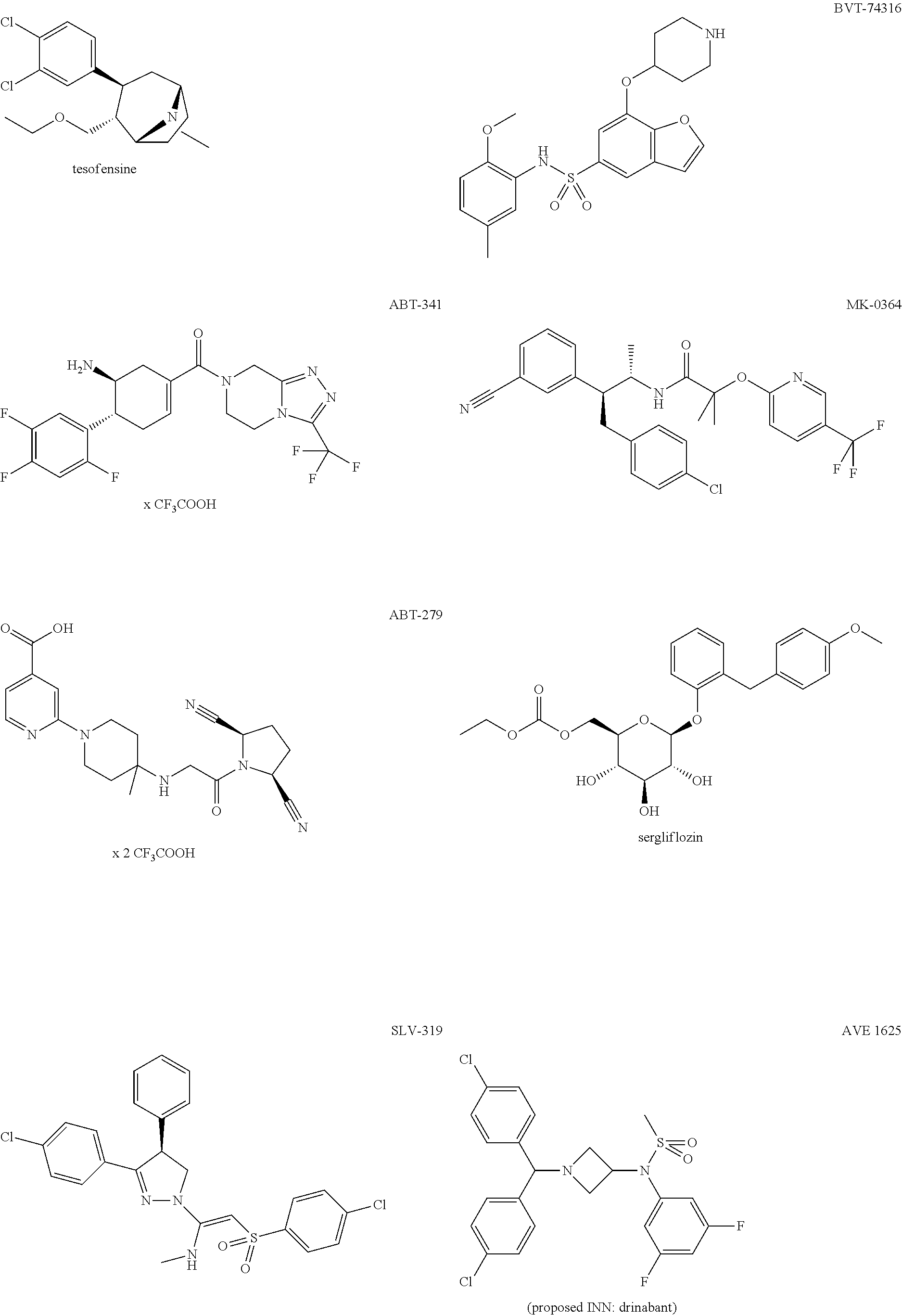

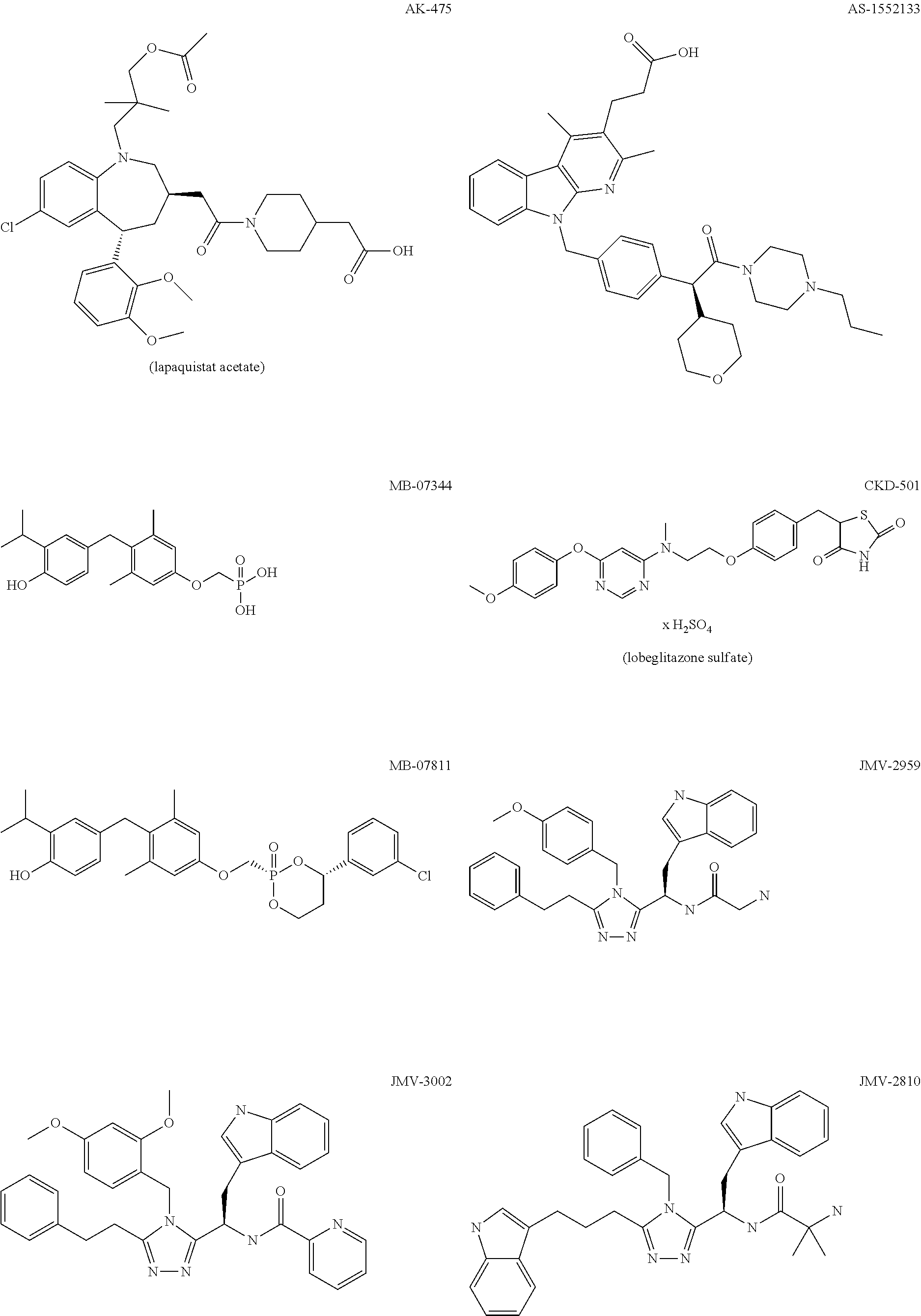

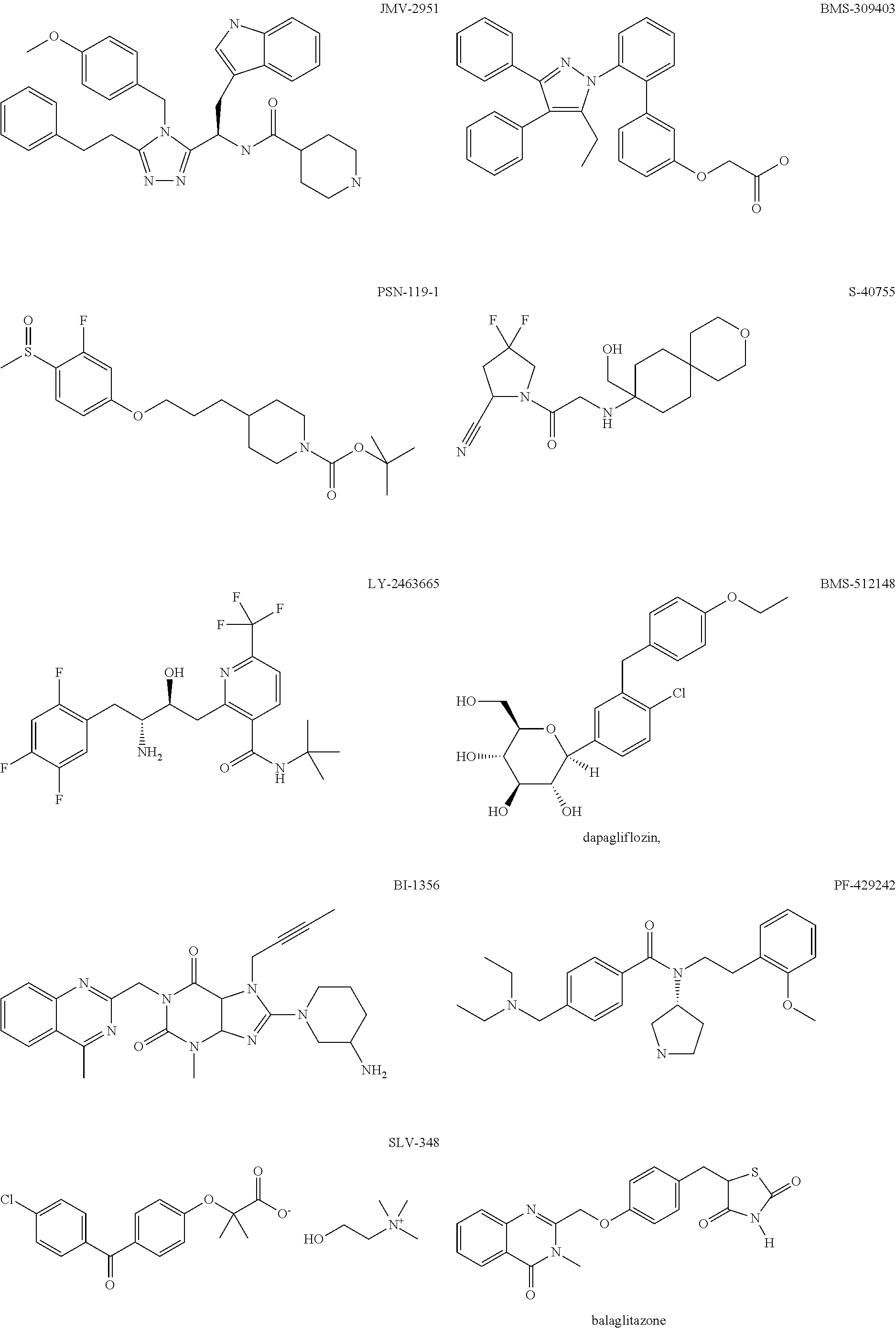

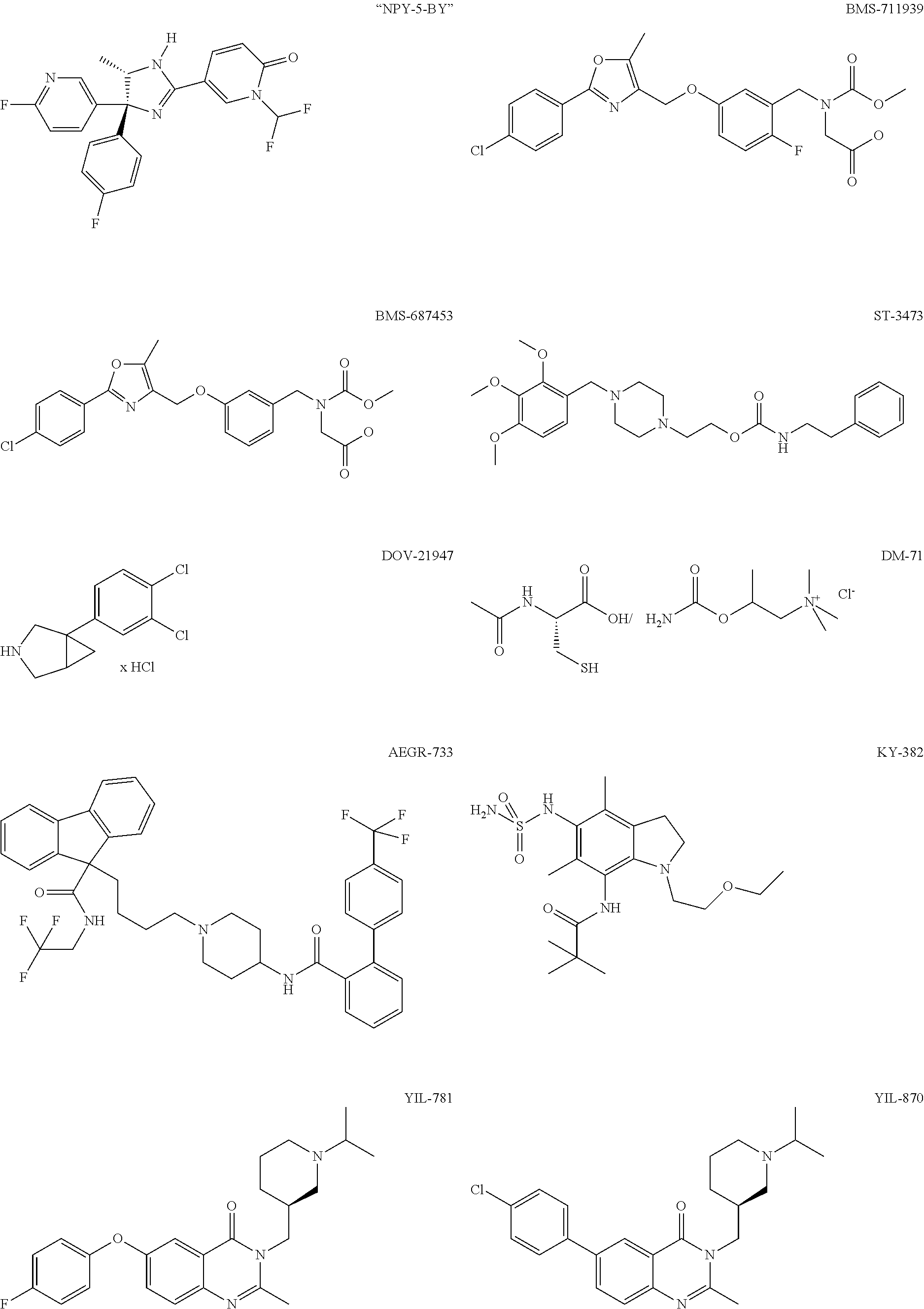

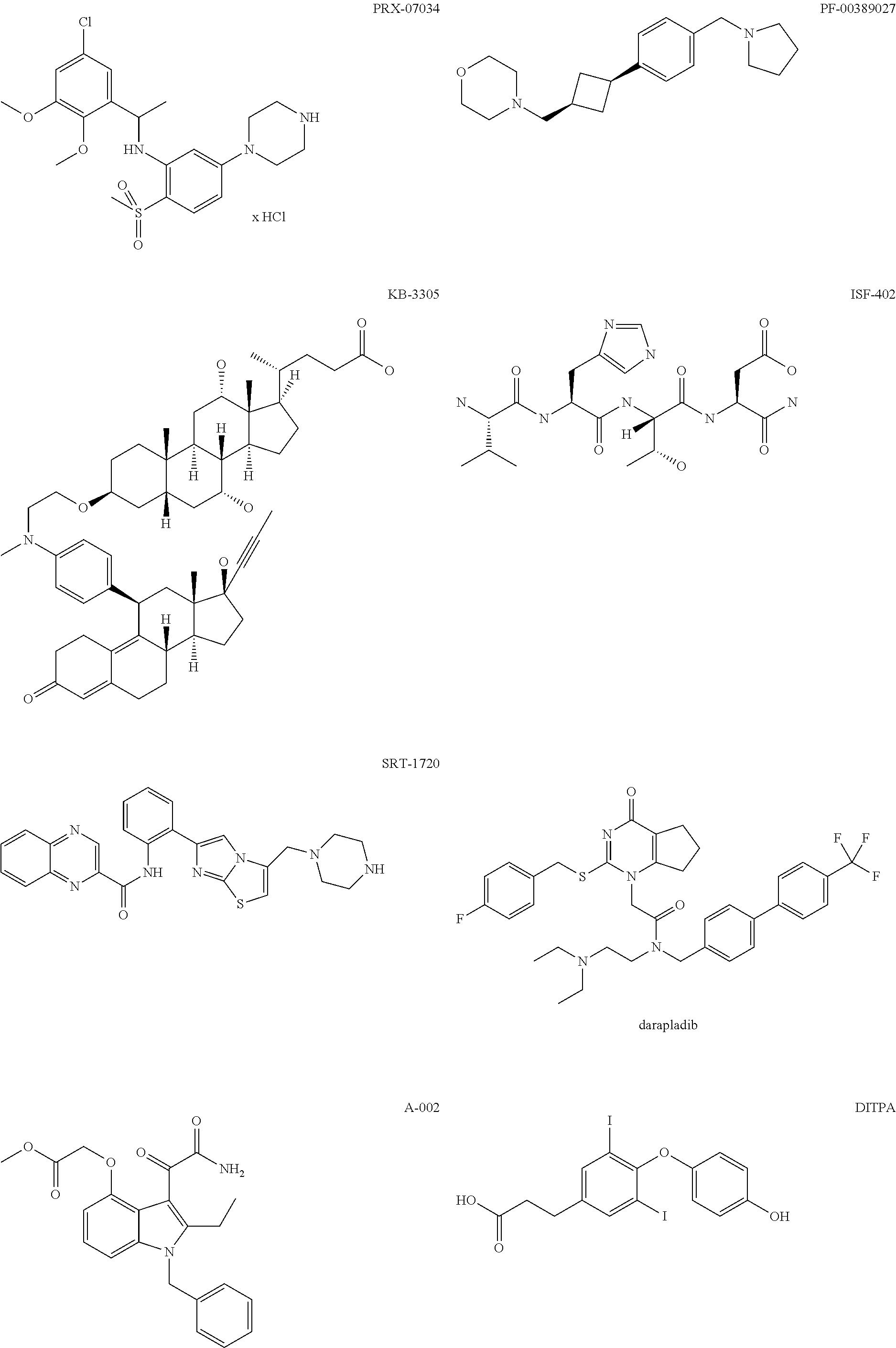

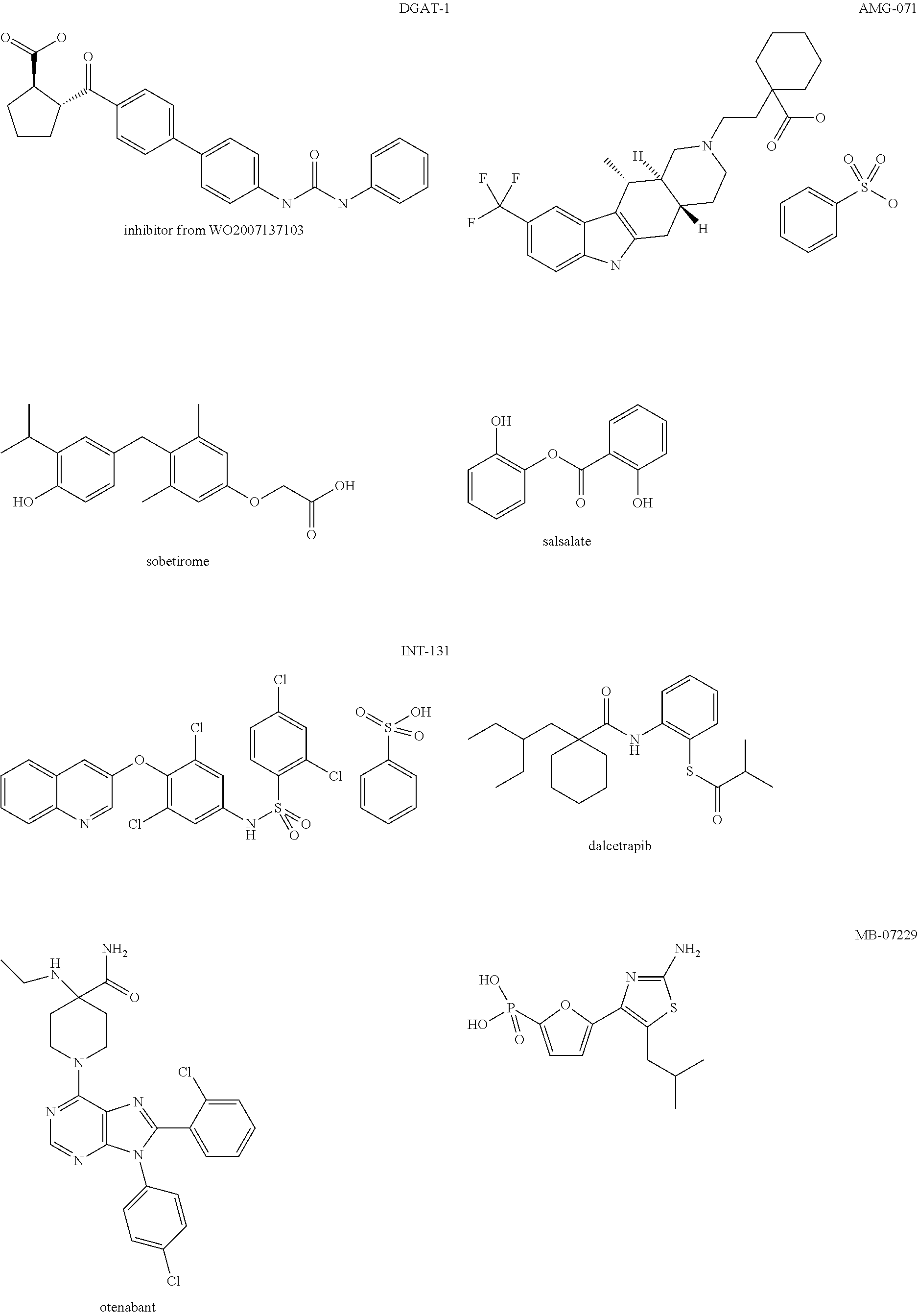

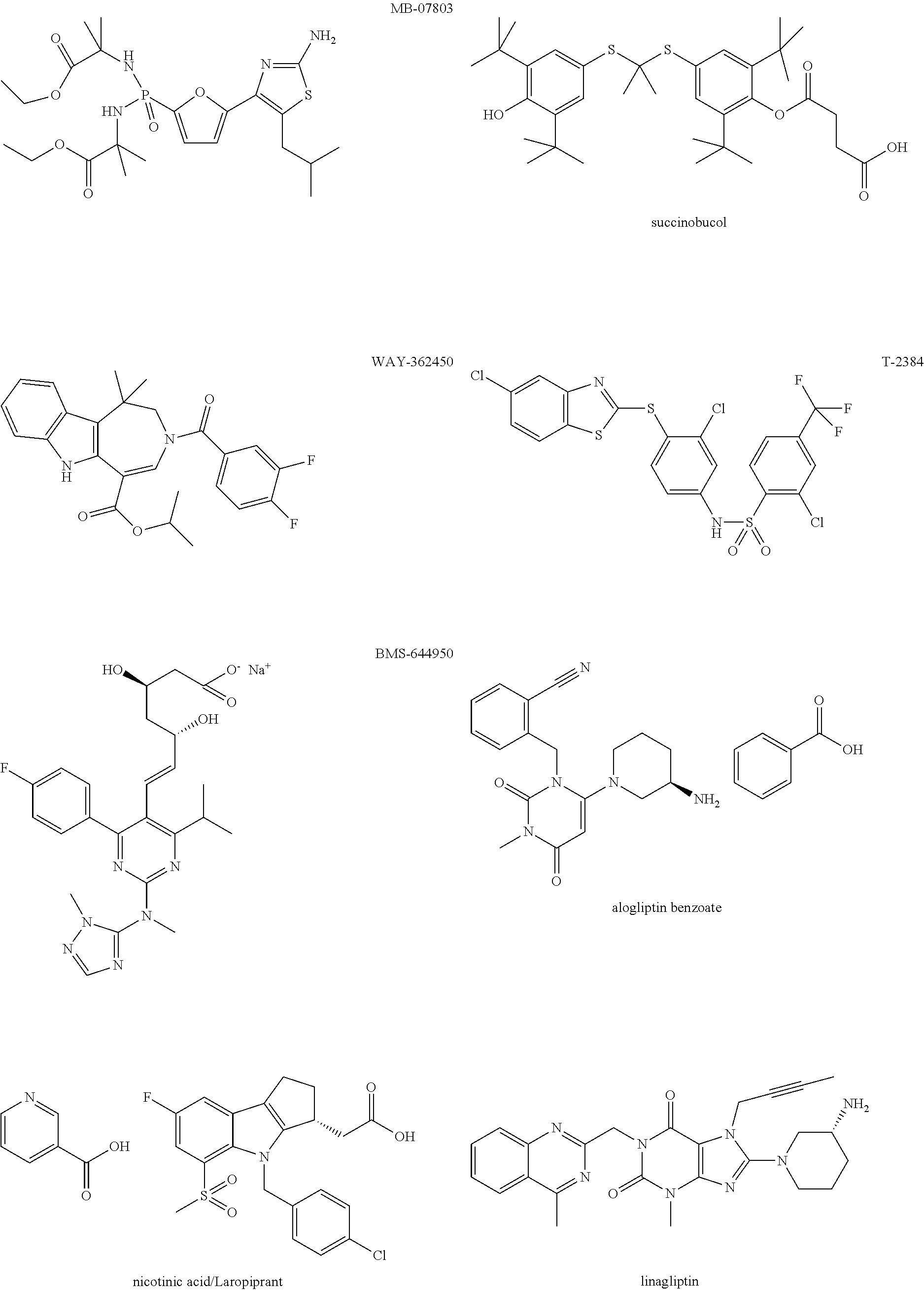

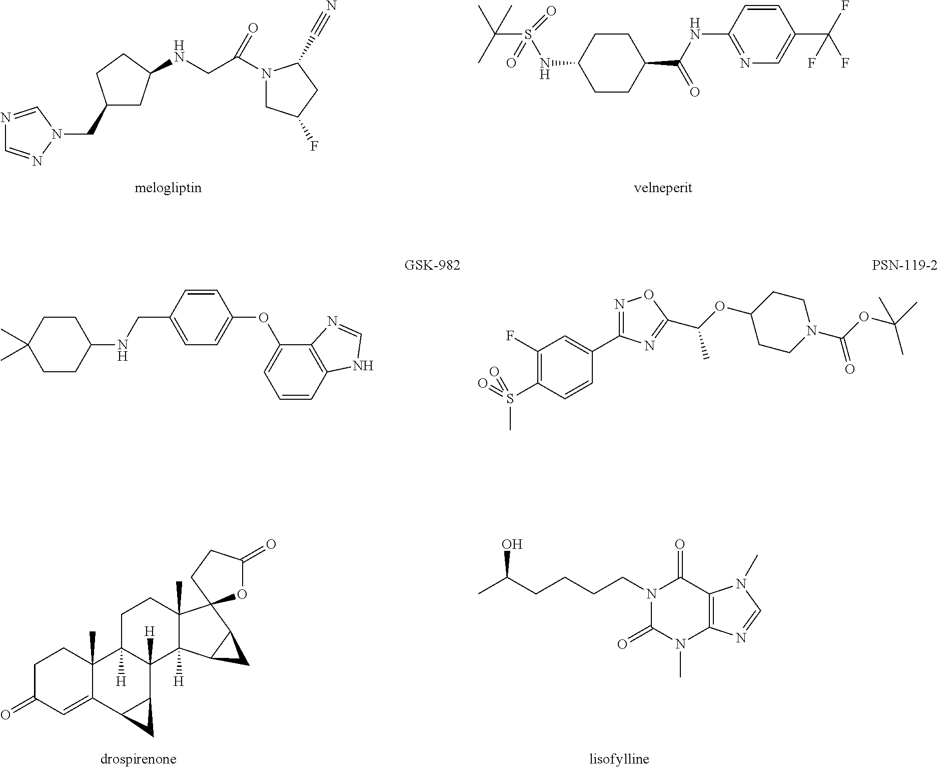

Also suitable are the following active ingredients for combination preparations:

all antiepileptics specified in the Rote Liste 2007, chapter 15;
all antihypertensives specified in the Rote Liste 2007, chapter 17;
all hypotonics specified in the Rote Liste 2007, chapter 19;
all anticoagulants specified in the Rote Liste 2007, chapter 20;
all arteriosclerosis drugs specified in the Rote Liste 2007, chapter 25;
all beta receptors, calcium channel blockers and inhibitors of the renin angiotensin system specified in the Rote Liste 2007, chapter 27;
all diuretics and perfusion-promoting drugs specified in the Rote Liste 2007, chapter 36 and 37;
all withdrawal drugs/drugs for the treatment of addictive disorders specified in the Rote Liste 2007, chapter 39;
all coronary drugs and gastrointestinal drugs specified in the Rote Liste 2007, chapter 55 and 60;
all migraine drugs, neuropathy preparations and Parkinson's drugs specified in the Rote Liste 2007, chapter 61, 66 and 70.

The efficacy of the compounds was tested as follows:

### Inhibition of the Transport Activity of the Human Sodium-Dependent Glucose Transporter 2 (SGLT2, SLC5A2) In Vitro

### 1. Cloning of an Expression Vector for Human SGLT2

The cDNA for human SGLT2 was introduced into the pcDNA4/TO vector (Invitrogen) by means of standard methods of molecular biology as described in Sambrook et al. (Molecular Cloning, A Laboratory Manual, Second Edition). The subsequent sequencing of the insert showed complete identity with bases 21 to 2039 of the base sequence for human SGLT2 which was described by Wells et al. and is deposited in the GenBank sequence database (GenBank Accession Number: M95549). Bases 21 to 2039 correspond to the complete coding region of human SGLT2.

2. Production of a Recombinant Cell Line with Inducible Expression of Human SGLT2

The expression vector for human SGLT2 was introduced into CHO-TREx cells (Invitrogen) by means of FuGene6 lipofection (Roche). To select single cell clones, 600 μg/ml of Zeocin (Invitrogen) was added to the cell culture medium (nutrient mixture F-12 (Ham), (Invitrogen) supplemented with 10% fetal calf serum (FBS Gold, PAA), 10 μg/ml Blasticidin S(CN Biosciences), 100 units/ml penicillin, 100 units/ml streptomycin). The functionality of the single cell clones resulting from the selection was tested via their uptake activity for radiolabeled methyl-α-D-glucopyranoside. That cell clone with the highest uptake activity for methyl-α-D-glucopyranoside, referred to hereinafter as CHO-TRex-hSGLT2, was selected for the further experiments and cultured further in the presence of 600 μg/ml Zeocin.

3. Measurement of the Inhibiting Action of Test Substances on the Uptake of methyl-α-D-glucopyranoside (α-MDG)

CHO-TRex-hSGLT2 cells were seeded in cell culture medium in a concentration of 50 000 cells per well in Cytostar-T scintillating 96-well plates (Amersham Biosciences) and cultivated for 24 h. The expression of the recombinant human SGLT2 was induced by adding 1 μg/ml tetrazykline for a further 24 h. For α-MDG uptake experiments, the cells were washed with PBS and then starved at 37° C. in starvation medium (PBS supplemented with 10% fetal calf serum) for 1 hour. After a further washing step with transport assay buffer (140 mM sodium chloride, 2 mm potassium chloride, 1 mm magnesium chloride, 1 mm calcium chloride, 10 mm HEPES/Tris, pH 7.5), the cells were incubated at room temperature either in the absence or presence of test substances of different concentration for 15 min. The test substances were diluted correspondingly in transport assay buffer proceeding from a 10 mm stock solution in dimethyl sulfoxide (40 μl/well). The assay was subsequently started by adding 10 μl/well of a mixture of radiolabeled methyl-α-D-[U-¹⁴C]glucopyranoside (Amersham) and unlabeled methyl-α-D-glucopyranoside (Acros). The final concentration of methyl-α-D-glucopyranoside in the assay was 50 μM. After an incubation time of 120 min at 37° C., the reaction was stopped by adding 50 μl/well of 10 mM methyl-α-D-glucopyranoside in transport assay buffer (4° C.), and the radioactivity taken up into the cells was determined in a MicroBeta Scintillation Microplate Reader (Wallac).

The half-maximum inhibiting action of the test substances (IC50 value) was determined as follows:
1. Determination of the value for 0% inhibition. This is the measurement in the absence of substance, measured in sodium-containing transport assay buffer.
2. Determination of the value for 100% inhibition. This is the measurement in the absence of substance, measured in sodium-free transport assay buffer (140 mM choline chloride, 2 mM potassium chloride, 1 mM magnesium chloride, 1 mM calcium chloride, 10 mM HEPES/Tris, pH7.5).
3. Calculation of the percentage inhibition values of those measurements which were carried out in the presence of different concentrations of test substance. From this, it was then possible to determine that concentration of the test substance which reduces the uptake of the methyl-α-D-glucopyranoside by 50% (IC50 value).

### Literature:

Wells et al. (1992) Am. J. Physiol. Vol. 263: F459-F465

### Inhibition of the Transport Activity of the Human Sodium-Dependent Glucose Transporter 1 (SGLT1, SLC5A1) In Vitro:

### 1. Cloning of an Expression Vector for Human SGLT1

The cDNA for human SGLT1 was introduced into the pcDNA4/TO vector (Invitrogen) by means of standard methods of molecular biology as described in Sambrook et al. (Molecular Cloning, A Laboratory Manual, Second Edition). The subsequent sequencing of the insert showed complete identity with bases 11 to 2005 of the base sequence for human SGLT1 which was described by Hediger et al. (Hediger et al., Proc. Natl. Acad. Sci. USA 1989, 86, 5748-5752) and is deposited in the GenBank sequence database (GenBank Accession Number: M24847). Bases 11 to 2005 correspond to the complete coding region of human SGLT1.

2. Production of a Recombinant Cell Line with Inducible Expression of Human SGLT1

The expression vector for human SGLT1 was introduced into CHO-TRex cells (Invitrogen) by means of FuGene6 lipofection (Roche). To select single cell clones, 600 μg/ml of Zeocin (Invitrogen) was added to the cell culture medium (nutrient mixture F-12 (Ham), Invitrogen) supplemented with 10% fetal calf serum (BD Biosciences), 10 μg/ml Blasticidin S(CN Biosciences), 100 units/ml penicillin, 100 units/ml streptomycin). The functionality of the single cell clones resulting from the selection was tested via their uptake activity for radiolabeled methyl-α-D-glucopyranoside. That cell clone with the highest uptake activity for methyl-α-D-glucopyranoside, referred to hereinafter as CHO-TRex-hSGLT1, was selected for the further experiments and cultured further in the presence of 600 μg/ml Zeocin.

3. Measurement of the Inhibiting Action of Test Substances on the Uptake of methyl-α-D-glucopyranoside (α-MDG)

CHO-TRex-hSGLT1 cells were seeded in cell culture medium in a concentration of 50 000 cells per well in Cytostar-T scintillating 96-well plates (Amersham Biosciences) and cultivated for 24 h. The expression of the recombinant human SGLT1 was induced by adding 1 μg/ml tetracycline for a further 24 h. For α-MDG uptake experiments, the cells were washed with PBS and then starved at 37° C. in starvation medium (PBS supplemented with 10% fetal calf serum) for 1 hour. After a further washing step with transport assay buffer (140 mM sodium chloride, 2 mM potassium chloride, 1 mM magnesium chloride, 1 mM calcium chloride, 10 mM HEPES/Tris, pH 7.5), the cells were incubated at room temperature either in the absence or presence of test substances of different concentration for 15 min. The test substances were diluted correspondingly in transport assay buffer proceeding from a 10 mM stock solution in dimethyl sulfoxide (40 μl/well). The assay was subsequently started by adding 10 μl of a mixture of radiolabeled methyl-α-D-[U-¹⁴C]glucopyranoside (Amersham) and unlabeled methyl-α-D-glucopyranoside (Acros). The final concentration of methyl-α-D-glucopyranoside in the assay was 50 μM. After an incubation time of 30 min at room temperature, the reaction was stopped by adding 50 μl/well of 10 mM methyl-α-D-glucopyranoside in transport assay buffer (4° C.), and the radioactivity taken up into the cells was determined in a MicroBeta Scintillation Microplate Reader (Wallac).

The half-maximum inhibiting action of the test substances (IC50 value) was determined as follows:
4. Determination of the value for 0% inhibition. This is the measurement in the absence of substance, measured in sodium-containing transport assay buffer.
5. Determination of the value for 100% inhibition. This is the measurement in the absence of substance, measured in sodium-free transport assay buffer (140 mM choline chloride, 2 mM potassium chloride, 1 mM magnesium chloride, 1 mM calcium chloride, 10 mM HEPES/Tris, pH7.5).
6. Calculation of the percentage inhibition values of those measurements which were carried out in the presence of different concentrations of test substance. From this, it was then possible to determine that concentration of the test substance which reduces the uptake of the methyl-α-D-glucopyranoside by 50% (IC50 value).

In vivo pharmacology: determination of urinary glucose excretion and diabetes-related parameters in rats and mice

### Animals

All animal experiments carried out are in accordance with the German Animal Protection Act, and likewise with the international animal health legislation and regulations.

Female Wistar rats (11 weeks old, weight 160 to 180 g) and female CD1 mice (8 weeks old, weight 22 to 25 g) were purchased from the commercial breeder, Charles River, Sulzfeld, Germany. In order to recover from being transported, the animals were given 1 week of time after their arrival. 2 rats and 8 mice per cage (macrolon type 4) were held under controlled conditions at 23° C. and 12:00 h:12:00 h day/night rhythm (the day beginning at 06:00) with ad libitum access to feed (Ssniff standard lab chow) and water. For the collection of urine, the animals were transferred to metabolism cages for 24 h, with feed and water ad libitum. The collection of urine was started from the administration of medicament (t=0 h) to 6 hours (for early effects) and from 6 to 24 hours (for late effects). Rats were held individually in the metabolism cages, mice in groups of two animals. For each dosage and control group, 4 to 8 animals were used.

### Preparation of the Test Compounds for Administration

Each compound was dissolved in water, containing 5% solutol and 0.5% tylose. 5 ml/kg from the solution were administered orally for rats, and 20 ml/kg for mice.

### Determination of the Dose Dependence

The compounds were administered orally in the doses of 3, 10 and 30 mg/kg. Urine volume (Uᵥₒₗ) and urinary glucose concentration were measured in order to determine the urinary glucose excretion (UGE), which is calculated by the formula: UGE=urinary glucose concentration×Uᵥₒₗ×(180/1000). The dose-response curves for the UGE, expressed as g of glucose/kg/24 h, were calculated by regression analysis. The ID₅₀ (mg/kg) values were calculated from the corresponding regression lines, based on 50% inhibition of maximum renal glucose filtration (RGF) of the untreated healthy animals. The RGF was by the formula RGF=GFR×blood glucose concentration, where GFR (glomerular filtration rate)=Uᵥₒₗ×C_{Crea urine}/C_{Crea serum}.

### Analytical Methods and Chemicals

Blood and glucose from the urine were determined enzymatically with a commercially available test: with a Hitachi 912 f (Gluco-quant®Glucose/HK kit, Roche, Germany). Creatinine in serum and urine was analyzed with Crea plus, Roche Diagnostics GmbH, Mannheim, Germany. Urine electrolyte (Na⁺, K⁺, PO₄²⁻, Cl⁻, Ca²⁺) was determined by flame photometry with an EFOX 5053 photometer (Eppendorf).

Table 1 shows the compounds prepared with the data measured in vitro and in vivo

| | | | | | IC50 | IC50 | UGE |
|---|---|---|---|---|---|---|---|
| | Rb, Rc H | | | | SGLT2 | SGLT1 | [g glucose/ |
| Ex. | Ra | R1, R2 | R3 | Cyc1 | [nM] | [μM] | kg/24 h] |
| 1 | H | F, F | Cl | 4-Et-phenyl | 12 | 8 | 25 |
| 2 | H | F, F | Cl | 4-OEt-phenyl | 18 | 8 | 15 |
| 3 | H | H, F | Cl | 4-Et-phenyl | 8 | 0.97 | 5.5 |
| 4 | H | H, F | H | 4-Et-phenyl | 153 | | |
| 5 | H | H, F | H | 4-OCH₃-phenyl | 5 | 0.174 | 7.6 |
| 6 | H | H, F | Cl | 5-Et-thiophene-2 | 12 | 0.475 | 100 |
| 7 | H | H, F | Cl | 2-benzothiophene | 15 | 1.3 | |
| 8 | COOEt | H, F | F | 4-OCH₃-phenyl | 1400 | 6.6 | 34.3 |
| 9 | H | H, F | F | 4-OCH₃-phenyl | 32 | 3.6 | |
| 10 | COOEt | F, F | F | 4-OCH₃-phenyl | 1400 | 13 | |
| 11 | H | F, F | F | 4-OCH₃-phenyl | 54 | 16 | |
| 12 | COOEt | H, F | OCH₃ | 4-OCH₃-phenyl | 2700 | 1.5 | |
| 13 | H | H, F | OCH₃ | 4-OCH₃-phenyl | 31 | 0.152 | |
| 14 | COOEt | F, F | OCH₃ | 4-OCH₃-phenyl | 1600 | | |
| 15 | H | F, F | OCH₃ | 4-OCH₃-phenyl | 80 | 0.611 | |
| 16 | COOEt | H, F | Cl | 4-OEt-phenyl | 2700 | 25 | 30.2 |
| 17 | H | H, F | Cl | 4-OEt-phenyl | 10 | 0.757 | |
| 18 | COOEt | F, F | Cl | 4-OEt-phenyl | 897 | 42 | |
| 19 | Rb COOEt | F, F | Cl | 4-OEt-phenyl | 165 | | 10 |
| 20 | COOEt | H, F | Cl | 4-OCH₃-phenyl | 1060 | | 6.2 |
| 21 | COOEt | F, F | Cl | 4-OCH₃-phenyl | 339 | 50 | 14.1 |
| 22 | H | F, F | Cl | 4-OCH₃-phenyl | 9 | 1.4 | 12 |
| 23 | Rb COOEt | F, F | Cl | 4-OCH₃-phenyl | | | 19.9 |
| 24 | COOEt | H, F | Cl | 4-OCF₃-phenyl | 976 | 23 | 28.1 |
| 25 | H | H, F | Cl | 4-OCF₃-phenyl | 41 | 3.5 | |
| 26 | COOEt | F, F | Cl | 4-OCF₃-phenyl | >10 000 | >10 | |
| 27 | H | F, F | Cl | 4-OCF₃-phenyl | 199 | 16 | |
| 28 | COOEt | F, F | Cl | 5-OCH₃-pyridine-3 | | | 124.8 |
| 29 | H | F, F | Cl | 5-OCH₃-pyridine-3 | 295 | 3.7 | |
| 30 | H | F, F | Cl | 4-OH-phenyl | 15 | 1.2 | |
| 31 | H | F, F | Cl | 4-(O-i-propyl)-phenyl | 42 | 12 | 18 |
| 32 | H | F, F | Cl | 4-(O-CH₂-cyclopropyl)- | 46 | | 230 |
| | | | | phenyl | | | |
| 33 | H | F, F | CH₃ | 4-OH-phenyl | 38 | 2.6 | |
| 34 | H | F, F | CH₃ | 4-OCH₃-phenyl | 27 | 1.5 | |
| 35 | COOEt | H, F | Cl | 3-OCH₃-pyridine-2 | 12 600 | 4.3 | |
| 36 | H | H, F | Cl | 3-OCH₃-pyridine-2 | 304 | 2.7 | |
| 37 | COOEt | F, F | Cl | 4-OCH₃-pyridine-2 | 11 200 | 23.4 | |
| 38 | H | F, F | Cl | 4-OCH₃-pyridine-2 | 393 | 26.5 | |
| 39 | COOEt | H, F | OCF₃ | 4-OCH₃-phenyl | 19 800 | 22.8 | |
| 40 | H | H, F | OCF₃ | 4-OCH₃-phenyl | 412 | 0.232 | |
| 41 | COOEt | F, F | OCF₃ | 4-OCH₃-phenyl | 12 800 | 11.8 | |
| 42 | H | F, F | OCF₃ | 4-OCH₃-phenyl | 982 | 3.3 | |
| 43 | COOEt | H, F | Cl | 4-Cl-phenyl | 3500 | | |
| 44 | H | H, F | Cl | 4-Cl-phenyl | 24 | 0.331 | |
| 45 | COOEt | F, F | Cl | 4-Cl-phenyl | 3000 | | |
| 46 | H | F, F | Cl | 4-Cl-phenyl | 75 | 2.6 | |
| 47 | COOEt | H, F | CF₃ | 4-OCH₃-phenyl | | | |
| 48 | H | H, F | CF₃ | 4-OCH₃-phenyl | 74 | 0.240 | |
| 49 | COOEt | F, F | CF₃ | 4-OCH₃-phenyl | 8000 | >100 | |
| 50 | H | F, F | CF₃ | 4-OCH₃-phenyl | 154 | 7.3 | |
| 51 | COOEt | H, F | CF₃ | 4-Et-phenyl | | | |
| 52 | H | H, F | CF₃ | 4-Et-phenyl | 107 | | |
| 53 | COOEt | F, F | CF₃ | 4-Et-phenyl | | | |
| 54 | H | F, F | CF₃ | 4-Et-phenyl | 257 | | |
| 55 | COOEt | H, F | Br | 4-OCH₃-phenyl | | | |
| 56 | H | H, F | Br | 4-OCH₃-phenyl | 9.4 | 0.180 | |
| 57 | COOEt | H, F | CH₃ | 4-OCH₃-phenyl | | | |
| 58 | H | F, F | CH₃ | 4-OCH₃-phenyl | 7.9 | 0.459 | |
| 59 | COOEt | F, F | CH₃ | 4-OCH₃-phenyl | | | |
| 60 | COOEt | H, F | CH₃ | 4-Et-phenyl | | | |
| 61 | H | H, F | CH₃ | 4-Et-phenyl | 10 | 0.461 | |
| 62 | COOEt | F, F | CH₃ | 4-Et-phenyl | | | |
| 63 | H | F, F | CH₃ | 4-Et-phenyl | | | |
| 64 | H | H, F | cycprop | 4-OCH₃-phenyl | | | |
| 65 | H | H, F | cycprop | 4-Et-phenyl | | | |
| 66 | H | H, F | CH₂- | 4-OCH₃-phenyl | | | |
| | | | cycprop | | | | |
| 67 | H | H, F | CH₂- | 4-Et-phenyl | | | |
| | | | cycprop | | | | |

The examples adduced below serve to illustrate the invention, but without restricting it.

The invention further provides processes for preparing the compounds of the general formula I. The compounds of the general formula I can also be prepared by chemical methods known per se, as described above in the prior art.

The preparation of the examples is described in detail hereinafter.

### EXPERIMENTAL SECTION

### Example 1

### Compound 7

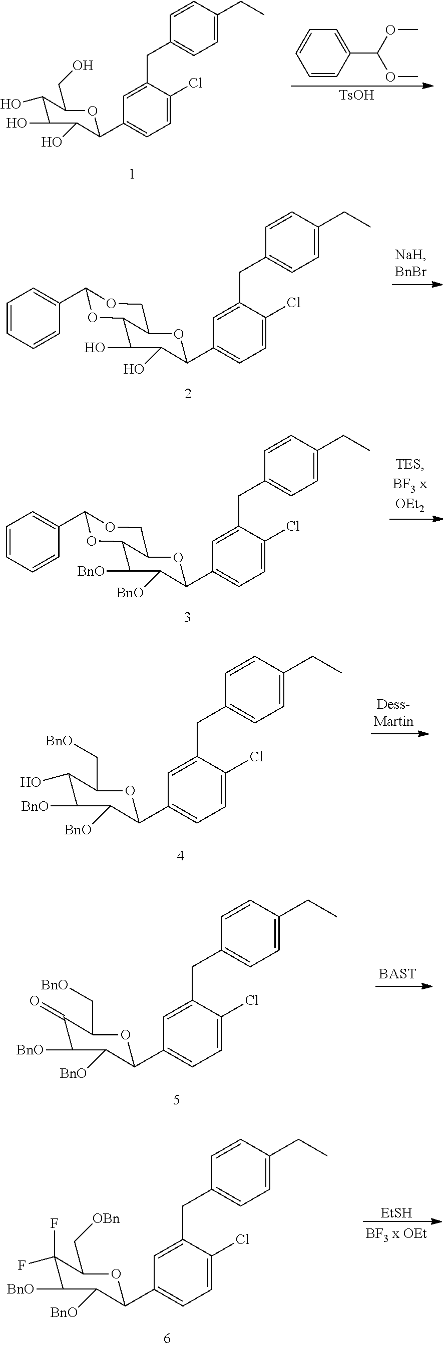

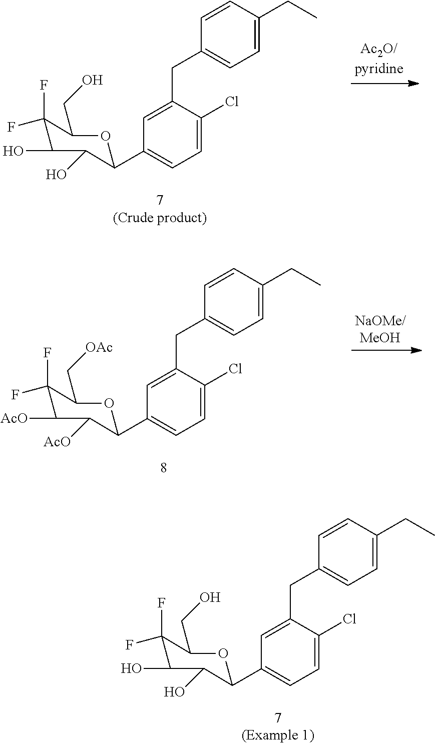

### Synthesis of Compound 2

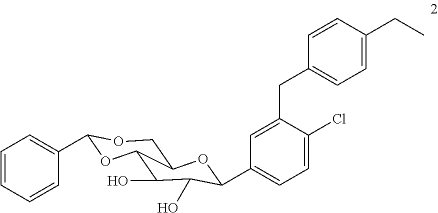

3.2 g (8.1 mmol) of C-glycoside 1 (BMS Patent US 2003/0114390 A1) are dissolved in 80 ml of dimethylformamide and 30 ml of dimethoxybenzaldehyde. After adding 1.5 g of TsOH, the reaction solution is left to stand at room temperature for 20 hours. The reaction solution is then poured onto a mixture of 100 ml of water and 150 ml of ethyl acetate. The organic phase is washed twice more with aqueous NaCl solution, filtered through a little silica gel and concentrated. The residue is separated by chromatography on silica gel (ethyl acetate/n-heptane=1/2 to 2/1). This affords 2.75 g (70% yield) of compound 2 as a colorless solid. C₂₈H₂₉ClO₅ (480.99), MS (ESI⁺) 481.30 (M+H⁺).

### Synthesis of Compound 3

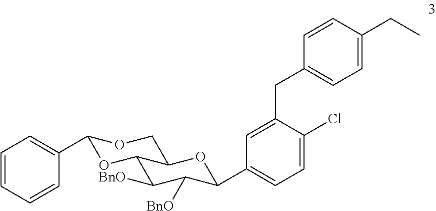

3.2 g (8.1 mmol) of benzylidene derivative 2 are dissolved in 50 ml of dimethylformamide and 4 ml of benzyl bromide. After adding 3 g of sodium hydride (55%) in paraffin oil, the reaction solution is stirred at room temperature for 2 hours. Excess reagents are cautiously destroyed with methanol. The reaction solution is then poured onto a mixture of 100 ml of water and 150 ml of ethyl acetate. The organic phase is washed twice more with aqueous NaCl solution, filtered through a little silica gel and concentrated. The residue is separated by chromatography on silica gel (ethyl acetate/n-heptane=1/6 to 1/1). This affords 2.5 g (59% yield) of compound 3 as colorless crystals from ethyl acetate/n-heptane. C₄₂H₄₁ClO₅ (661.25), MS (ESI⁺) 661.44 (M+H⁺).

### Synthesis of Compound 4

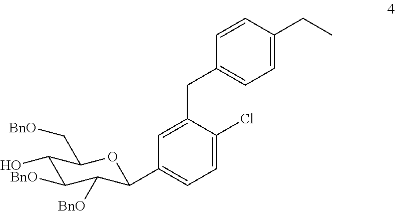

2.9 g (4.4 mmol) of compound 3 are dissolved in 45 ml of methylene chloride and 14 ml of triethylsilane. After addition of 7 ml of boron trifluoride etherate, the reaction solution is left to stand at room temperature for 1 hour. The reaction solution is then poured onto a mixture of 100 ml of water and 150 ml of ethyl acetate. The organic phase is washed once more with aqueous NaCl solution, filtered through a little silica gel and concentrated. The residue is separated by chromatography on silica gel (ethyl acetate/n-heptane=1/4 to 1/1). This affords 1.75 g (60% yield) of compound 4 as a colorless solid. C₄₂H₄₃ClO₅ (663.26), MS (ESI⁺) 680.48 (M+

### Synthesis of Compound 5

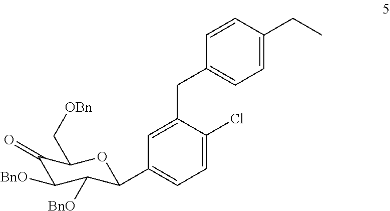

1.75 g (4.4 mmol) of compound 4 are dissolved in 25 ml of 15% Dess-Martin/methylene chloride solution (Aldrich). After 3 hours at room temperature, the reaction solution is poured onto a mixture of 50 ml of saturated aqueous sodium hydrogencarbonate solution and 50 ml of ethyl acetate. The organic phase is washed once more with thiosulfate solution and once with aqueous NaCl solution, filtered through a little silica gel and concentrated. The residue is separated by chromatography on silica gel (ethyl acetate/n-heptane=1/4 to 1/1). This affords 1.56 g (90% yield) of compound 5 as a colorless solid. C₄₂H₄₁=₅ (661.25), MS (ESI⁺) 678.27 (M+NH₄⁺).

### Synthesis of Compound 6

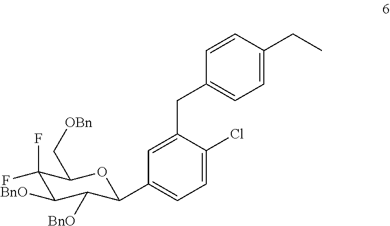

1.56 g (2.4 mmol) of compound 5 are dissolved in 20 ml of methylene chloride and 2 ml of BAST. After 20 hours at room temperature, the reaction solution is poured onto a mixture of 50 ml of saturated aqueous sodium hydrogencarbonate solution and 50 ml of ethyl acetate. The organic phase is washed once more with aqueous NaCl solution, filtered through a little silica gel and concentrated. The residue is separated by chromatography on silica gel (ethyl acetate/n-heptane=1/4 to 1/1). This affords 1.49 g (93% yield) of compound 6 as a colorless solid. C₄₂H₄₁ClF₂O₄ (683.24), MS (ESI⁺) 700.45 (M+NH₄⁺).

### Synthesis of Compound 8

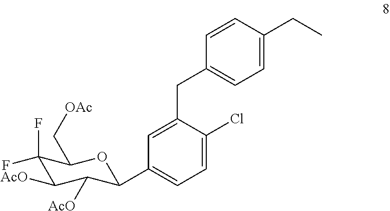

1.49 g (2.2 mmol) of compound 6 are dissolved in 20 ml of thioethanol and 10 ml of boron trifluoride etherate. After 5 hours at room temperature, the reaction solution is poured cautiously onto a mixture of 50 ml of saturated aqueous sodium hydrogencarbonate solution, 50 ml of 10% thiosulfate solution and 50 ml of ethyl acetate. The organic phase is extracted once more with 50 ml of ethyl acetate and the combined organic phase is concentrated. For peracylation, the resulting crude product is admixed with 20 ml of pyridine and 20 ml of acetic anhydride and kept at 60° C. for 1 hour. The mixture is then concentrated twice with 100 ml of toluene. The residue is separated by chromatography on silica gel (ethyl acetate/n-heptane=1/2 to 1/2). This affords 570 mg (49% yield over 2 stages) of compound 8 as colorless crystals from n-heptane with a little ethyl acetate. C₂₇H₂₉ClF₂O₇ (538.98), MS (ESI⁺) 556.30 (M+NH₄⁺).

### Synthesis of Compound 7

### Example 1

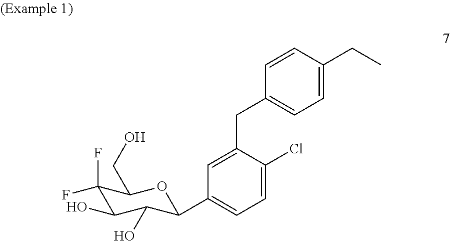

520 mg (0.96 mmol) of peracyl compound 8 are taken up in 3 ml of methylene chloride and 20 ml of methanol and admixed with 1.5 ml of 1 M NaOMe/MeOH. After one hour, the mixture is neutralized with 3 ml of 0.5 M methanolic HCl and concentrated, and the residue is separated by chromatography on silica gel (methylene chloride/methanol/conc. ammonia, 30/5/1). This affords 390 mg (98% yield) of C-glycoside 7 (example 1) as a colorless solid. C₂₁H₂₃ClF₂O₄ (412.86), MS (ESI⁺) 430.24 (M+NH₄⁺).

### Example 2

### Compound 15

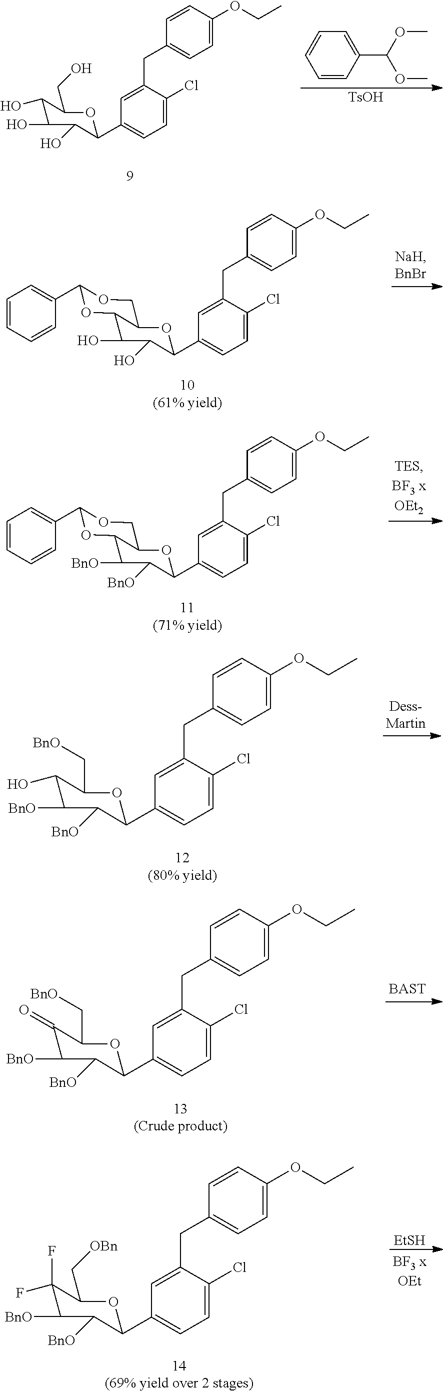

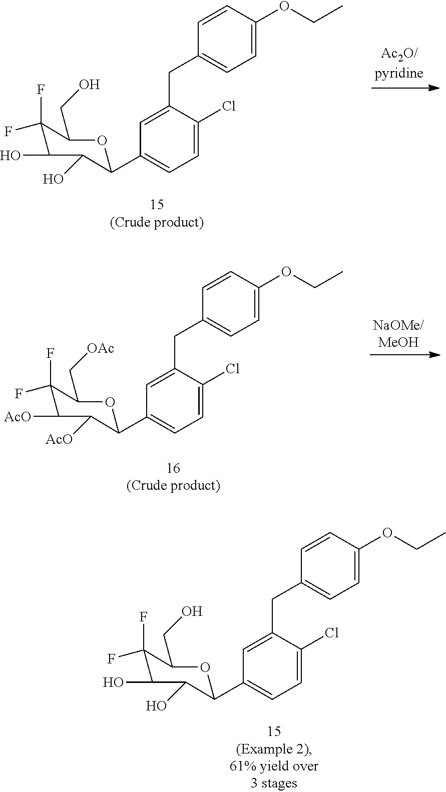

Proceeding from 2.25 g (5.5 mmol) of C-glycoside 9 (BMS Patent US 2003/0114390 A1) via the same reaction sequence as that shown for the preparation of example 1, 320 mg of the difluoro-C-glycoside 15 (example 2) are obtained as a colorless solid. C₂₁H₂₃ClF₂O₅ (428.86), MS (ESI⁺) 446.22 (M+NH₄⁺).

### Example 3

### Compound 28

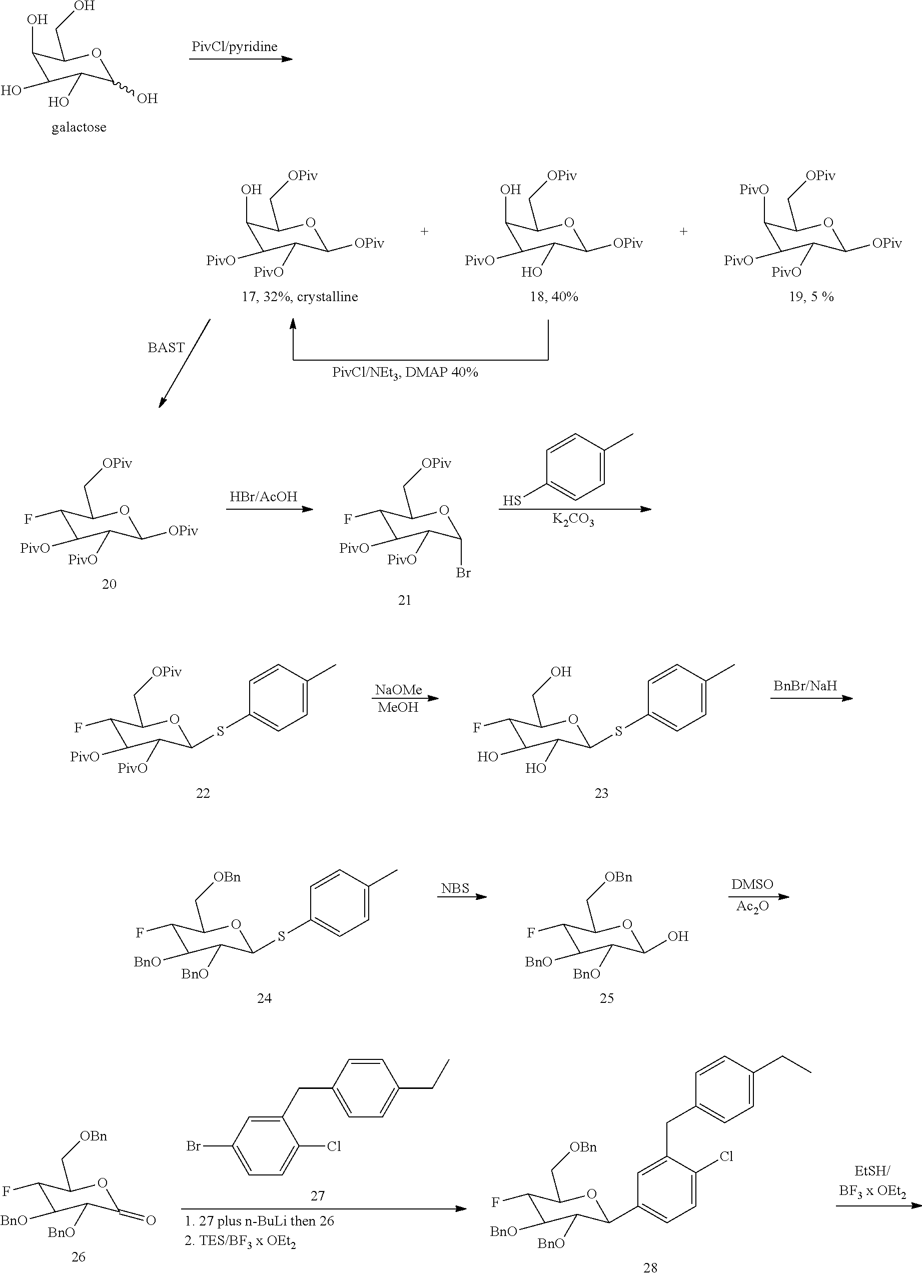

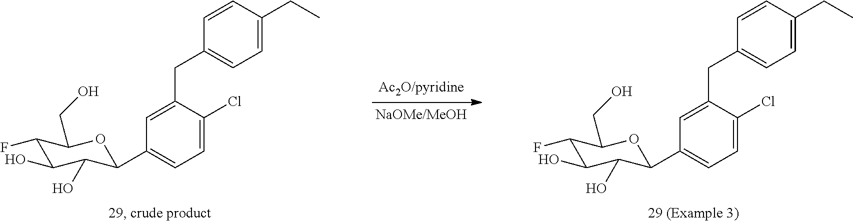

### Synthesis of Compound 17

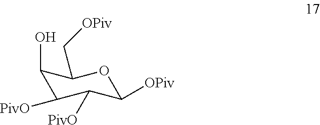

20 g (111 mmol) of galactose are suspended in 160 ml of methylene chloride and 90 ml of pyridine. To this suspension are added first 1 g of DMAP and then, within 20 minutes, 90 ml of pivaloyl chloride. In the course of this, the reaction solution warms up to about 35° C. and an almost clear solution is obtained for a short time, before a suspension (pyridinium chloride precipitate) forms again. To complete the reaction, it is boiled at reflux for another 3 hours. The reaction solution is washed twice with 2N aqueous hydrochloric acid and once with aqueous NaCl solution, filtered through a little silica gel and concentrated. The residue is dissolved in a little ethyl acetate and then diluted with n-heptane until the solubility limit has been attained. This affords 20.3 g (35.4% yield) of crystal fraction 1 and, from the mother liquor after concentration, a further 2.4 g (4.1% yield) of crystal fraction 2. In addition, 54 g of mother liquor are obtained. From this, by reaction with 500 ml of methylene chloride as a solvent and 100 ml of triethylamine, 50 ml of pivaloyl chloride and 5 g of DMAP, it is possible to obtain a further 4.6 g (8.0% yield) of crystalline compound 17 after 20 hours at room temperature (total yield 47.5%). TLC: ethyl acetate/n-heptane 1/3, R_{f}=0.4 for 17, R_{f}=0.3 for 18 and R_{f}=0.5 for 19. C₂₆H₄₄O₁₀ (516.64), MS (ESI⁺) for 17, 534.33 (M+NH₄⁺).

### Synthesis of Compound 20

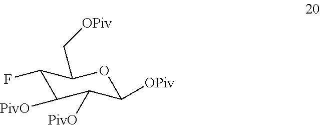

50 g (96.8 mmol) of compound 17 are dissolved in 400 ml of methylene chloride. With water bath cooling, 100 ml of a 50% BAST/THF solution (Aldrich) are added dropwise and the mixture is left to stand at room temperature for 2 hours. The reaction solution is poured cautiously onto ice-water. The organic phase is washed once more with aqueous NaCl solution, filtered through a little silica gel and concentrated. This affords 49 g of compound 20 as a crude product. C₂₆H₄₃FO₉ (518.63), MS (ESI⁺) 536.32 (M+NH₄⁺).

### Synthesis of Compound 21

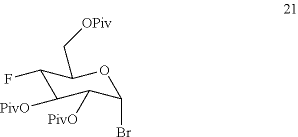

49 g of crude product 20 are dissolved in 250 ml of methylene chloride and 250 ml of 33% HBr in glacial acetic acid and left to stand at room temperature for 1 hour. The reaction solution is then poured onto ice-water and the organic phase is washed twice more with aqueous NaCl solution, filtered through a little silica gel and concentrated. The residue is dissolved in 100 ml and crystallized in an icebath. The precipitate is filtered off with suction and washed with a little cold n-heptane. This affords 20 g of crystalline compound 21 and 34 g of mother liquor (80% content of 21). C₂₁H₃₄BrFO₇ (497.40), MS (ESI⁺) 514.18 (M+NH₄⁺).

### Synthesis of Compound 22

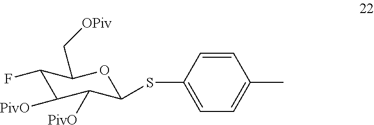

16 g (32.2 mmol) of bromide 21 are dissolved in 50 ml of methylene chloride and 110 ml of dimethylformamide and, after addition of 10 g of 4-methylthiophenol and 20 g of potassium carbonate, stirred at room temperature for 2 hours. The reaction solution is then poured onto water and the organic phase is washed twice more with aqueous NaCl solution, filtered through a little silica gel and concentrated. This affords 25 g of crude product 22.

### Synthesis of Compound 23

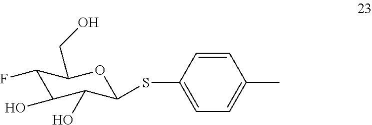

25 g of crude product 22 are taken up in 50 ml of methanol and admixed with 15 ml of 5.6 M NaOMe/MeOH (Fluka). After boiling at reflux for 12 hours, the mixture is neutralized with 2 M methanolic HCl and concentrated, and the residue is suspended with ethyl acetate. The solid is filtered off with suction and the filtrate is concentrated. The resulting residue (18 g) is separated by chromatography on silica gel (ethyl acetate/n-heptane=1/1 to 1/0). This affords 6.7 g (72% yield over 2 stages) of compound 23 as a colorless solid. C₁₃H₁₇FO₄S (288.34), MS (ESI⁺) 306.26 (M+NH₄⁺).

### Synthesis of Compound 24

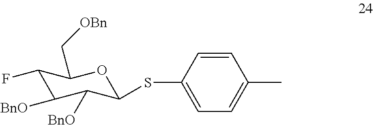

6.1 g (21 mmol) of compound 23 are dissolved in 60 ml of dimethylformamide and 12 ml of benzyl bromide. After adding 5.2 g of sodium hydride (55%) in paraffin oil in portions, the reaction solution is stirred at room temperature for 2 hours. Excess reagents are destroyed cautiously with methanol. The reaction solution is then poured onto a mixture of 100 ml of water and 150 ml of ethyl acetate. The organic phase is washed twice more with aqueous NaCl solution, filtered through a little silica gel and concentrated. The residue is separated by chromatography on silica gel (ethyl acetate/n-heptane=1/6 to 1/2). This affords 9 g (77% yield) of compound 24 as colorless crystals from ethyl acetate/n-heptane. C₃₄H₃₅FO₄S (558.72), MS (ESI⁺) 576.34 (M+NH₄⁺).

### Synthesis of Compound 25

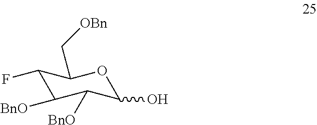

9.0 g (16.1 mmol) of compound 24 are dissolved in 150 ml of technical-grade acetone and admixed with 4.4 g of N-bromosuccinimide. The reaction solution is stirred at room temperature for 2 hours and then the acetone is distilled off on a rotary evaporator. The residue is extracted with a mixture of 100 ml of water and 150 ml of ethyl acetate. The organic phase is washed once more with aqueous NaCl solution, filtered through a little silica gel and concentrated. The residue is separated by chromatography on silica gel (ethyl acetate/n-heptane=1/4 to 2/1). This affords 6.0 g (82% yield) of compound 25 as a colorless solid and 1.2 g of recovered reactant 24. C₂₇H₂₉FO₅ (452.53), MS (ESI⁺) 470.37 (M+NH₄⁺).

### Synthesis of Compound 26

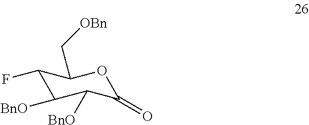

6.0 g (13.3 mmol) of compound 25 are dissolved in 60 ml of dimethyl sulfoxide and 40 ml of acetic anhydride and left to stand at room temperature overnight. The reaction solution is then extracted with a mixture of 100 ml of water and 150 ml of ethyl acetate. The organic phase is washed twice more with aqueous NaCl solution, filtered through a little silica gel and concentrated (evaporate off twice with toluene). The residue is separated by chromatography on silica gel (ethyl acetate/n-heptane=1/4 to 1/2). This affords 5.3 g (89% yield) of compound 26 as a colorless oil, which crystallizes in a freezer cabinet (−25° C.). C₂₇H₂₇FO₅ (450.51), MS (ESI⁺) 451.28 (M+H⁺).

### Synthesis of Compound 28

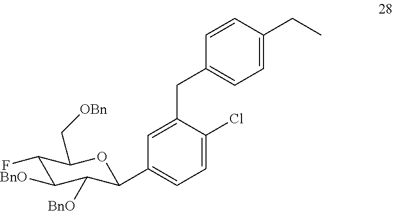

6.0 g (20 mmol) of compound 27 (BMS Patent US 2003/0114390 A1) are dissolved in 100 ml of dry tetrahydrofuran and cooled to −78° C. under argon. 8 ml of a 2.6 M n-BuLi/toluene solution (Aldrich) are added dropwise to this solution. After 10 minutes, a solution of 5.2 g (11.5 mmol) of lactone 26, dissolved in 30 ml of dry tetrahydrofuran, are added dropwise to the reaction solution which is stirred at −78° C. for 30 minutes. The reaction solution is then poured onto a mixture of 100 ml of 10% aqueous ammonium chloride solution and 100 ml of ethyl acetate. The organic phase is washed once more with aqueous NaCl solution, filtered through a little silica gel and concentrated. This affords 11.2 g of crude product, which is dissolved in 150 ml of acetonitrile and 20 ml of triethylsilane, and then cooled to −40° C. under argon. After adding 10 ml of boron trifluoride etherate, the mixture is left to stir at −40° C. for 30 minutes and then the reaction solution is added to a mixture of 100 ml of water and 150 ml of ethyl acetate. The organic phase is washed once more with aqueous NaCl solution, filtered through a little silica gel and concentrated. The residue is separated by chromatography on silica gel (ethyl acetate/n-heptane=1/6 to 1/3). This affords 5 g (65% yield) of compound 28 as a colorless oil. C₄₁H₄₀ClFO₅ (665.25), MS (ESI⁺) 682.39 (M+NH₄⁺).

### Synthesis of Compound 29

### Example 3

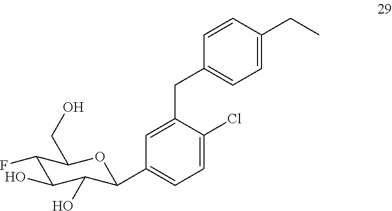

5.0 g (7.5 mmol) of perbenzyl compound 28 are used, via the same deprotection-purification sequence as described for example 1, to obtain 1.47 g (50% yield over 3 stages) of fluorinated C-glycoside 29 (example 3) as a colorless solid. C₂₁H₂₄ClFO₄ (394.87), MS (ESI⁺) 412.24 (M+NH₄⁺).

### Example 4

### Compound 33

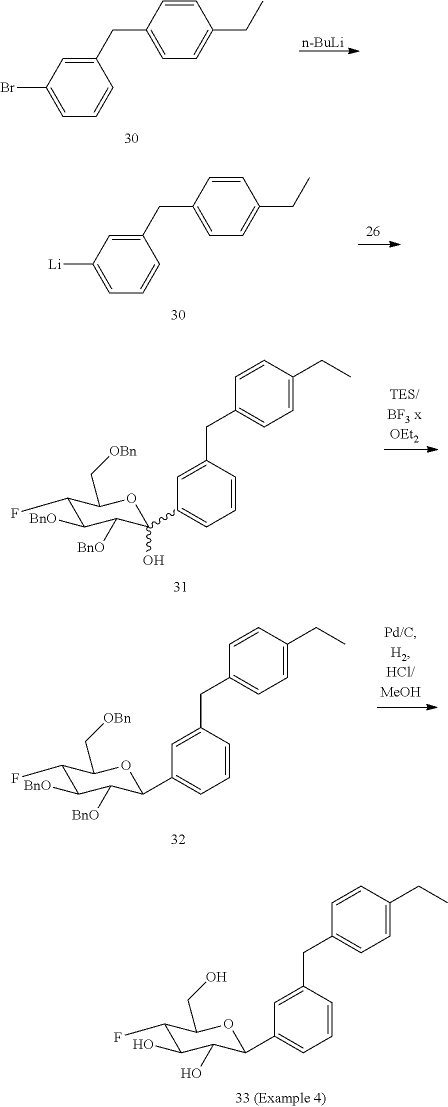

### Synthesis of Compound 31

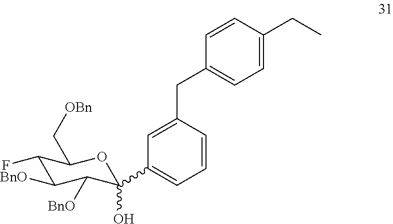

600 mg (2.2 mmol) of compound 30 (BMS Patent US 2003/0114390 A1) are dissolved in 10 ml of dry tetrahydrofuran and cooled to −78° C. under argon. 1.2 ml of a 2.6 M n-BuLi/toluene solution (Aldrich) are added dropwise to this solution. After 10 minutes, a solution of 1.0 g (2.2 mmol) of lactone 26, dissolved in 5 ml of dry tetrahydrofuran, is added dropwise to the reaction solution which is stirred at −78° C. for 30 minutes. The reaction solution is then poured onto a mixture of 20 ml of 10% aqueous ammonium chloride solution and 20 ml of ethyl acetate. The organic phase is washed once more with aqueous NaCl solution, filtered through a little silica gel and concentrated. The residue is separated by chromatography on silica gel (ethyl acetate/n-heptane=1/4 to 1/3). This affords 1.2 g (85% yield) of diastereomer mixture 31 as a colorless oil. C₄₂H₄₃FO₅ (646.81), MS (ESI⁺) 664.14 (M+NH₄⁺).

### Synthesis of Compound 32

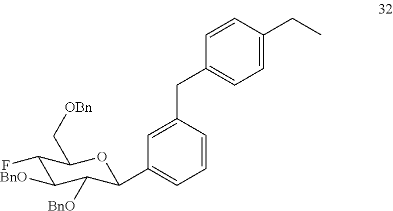

1.2 g (1.85 mmol) of compound 31 are dissolved in 15 ml of acetonitrile and 1.5 ml of triethylsilane, and then cooled to −40° C. under argon. After adding 1.5 ml of boron trifluoride etherate, the mixture is left to stir at −40° C. for 30 minutes and then the reaction solution is added to a mixture of 20 ml of water and 20 ml of ethyl acetate. The organic phase is washed once more with aqueous NaCl solution, filtered through a little silica gel and concentrated. The residue is separated by chromatography on silica gel (ethyl acetate/n-heptane=1/6 to 1/3). This affords 800 mg (68% yield) of compound 32 as a colorless oil. C₄₂H₄₃F04 (630.81), MS (ESI⁺) 648.29 (M+NH₄).

### Synthesis of Compound 33

### Example 4

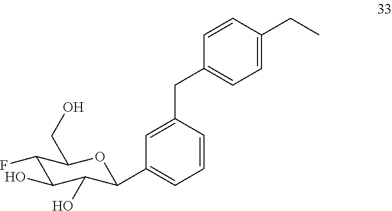

800 mg (1.2 mmol) of compound 32 are dissolved in 30 ml of methylene chloride and 15 ml of 0.5 M HCl/methanol, and then hydrogenated under a hydrogen atmosphere (6 bar) with 200 mg of 10% palladium on activated carbon for 2 hours. The reaction solution is filtered through a little silica gel, washed with methanol and concentrated. The residue is separated by chromatography on silica gel (methylene chloride/methanol/conc. ammonia, 30/5/1). This affords 260 mg (57% yield) of C-glycoside 33 (example 4) as a colorless solid. C₂₁H₂₅FO₄ (360.43), MS (ESI⁺) 378.23 (M+NH₄⁺).

### Example 5

### Compound 37

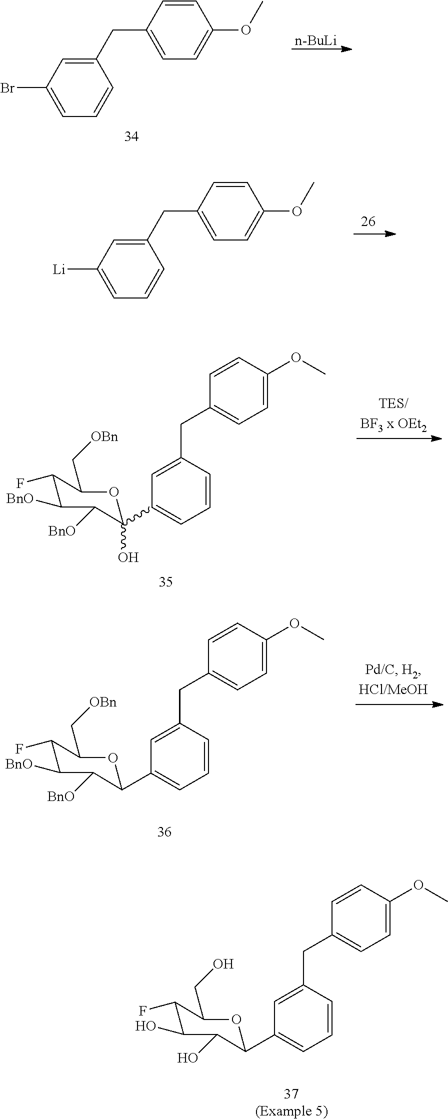

Proceeding from the bromide 34 (BMS Patent US 2003/0114390 A1) and lactone 26, via the same reaction sequence as that shown for the preparation of example 4, the fluoro-C-glycoside 37 (example 5) is obtained as a colorless solid. C₂₀H₂₃FO₅ (362.40), MS (ESI⁺) 380.52 (M+NH₄⁺).

### Example 6

### Compound 42

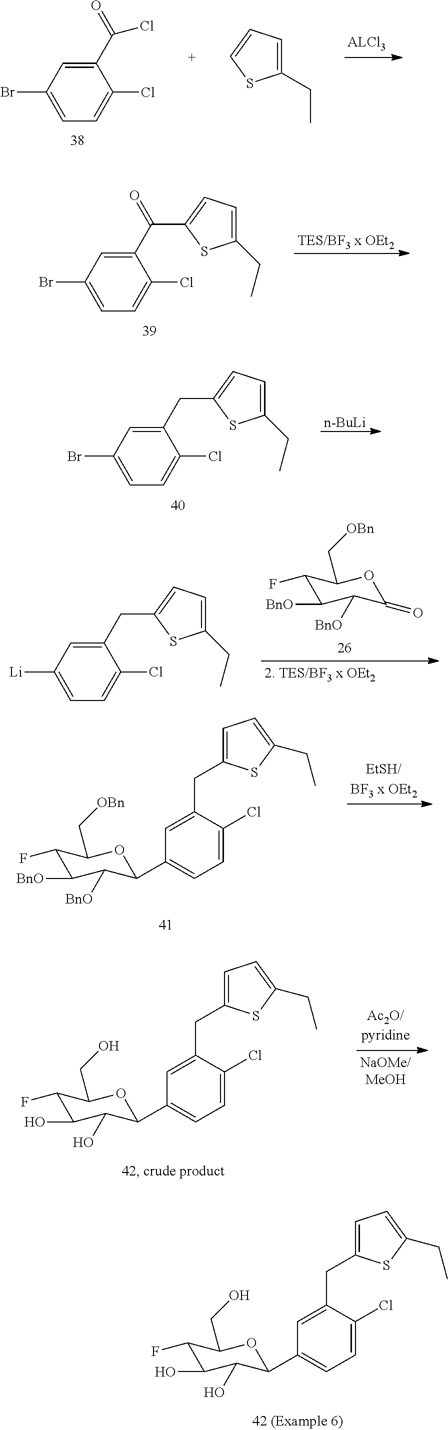

### Synthesis of Compound 39

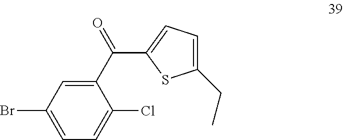

30 g (118 mmol) of compound 38 (BMS Patent WO 2004063209) and 13.4 ml (118 mmol) of 2-ethylthiophene (Aldrich) are dissolved in 50 ml of methylene chloride and cooled to −5° C. under argon. 15.7 g (118 mmol) of aluminum trichloride are added in portions to this solution such that the reaction temperature does not exceed 5° C. The reaction solution is stirred at 5° C. for another 1 hour and then poured onto a mixture of ice cubes and 200 ml of ethyl acetate. The organic phase is washed once more with aqueous NaCl solution, filtered through a little silica gel and concentrated. The residue is separated by chromatography on silica gel (ethyl acetate/n-heptane=0/1 to 1/6). This affords 10.7 g (27% yield) of benzophenone derivative 39 as a colorless oil. C₁₃H₁₀BrClOS (329.65), MS (ESI⁺) 330.22 (M+H⁺).

### Synthesis of Compound 40

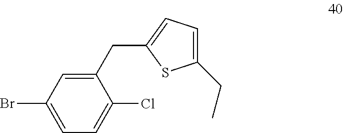

10.7 g (32.5 mmol) of compound 39 are dissolved in 20 ml of acetonitrile, 10 ml of methylene chloride and 12 ml of triethylsilane and then cooled to 10° C. under argon. After adding 6 ml of boron trifluoride etherate, the mixture is allowed to come to room temperature and then left to stand at room temperature overnight. The reaction solution is then added to a mixture of 30 ml of water and 50 ml of ethyl acetate. The organic phase is washed once more with aqueous NaCl solution, filtered through a little silica gel and concentrated. The residue is separated by chromatography on silica gel (ethyl acetate/n-heptane=0/1 to 1/6). This affords 5.3 g (52% yield) of compound 40 as a colorless oil. C₁₃H₁₂BrClS (315.66), MS (ESI⁺) 316.21 (M+H⁺).

### Synthesis of Compound 42

### Example 6

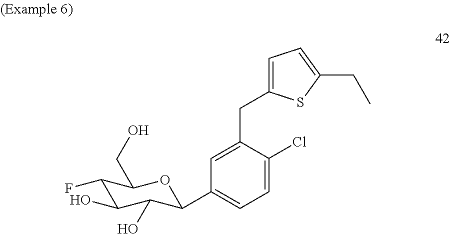

Proceeding from 1.4 g (4.4 mmol) of bromide 40 and lactone 26, via the same reaction sequence as that shown for the preparation of example 4, 220 mg of fluoro-C-thiophene-glycoside 42 (example 6) are obtained as colorless solid. C₁₉H₂₂ClFO₄S (400.90), MS (ESI⁺) 418.22 (M+NH₄⁺).

### Example 7

### Compound 45

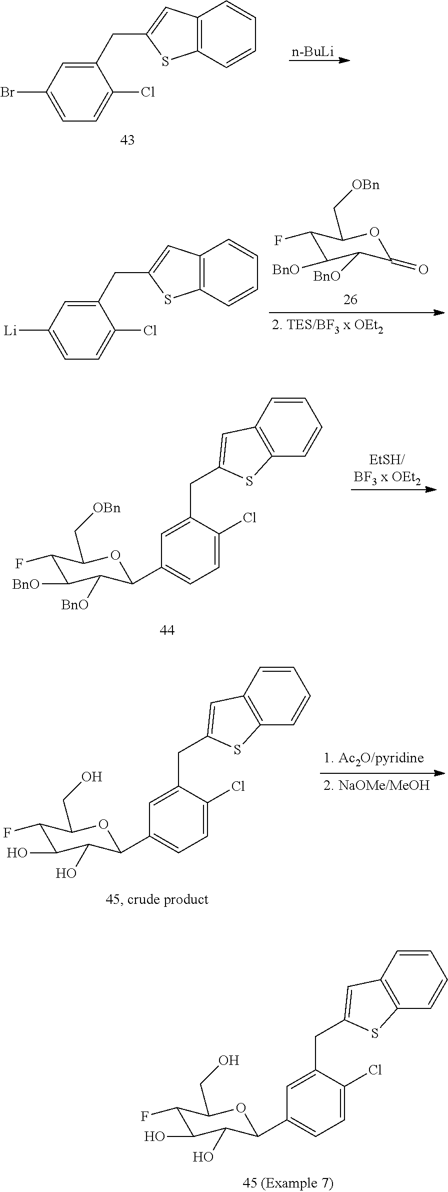

### Synthesis of Compound 45

Proceeding from the bromide 43 (Patent WO 2008013321) and lactone 26, via the same reaction sequence as that shown for the preparation of example 4, the fluoro-C-glycoside 45 (example 7) is obtained as a colorless solid. C₂₁H₂₀ClFO₄S (422.91), MS (ESI⁺) 440.16 (M+NH₄⁺).

### Example 8

### Compound 46

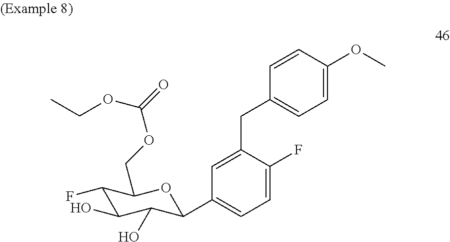

### Synthesis of Compound 47

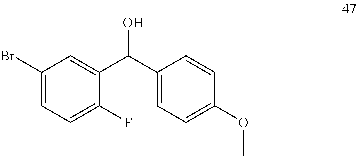

12.9 g (69.0 mmol) of 4-bromoanisole are dissolved in 130 ml of dry tetrahydrofuran (THF) and cooled to −78° C. with an acetone/dry ice mixture under an argon atmosphere. After adding 26.5 ml of a 2.6 molar n-butyllithium solution in toluene (69 mmol), the reaction solution is stirred at −78° C. for 20 minutes. A solution of 10 g (49.3 mmol) of 5-bromo-2-fluorobenzaldehyde in 70 ml of THF is then added dropwise to the reaction solution which is stirred at −78° C. for 1 hour. The solution is poured onto 200 ml of 10% ammonium chloride solution and 200 ml of ethyl acetate. The organic phase is washed once with aqueous NaCl solution, filtered through a little silica gel and concentrated. This affords 20 g of crude product 47 as a colorless oil.

### Synthesis of Compound 48

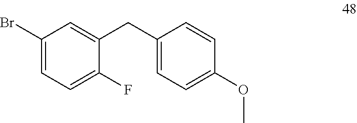

20 g of crude product 47 are dissolved in 200 ml of acetonitrile, 200 ml of methylene chloride and 30 ml of triethylsilane and cooled to −40° C. with an acetone/dry ice mixture under an argon atmosphere. After adding 16 ml of boron trifluoride etherate, the reaction solution is left to stir at −40° C. for 20 minutes. The reaction solution is then poured onto a mixture of 200 ml of saturated sodium chloride solution and 200 ml of ethyl acetate. The organic phase is washed once more with aqueous NaCl solution, filtered through a little silica gel and concentrated. The residue is separated by chromatography on silica gel (ethyl acetate/n-heptane=1/6 to 1/4). This affords 9.3 g (64% yield over 3 stages) of product 48 as a colorless oil.

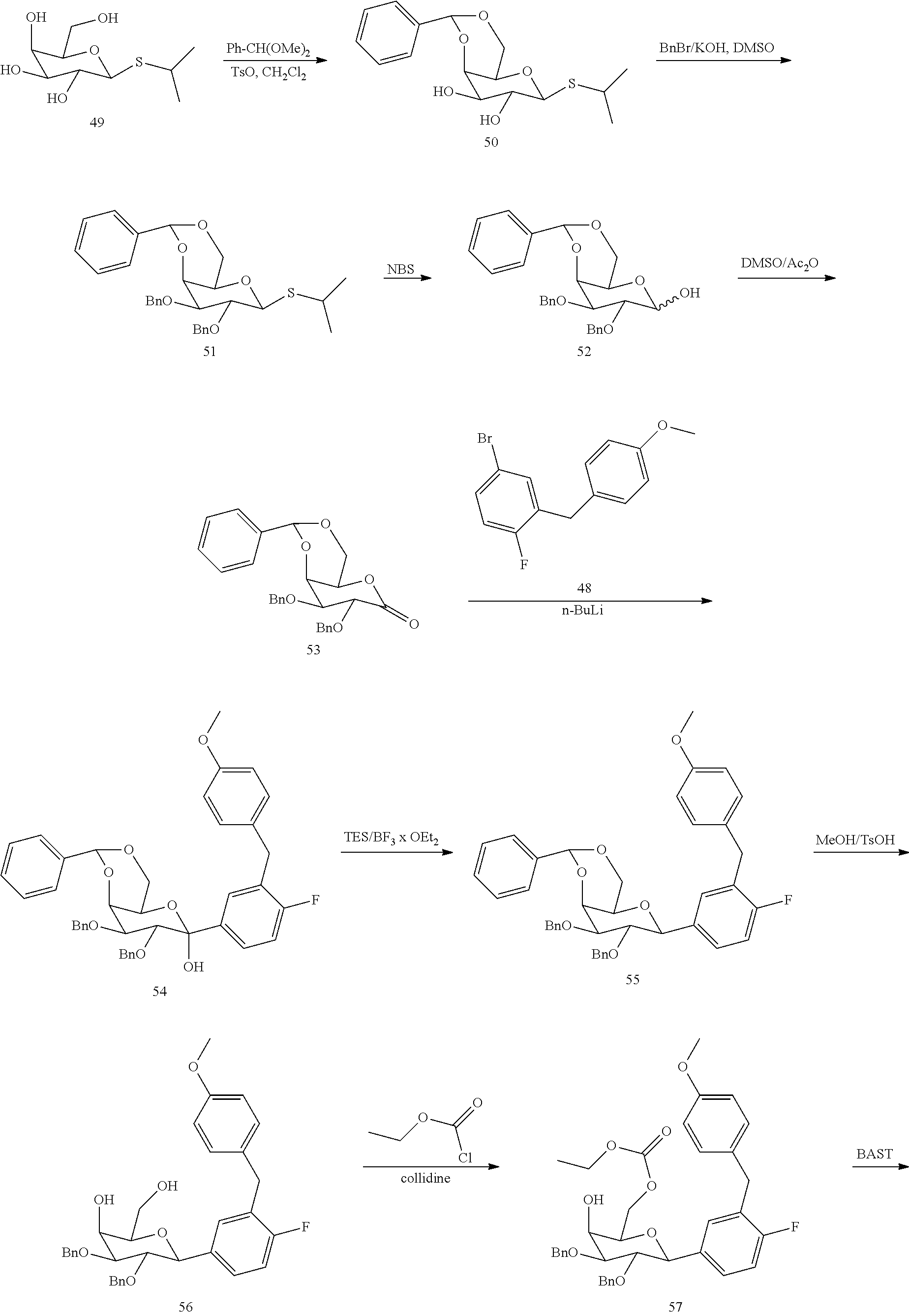

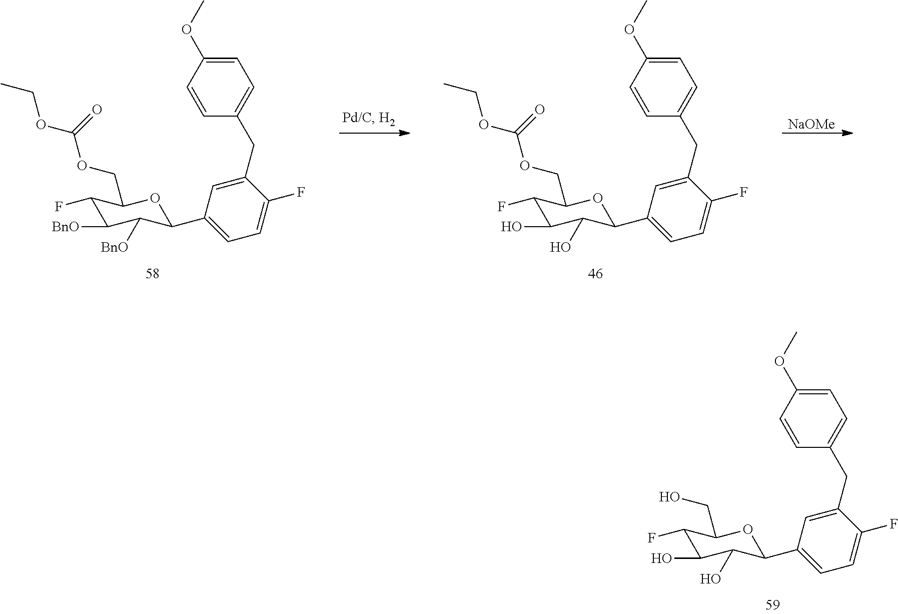

### Synthesis of Compound 50

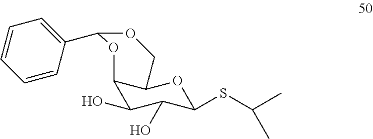

100 g (420 mmol) of isopropyl-beta-D-galactopyranoside 49 are suspended in 1 l of methylene chloride and, after adding 100 ml of benzaldehyde dimethyl acetal and 1 g of para-toluenesulfonic acid, stirred at room temperature for 2 hours. After about 2 hours, the reactant has dissolved to give a clear solution. After adding 5 ml of triethylamine, the organic phase is filtered through about 150 ml of silica gel and washed with 500 ml of ethyl acetate. About 700 ml of solvent are removed on a rotary evaporator. The product crystallizes out of this solution within one hour. The product is filtered off with suction and washed with ethyl acetate/n-heptane=1/3. Further concentration of the mother liquor affords a second crystal fraction with somewhat lower purity. 112 g of crystal fraction 1 and 20 g of crystal fraction 2 (total yield 96%) of benzylidene derivative 50 are obtained.

### Synthesis of Compound 51

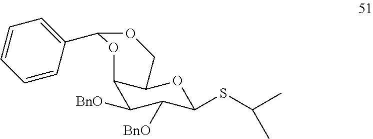

60 g (184 mmol) of galactose derivative 50 are dissolved in 1.2 l of DMSO and 96 ml of benzyl bromide. A total of 72 g of potassium hydroxide powder is added in portions to the mixture, in the course of which the reaction solution is kept between 30 and 40° C. At a reaction temperature below 30° C. and above 40° C., poorer yields are obtained. After adding the complete amount of base, the mixture is left to stir at room temperature for another hour. The solution is poured onto 1 l of water and 1 l of ethyl acetate/n-heptane (1:1). The organic phase is washed twice more with aqueous NaCl solution, filtered through silica gel, washed with ethyl acetate/n-heptane (1:1) and concentrated. This affords 94.3 g of a crystal fraction 51 which is slightly contaminated.

### Synthesis of Compound 52

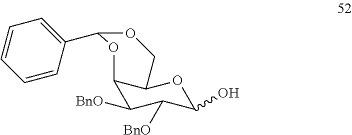

94.3 g of galactose derivative 51 are dissolved in 1.1 l of acetone and 100 ml of water. After adding 31.5 g of N-bromosuccinimide (NBS), the mixture is left to stir at room temperature for 15 minutes. About 600 ml of acetone are distilled off on a rotary evaporator. The remaining solution is poured onto 1 l of water and 1 l of ethyl acetate. The organic phase is washed twice more with aqueous NaCl solution, filtered through silica gel, washed with ethyl acetate/n-heptane (1:1) and concentrated until crystallization commences. This affords 69.2 g of crystalline product 52 (84% yield over 2 stages).

### Synthesis of Compound 53

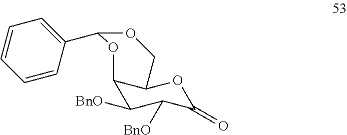

The alcohol 53 is oxidized analogously to the literature method (Helvetica Chimica Acta—Vol. 89 (2006) page 648, compound 17) to the lactone 53 (96% yield).

### Synthesis of Compound 54

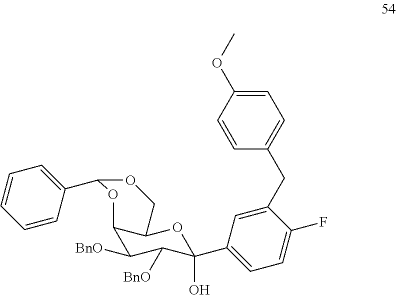

3.3 g (11.2 mmol) of bromide 48 are dissolved in 60 ml of dry tetrahydrofuran (THF) and cooled to −78° C. with an acetone/dry ice mixture under an argon atmosphere. After adding 5 ml of a 2.6 molar n-butyllithium solution in toluene (13 mmol), the reaction solution is stirred at −78° C. for 20 minutes. A solution of 5.0 g (11.2 mmol) of lactone 53 in 15 ml of THF is then added dropwise to the reaction solution which is stirred at −78° C. for one hour. The solution is poured onto 50 ml of 10% ammonium is chloride solution and 50 ml of ethyl acetate. The organic phase is washed once more with aqueous NaCl solution, filtered through a little silica gel and concentrated. 9 g of crude product 54 are obtained as a colorless oil.

### Synthesis of Compound 55

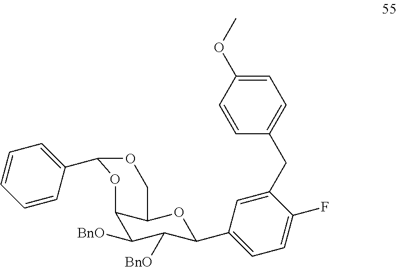

9 g of crude product 54 are dissolved in 60 ml of acetonitrile, 60 ml of methylene chloride and 10 ml of triethylsilane and cooled to −40° C. with an acetone/dry ice mixture under an argon atmosphere. After adding 5 ml of boron trifluoride etherate, the reaction solution is left to stir at −40° C. for 20 minutes. The reaction solution is then poured onto a mixture of 100 ml of saturated sodium chloride solution and 100 ml of ethyl acetate. The organic phase is washed once with aqueous NaCl solution, filtered through a little silica gel and concentrated. The residue is separated by chromatography on silica gel (ethyl acetate/n-heptane=1/3 to 2/1). This affords 2.8 g (39% yield over 3 stages) of product 55 as a colorless solid. C₄₁H₃₉FO₆ (646.76), MS (ESI⁺) 647.30 (M+

### Synthesis of Compound 56

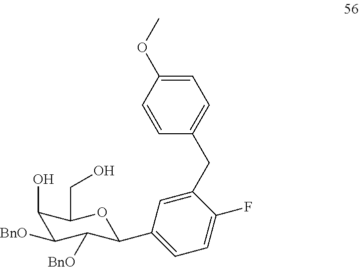

2.8 g of C-glycoside 55 is dissolved in 10 ml of methylene chloride and 30 ml of methanol and, after adding 300 mg of para-toluenesulfonic acid, heated to 50° C. on a rotary evaporator for 1 hour (which distils off the methylene chloride). After adding 1 ml of triethylamine, the solvent is distilled off. The residue is separated by chromatography on silica gel (ethyl acetate/n-heptane=1/1 to 1/0). This affords 2.0 g (83% yield) of product 56 as a colorless solid.

### Synthesis of Compound 57

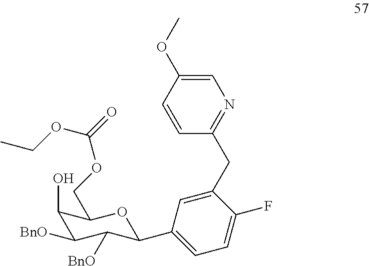

2.03 g of diol 56 are dissolved in 30 ml of collidine and, after adding 2 ml of ethyl chloroformate, stirred at room temperature for 20 hours. The reaction solution is then poured onto a mixture of 50 ml of 2N aqueous HCl solution and 50 ml of ethyl acetate. The organic phase is washed once more with 50 ml of 2N aqueous HCl solution and once with aqueous NaCl solution, filtered through a little silica gel and concentrated. The residue is separated by chromatography on silica gel (ethyl acetate/n-heptane=1/2 to 1/1). This affords 2.0 g (87% yield) of carbonate 57 as a colorless solid. C₃₇H₃₉FO₈ (630.72), MS (ESI⁺) 631.33 (M+H⁺).

### Synthesis of Compound 58

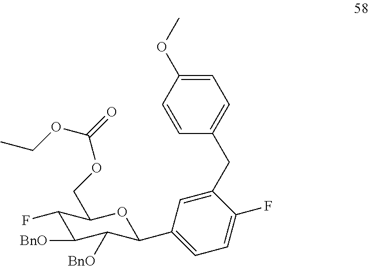

900 mg (1.4 mmol) of galactose derivative 57 are dissolved in 10 ml of methylene chloride. While cooling with a water bath, 2 ml of a 50% BAST/THF solution (Aldrich) are added dropwise and the mixture is left to stand at room temperature for 1 hour. The reaction solution is poured cautiously onto ice-water. The organic phase is washed once more with aqueous NaCl solution, filtered through a little silica gel and concentrated. The residue is separated by chromatography on silica gel (ethyl acetate/n-heptane=1/3 to 1/1). This affords 680 mg (75% yield) of fluoroglucose derivative 58 as a colorless solid. C₃₇H₃₈FO₇ (632.71), MS (ESI⁺) 650.32 (M+NH₄⁺).

### Example 8

### Compound 46

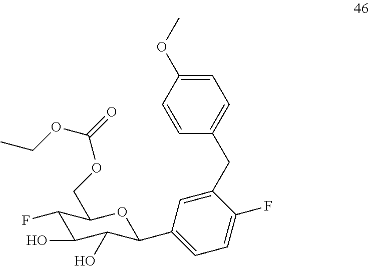

600 mg of compound 58 are dissolved in 20 ml of methylene chloride and, after adding 120 mg of palladium on activated carbon (10% Pd), hydrogenated at 6 bar of hydrogen pressure at room temperature for 2 hours. The reaction solution is then filtered through a little silica gel, washed with ethyl acetate and concentrated. The residue is separated by chromatography on silica gel (ethyl acetate/n-heptane=1/2 to 1/1). This affords 244 mg (57% yield) of carbonate 46 (example 8) as a colorless solid. C₂₃H₂₆FO₇ (452.46), MS (ESI⁺) 453.13 (M+H⁺).

### Example 9

### Compound 59

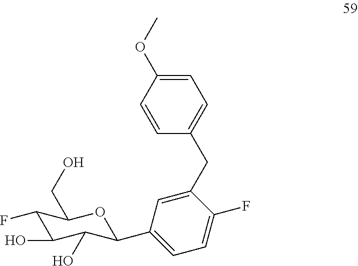

122 mg (0.27 mmol) of compound 46 are taken up in 20 ml of methanol and admixed with 1 ml of 1 M NaOMe/MeOH. After one hour, the mixture is neutralized with 2 ml of 0.5 M methanolic HCl and concentrated, and the residue is separated by chromatography on silica gel (ethyl acetate/n-heptane=1/1 to 1/0). This affords 90 mg (88% yield) of product 59 (example 9) as a colorless solid. C₂₀H₂₂F₂O₅ (380.39), MS (ESI⁺) 403.13 (M+Na⁺).

### Example 10

### Compound 60

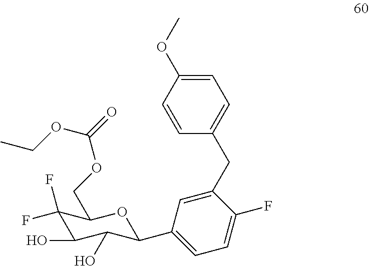

### Synthesis of Compound 61

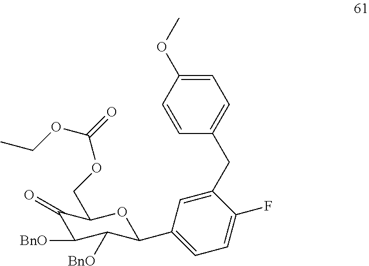

1.0 g (1.6 mmol) of carbonate 57 is dissolved in 18 ml of 15% Dess-Martin/methylene chloride solution (Aldrich). After 38 hours at room temperature, the reaction solution is poured on to a mixture of 50 ml of saturated aqueous sodium hydrogencarbonate solution and 50 ml of ethyl acetate. The organic phase is washed once more with thiosulfate solution and once with aqueous NaCl solution, filtered through a little silica gel and concentrated. This affords 1.2 g of crude product 61 as a colorless oil.

### Synthesis of Compound 62

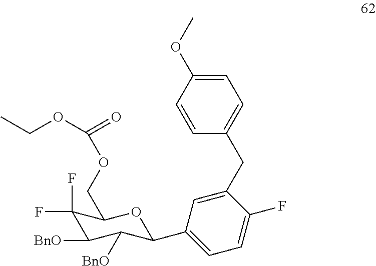

1.2 g of ketone 61 are dissolved in 12 ml of methylene chloride. While cooling with a water bath, 3.6 ml of a 50% BAST/THF solution (Aldrich) are added dropwise and the mixture is left to stand at room temperature for 40 hours. The reaction solution is poured cautiously onto ice-water. The organic phase is washed once more with aqueous NaCl solution, filtered through a little silica gel and concentrated. The residue is separated by chromatography on silica gel (ethyl acetate/n-heptane=1/6 to 1/2). This affords 740 mg (74% yield over 2 stages) of difluoroglucose derivative 62 as a colorless solid. C₃₇H₃₇F₃O₇ (650.70), MS (ESI⁺) 668.29 (M+NH₄).

### Synthesis of Compound 60

### Example 10

740 mg (1.14 mmol) of compound 62 are dissolved in 20 ml of methylene chloride and, after adding 150 mg of palladium on activated carbon (10% Pd), hydrogenated at 6 bar of hydrogen pressure at room temperature for 2 hours. The reaction solution is then filtered through a little silica gel, washed with ethyl acetate and concentrated. The residue is separated by chromatography on silica gel (ethyl acetate/n-heptane=1/2 to 1/1). This affords 275 mg (51% yield) of carbonate 60 (example 10) as a colorless solid.

### Example 11

### Compound 63

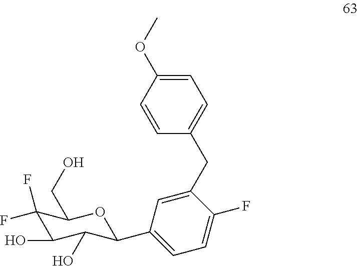

240 mg (0.51 mmol) of compound 60 are taken up in 20 ml of methanol and admixed with 2 ml of 1 M NaOMe/MeOH. After one hour, the mixture is neutralized with 4 ml of 0.5 M methanolic HCl and concentrated, and the residue is separated by chromatography on silica gel (ethyl acetate/n-heptane=1/1 to 1/0). This affords 140 mg (67% yield) of product 63 (example 11) as a colorless solid. C₂₀H₂₁F₃O₅ (398.38), MS (ESI⁺) 416.44 (M+NH₄⁺).

### Example 12 and 13

### Compound 64 and 65

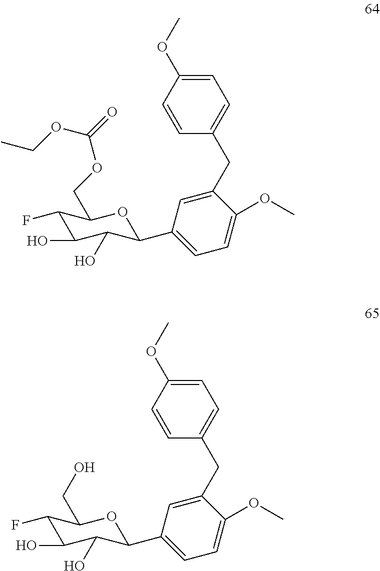

The C-glycosides 64 and 65 are prepared analogously to the method for the synthesis of example 8 and 9, proceeding from 4-bromoanisole and 5-bromo-2-methoxybenzaldehyde, over 9 stages with similar yields.

MS for compound 64: C₂₄H₂₉FO₈ (464.49), MS (ESI⁺) 482.14 (M+NH₄).

MS for compound 65: C₂₁H₂₅FO₆ (392.43), MS (ESI⁺) 410.23 (M+NH₄).

### Example 14 and 15

### Compound 66 and 67

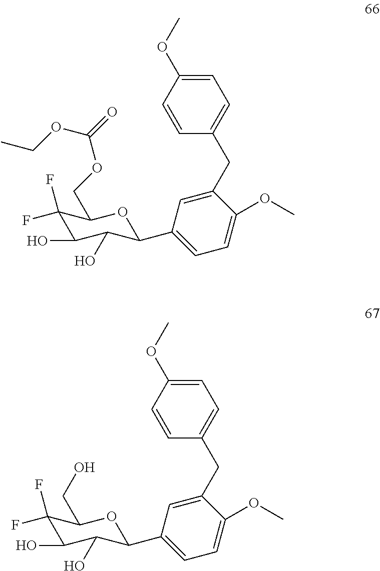

The C-glycosides 66 and 67 are prepared analogously to the method for the synthesis of example 10 and 11, proceeding from 4-bromoanisole and 5-bromo-2-methoxybenzaldehyde, over 10 stages with similar yields.

MS for compound 66: C₂₄H₂₈F₂O₈ (482.48), MS (ESI⁺) 500.20 (M+NH₄⁺).

MS for compound 67: C₂₁H₂₄F₂O₆ (410.42), MS (ESI⁺) 428.22 (M+NH₄).

### Example 16 and 17

### Compound 68 and 69

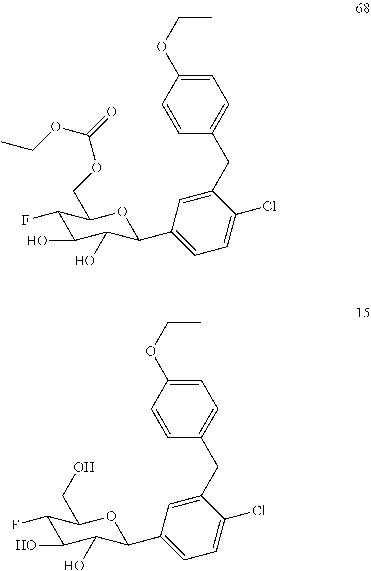

The C-glycosides 68 and 69 are prepared analogously to the method for the synthesis of example 8 and 9, proceeding from 4-bromo-1-chloro-(4-ethoxybenzyl)benzene and lactone 53, with similar yields.

MS for compound 68: C₂₄H₂₈ClFO₇ (482.94), MS (ESI⁺) 482.16 (M−H₂O+NH₄⁺).

MS for compound 69: C₂₁H₂₄ClFO₅ (410.87), MS (ESI⁺) 428.42 (M+NH₄⁺).

### Example 18 and 2

### Compound 70 and 15

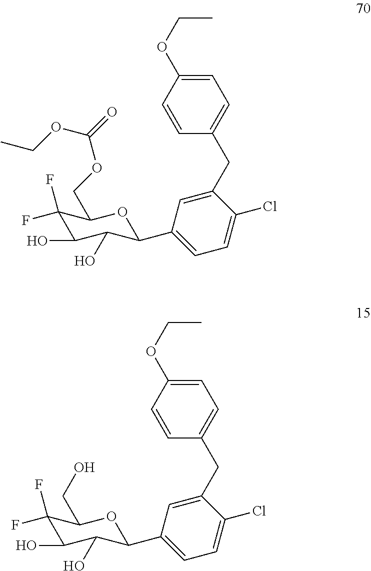

The C-glycosides 68 and 15, which has already been described via another synthesis route, are prepared analogously to the method for the synthesis of example 10 and 11, proceeding from 4-bromo-1-chloro-(4-ethoxybenzyl)benzene and lactone 53, with similar yields.

MS for compound 70: C₂₄H₂₇ClF₂O₇ (500.93), MS (ESI⁺) 483.13 (M−H₂O+H⁺).

### Example 19

### Compound 71

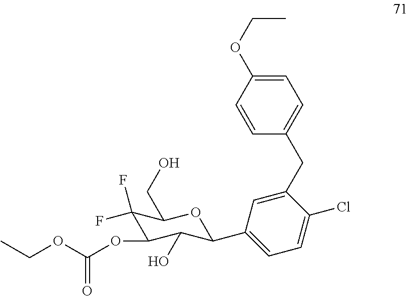

220 mg (0.51 mmol) of compound 15 are dissolved in 4 ml of methylene chloride and 0.5 ml of triethylamine, and cooled to 0° C. After slowly adding 0.1 M ethyl chloroformate in methylene chloride, the mixture is left to stir at 0° C. for 10 minutes. The reaction solution is then poured on to a mixture of 20 ml of saturated sodium chloride solution and 10 ml of ethyl acetate. The organic phase is washed once more with aqueous NaCl solution, filtered through a little silica gel and concentrated. The residue is separated by chromatography on silica gel (ethyl acetate/n-heptane=1/3 to 2/1). This affords 70 mg (27% yield) of carbonate 71 as a colorless solid and, as well as reactant 15 (20%), also several by-products. C₂₄H₂₇ClF₂O₇ (500.93), MS (ESI⁺) 518.14 (M+NH₄⁺).

### Example 20 and 5

### Compound 72 and 37

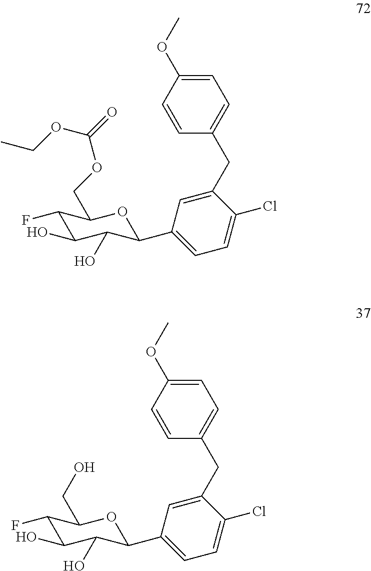

The C-glycosides 72 and 37, which has already been described via another synthesis route, are prepared analogously to the method for the synthesis of example 8 and 9, proceeding from 4-bromo-1-chloro-(4-methoxybenzyl)benzene and lactone 53, with similar yields.

MS for compound 72: C₂₃H₂₆ClFO₇ (468.91), MS (ESI⁺) 486.31 (M+NH₄).

### Example 21 and 22

### Compound 73 and 74

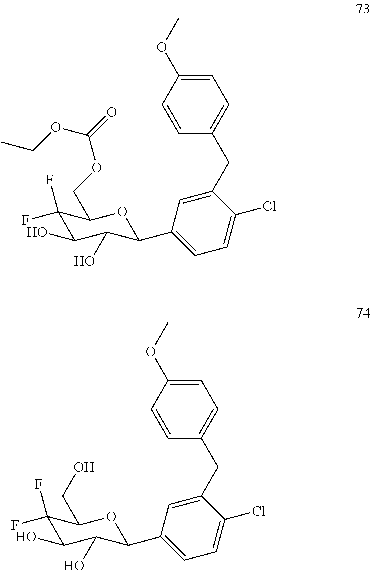

The C-glycosides 73 and 74 are prepared analogous to the method for the synthesis of example 10 and 11, proceeding from 4-bromo-1-chloro-(4-methoxybenzyl)benzene and lactone 53, with similar yields.

MS for compound 73: C₂₃H₂₅ClF₂O₇ (486.90), MS (ESI⁺) 469.15 (M−H₂O+H⁺).

MS for compound 74: C₂₀H₂₁ClF₂O₅ (414.18), MS (ESI⁺) 432.18 (M+NH₄⁺).

### Example 23

### Compound 75

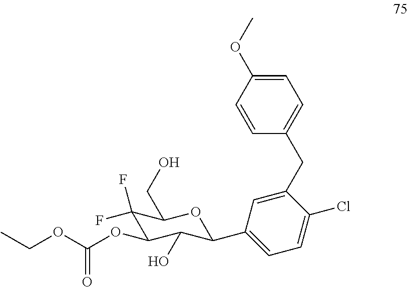

The 3-carbonate 75 is prepared analogously to the compound 71, proceeding from example 22. C₂₃H₂₅ClF₂O₇ (486.90), MS (ESI⁺) 504.32 (M+NH₄⁺).

### Synthesis of Compound 76

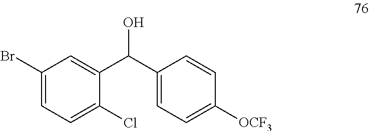

The bromide 76 is, analogously to the method for the synthesis of bromide 47, prepared proceeding from 4-bromo-1-chloro-2-iodobenzene and p-trifluoromethoxy-benzaldehyde, with similar yields.

### Synthesis of Compound 77

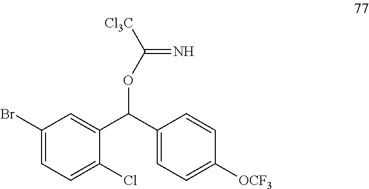

The benzyl alcohol 76 cannot be deoxygenated directly. It is therefore successfully deoxygenated by a newly devised method, via an activation by a trichloroacetimidate. 3.0 g (7.9 mmol) of benzyl alcohol 76 are dissolved in 40 ml of methylene chloride and 10 ml of trichloroacetonitrile and stirred with 700 mg of sodium hydride (55% in paraffin oil) at room temperature for 20 minutes. The mixture is then filtered through a little silica gel and concentrated. The residue is separated by chromatography on silica gel (ethyl acetate/n-heptane=1/6 to 1/4). This affords 4.0 g (97% yield) of product 77 as a colorless oil. C₁₆H₉BrCl₄F₃NO₂ (525.97), MS (ESI⁺) 364.94 (M−Cl₃CCONH₂+H⁺).

### Synthesis of Compound 78

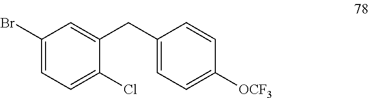

4.0 g (7.6 mmol) of compound 77 are dissolved in 25 ml of acetonitrile, 25 ml of methylene chloride and 5 ml of triethylsilane, and cooled to −40° C. After adding 2.5 ml of boron trifluoride etherate, the mixture is stirred at −40° C. for 30 minutes. The reaction solution is then added to a mixture of 30 ml of water and 50 ml of ethyl acetate. The organic phase is washed once more with aqueous NaCl solution, filtered through a little silica gel and concentrated. The residue is separated by chromatography on silica gel (ethyl acetate/n-heptane=0/1 to 1/6). This affords 1.3 g (47% yield) of compound 78 as a colorless oil.

### Example 24 and 25

### Compound 79 and 80

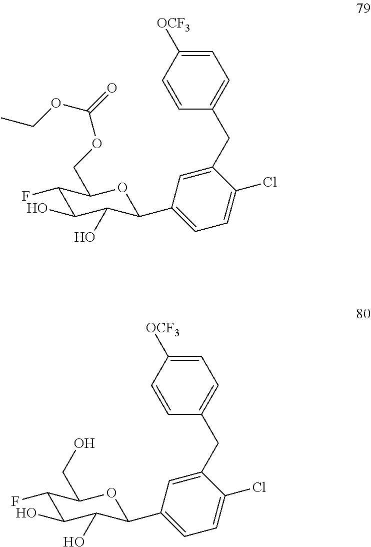

The C-glycosides 79 and 80 are prepared analogously to the method for the synthesis of example 8 and 9, proceeding from 4-bromo-1-chloro-(4-trifluoromethoxy-benzyl)benzene 78 and lactone 53, with similar yields.

MS for compound 79: C₂₃H₂₃ClF₄O₇ (522.88), MS (ESI⁺) 540.42 (M+NH₄⁺).

MS for compound 80: C₂₀H₁₉ClF₄O₅ (450.82), MS (ESI⁺) 468.06 (M+NH₄⁺).

### Example 26 and 27

### Compound 81 and 82

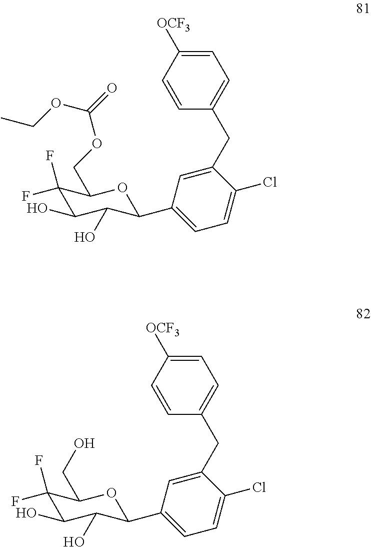

The C-glycosides 81 and 82 are prepared analogously to the method for the synthesis of example 10 and 11, proceeding from 4-bromo-1-chloro-(4-trifluoro-methoxybenzyl)benzene 78 and lactone 53, with similar yields.

MS for compound 81: C₂₃H₂₂ClF₅O₇ (540.87), MS (ESI⁺) 523.07 (M−H₂O+Hⁱ).

MS for compound 82: C₂₀H₁₈ClF₅O₅ (468.81), MS (ESI⁺) 486.05 (M+NH₄⁺).

### Synthesis of Compound 83

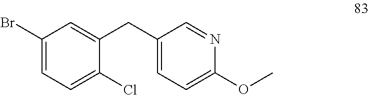

The bromide 83 is, analogously to the method for the synthesis of bromide 78, prepared proceeding from 4-bromo-1-chloro-2-iodobenzene and 6-methoxypyridine-3-carbaldehyde, with similar yields. C₁₃H₁₁BrClNO (312.60), MS (ESI⁺) 313.94 (M+H⁺).

### Example 28 and 29

### Compound 84 and 85

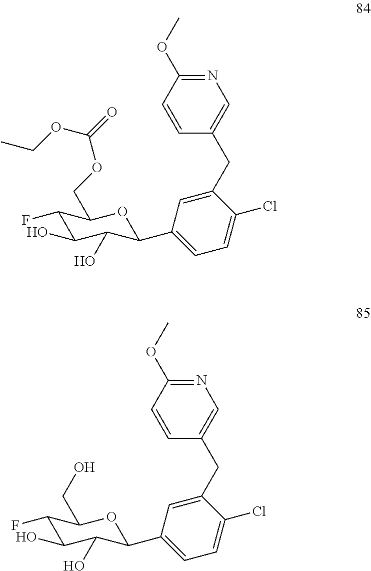

The C-glycosides 84 and 85 are prepared analogously to the method for the synthesis of example 8 and 9, proceeding from bromide 83 and lactone 53, with similar yields.

MS for compound 84: C₂₂H₂₅ClFNO₇ (469.90), MS (ESI⁺) 470.02 (M+H⁺).

MS for compound 85: C₁₉H₂₁ClFNO₅ (397.83), MS (ESI⁺) 398.08 (M+H⁺).

### Example 30

### Compound 86

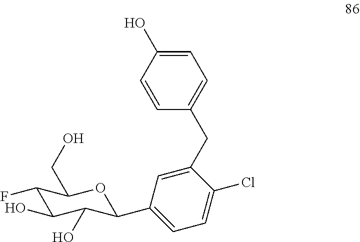

The C-glycoside 86 is prepared analogously to the method for the synthesis of example 1, proceeding from 2-(4-benzyloxybenzyl)-4-bromo-1-chlorobenzene, with similar yields. C₁₉H₁₉ClF₂O₅ (400.81), MS (ESI⁺) 383.10 (M−H₂O+H⁺).

### Example 31

### Compound 87

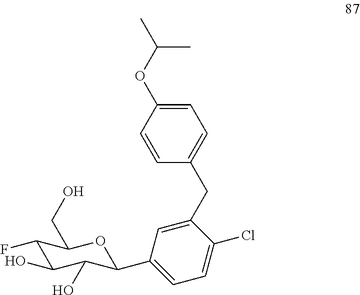

90 mg (0.22 mmol) of phenol 86 are dissolved in 2 ml of DMF and 1 ml of 2-iodopropane. After adding 300 mg of potassium carbonate, the mixture is left to stir at room temperature for 20 hours. The reaction solution is then poured on to a mixture of 10 ml of water and 10 ml of ethyl acetate. The organic phase is washed twice more with aqueous NaCl solution, filtered through a little silica gel and concentrated. The residue is separated by chromatography on silica gel (ethyl acetate/n-heptane=1/1 to 1/0). This affords 93 mg (98% yield) of product 87 as a colorless solid. C₂₂H₂₅ClF₂O₅ (442.89), MS (ESI⁺) 460.21 (M+NH₄).

### Example 32

### Compound 88

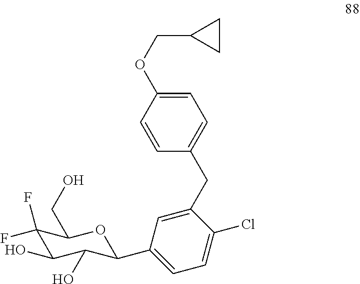

Analogously to example 31, compound 88 is obtained proceeding from iodomethylcyclopropane and phenol 86. C₂₃H₂₅ClF₂O₅ (454.90), MS (ESI⁺) 472.36 (M+NH₄⁺).

### Example 33

### Compound 89

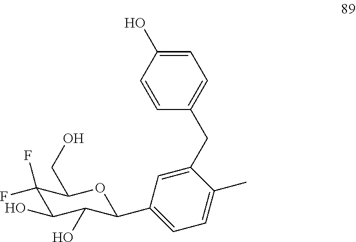

The C-glycoside 89 is prepared analogously to the method for the synthesis of example 1, proceeding from 2-(4-benzyloxybenzyl)-4-bromo-1-methylbenzene, with similar yields. C₂₀H₂₂F₂O₅ (380.39), MS (ESI⁺) 398.29 (M+NH₄).

### Example 34

### Compound 90

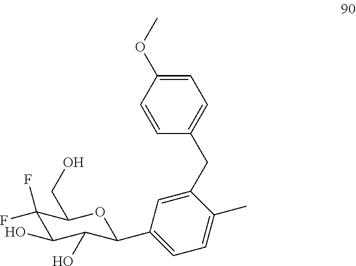

Analogously to example 31, compound 90 is obtained proceeding from iodomethane and phenol 89. C₂₁H₂₄F₂O₅ (398.29), MS (ESI⁺) 412.27 (M+NH₄).

### Synthesis of Compound 91

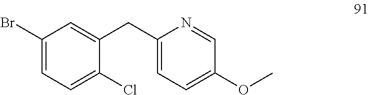

The bromide 91 is, analogously to the method for the synthesis of bromide 78, prepared proceeding from 4-bromo-1-chloro-2-iodobenzene and 5-methoxypyridine-2-carbaldehyde, with similar yields. C₁₃H₁₁BrClNO (312.60), MS (ESI⁺) 313.94 (M+H⁺).

### Example 35 and 36

### Compound 92 and 93

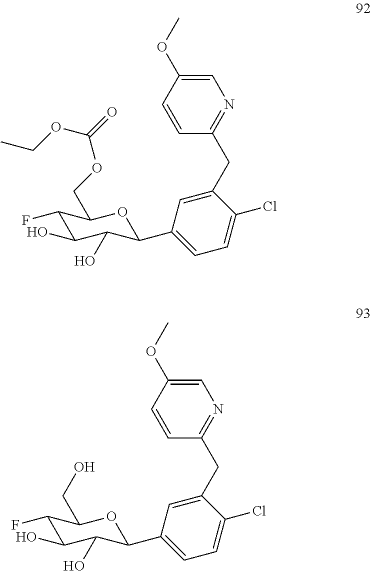

The C-glycosides 92 and 93 are prepared analogously to the method for the synthesis of examples 8 and 9, proceeding from bromide 91 and lactone 53, with similar yields.

MS for compound 92: C₂₂H₂₅ClFNO₇ (469.90), MS (ESI⁺) 470.02 (M+H⁺).

MS for compound 93: C₁₉H₂₁ClFNO₅ (397.83), MS (ESI⁺) 398.08 (M+H⁺).

### Example 37 and 38

### Compound 94 and 95

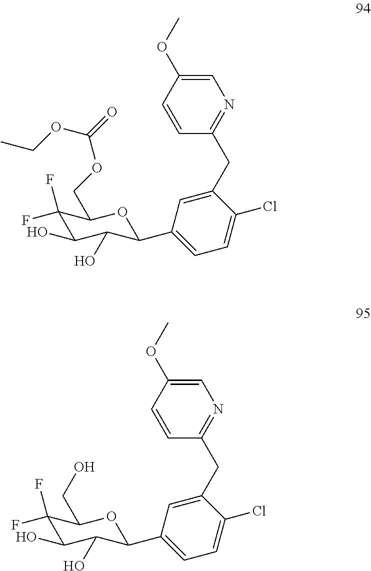

The C-glycosides 94 and 95 are prepared analogously to the method for the synthesis of example 10 and 11, proceeding from bromide 91 and lactone 53, with similar yields.

MS for compound 94: C₂₂H₂₅ClF₂NO₇ (487.89), MS (ESI⁺) 488.06 (M+H⁺).

MS for compound 95: C₁₉H₂₁ClF₂NO₅ (415.82), MS (ESI⁺) 416.06 (M+H⁺).

### Synthesis of Compound 96

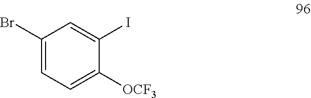

10.74 g (93.75 mmol) of tert-butyl nitrite and 28.5 g (150 mmol) of copper iodide are suspended in 270 ml of acetonitrile and heated to 60° C. A solution of 15 g (62.5 mmol) of 5-bromo-2-trifluoromethoxyaniline in 130 ml of acetonitrile is slowly added dropwise to this suspension and the mixture is left to stir at 60° C. for another hour. The reaction solution is then poured on to a mixture of 250 ml of 2 N aqueous HCl and 250 ml of ethyl acetate. The organic phase is washed twice more with aqueous NaCl solution, filtered through a little silica gel and concentrated. The residue is separated by chromatography on silica gel (ethyl acetate/n-heptane=1/18). This affords 12.2 g (52% yield) of product 96 as a colorless oil.

### Synthesis of Compound 97

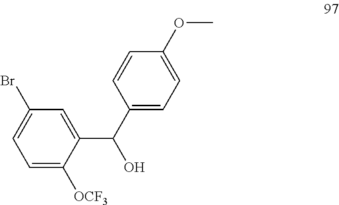

6.0 g (16.4 mmol) of iodide 96 are dissolved in 50 ml of dry tetrahydrofuran (THF) and cooled to −78° C. with an acetone/dry ice mixture under an argon atmosphere. After adding 8.8 ml of a 2.6 molar n-butyllithium solution in toluene (22.9 mmol), the reaction solution is stirred at −78° C. for 20 minutes. A solution of 3.2 g (22.9 mmol) of anisaldehyde in 20 ml of THF is then added dropwise to the reaction solution and the mixture is stirred at −78° C. for one hour. The solution is poured on to 100 ml of 10% ammonium chloride solution and 100 ml of ethyl acetate. The organic phase is washed once more with aqueous NaCl solution, filtered through a little silica gel and concentrated. This affords 8 g of crude product 97 as a colorless oil.

### Synthesis of Compound 98

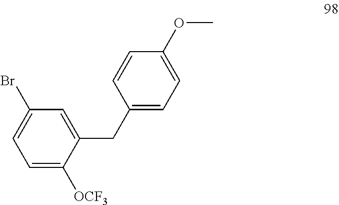

8 g of crude product 97 are dissolved in 100 ml of acetonitrile, 100 ml of methylene chloride and 15 ml of triethylsilane and cooled to −40° C. with an acetone/dry ice mixture under an argon atmosphere. After adding 8 ml of boron trifluoride etherate, the reaction solution is left to stir at −40° C. for 30 minutes. The reaction mixture is then poured on to a mixture of 100 ml of saturated sodium chloride solution and 100 ml of ethyl acetate. The organic phase is washed once more with aqueous NaCl solution, filtered through a little silica gel and concentrated. The residue is separated by chromatography on silica gel (ethyl acetate/n-heptane=1/6 to 1/4). This affords 1.8 g (31% yield over 2 stages) of product 98 as a colorless oil.

### Example 39 and 40

### Compound 99 and 100

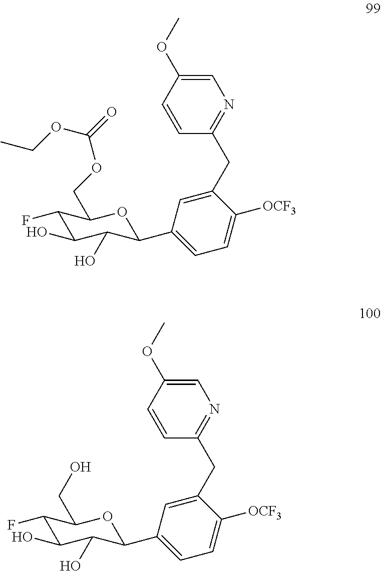

The C-glycosides 99 and 100 are prepared analogously to the method for the synthesis of example 8 and 9, proceeding from bromide 98 and lactone 53, with similar yields.

MS for compound 99: C₂₄H₂₆F₄O₈ (518.46), MS (ESI⁺) 541.18 (M+Na⁺).

MS for compound 100: C₂₁H₂₂F₄O₆ (446.40), MS (ESI⁺) 464.20 (M+NH₄⁺).

### Example 41 and 42

### Compound 101 and 102

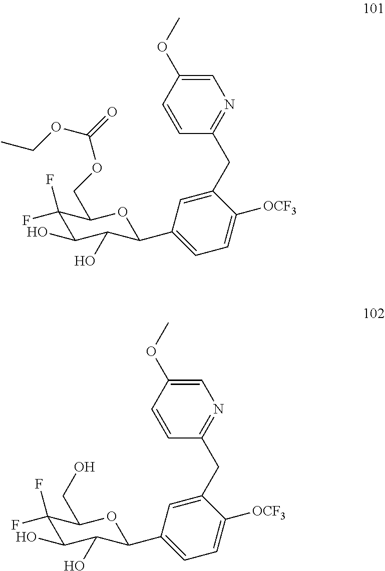

The C-glycosides 101 and 102 are prepared analogously to the method for the synthesis of example 10 and 11, proceeding from bromide 98 and lactone 53, with similar yields.

MS for compound 101: C₂₄H₂₅F₅O₈ (536.45), MS (ESI⁺) 537.07 (M+H⁺).

MS for compound 102: C₂₁H₂₁F₅O₆ (464.39), MS (ESI⁺) 482.07 (M+NH₄⁺).

### Synthesis of Compound 103

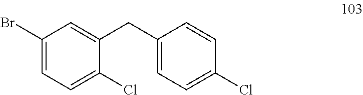

The bromide 103 is, analogously to the method for the synthesis of bromide 78, prepared proceeding from 4-bromo-1-chloro-2-iodobenzene and p-chlorobenzaldehyde, with similar yields. C₁₃H₉BrCl₂ (316.03), MS (ESI⁺) 314.93 (M+H⁴).

### Example 43 and 44

### Compound 104 and 105

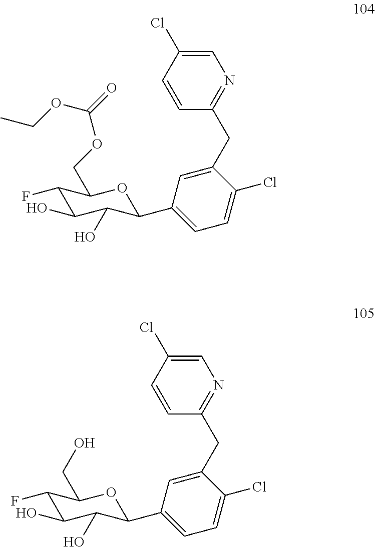

The C-glycosides 104 and 105 are prepared analogously to the method for the synthesis of example 8 and 9, proceeding from bromide 103 and lactone 53, with similar yields.

MS for compound 104: C₂₂H₂₃Cl₂FO₆ (473.33), MS (ESI⁺) 495.12 (M+NH₄⁺).

MS for compound 105: C₁₉H_{19I}Cl₂FO₄ (401.27), MS (ESI⁻) 446.07 (M+HCOO⁻).

### Example 45 and 46

### Compound 106 and 107

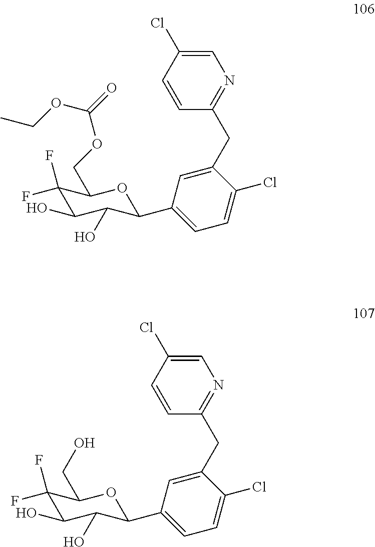

The C-glycosides 106 and 107 are prepared analogously to the method for the synthesis of example 10 and 11, proceeding from bromide 103 and lactone 53, with similar yields.

MS for compound 106: C₂₂H₂₂Cl₂F₂O₆ (491.32), MS (ESI⁺) 513.10 (M+Na⁺).

MS for compound 107: C₁₉H₁₈Cl₂F₂O₄ (419.26), MS (ESI⁺) 437.22 (M+NH₄⁺).

### Synthesis of Compound 110

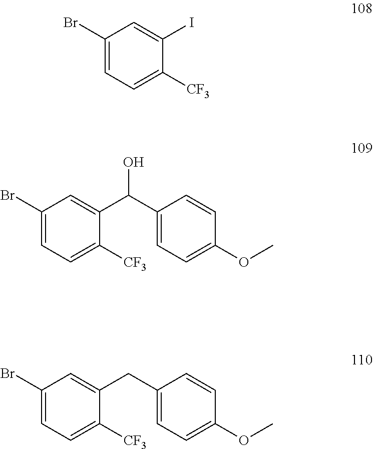

Proceeding from 5-bromo-2-trifluoromethylaniline and anisaldehyde, compound 110 is prepared in similar yields to compound 98, via the same reaction sequence.

### Example 47 and 48

### Compound 111 and 112

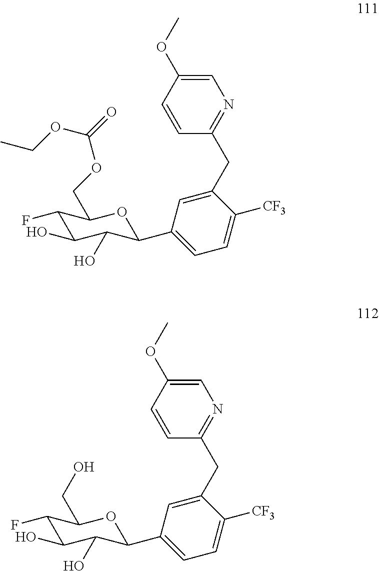

The C-glycosides 111 and 112 are prepared analogously to the method for the synthesis of example 8 and 9, proceeding from bromide 110 and lactone 53, with similar yields.

MS for compound III: C₂₄H₂₆F₄O₇ (502.46), MS (ESI⁺) 503.10 (M+H⁺).

MS for compound 112: C₂₁H₂₂F₄O₅ (430.40), MS (ESI⁺) 448.09 (M+NH₄).

### Example 49 and 50

### Compound 113 and 114

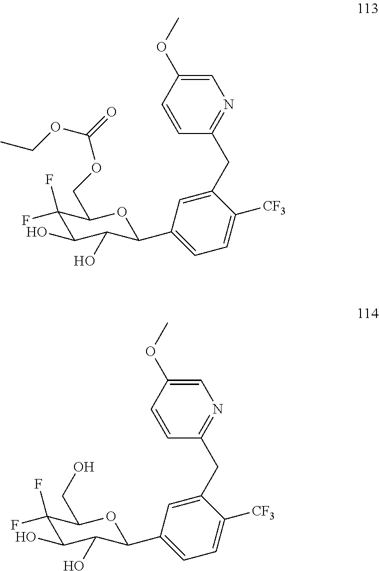

The C-glycosides 113 and 114 are prepared analogously to the method for the synthesis of example 10 and 11, proceeding from bromide 110 and lactone 53, with similar yields.

MS for compound 113: C₂₄H₂₅F₅O₇ (520.45), MS (ESI⁺) 521.12 (M+H⁺).

MS for compound 114: C₂₁H₂₁F₅O₅ (448.39), MS (ESI⁺) 470.87 (M+Na⁺).

### Synthesis of Compound 116

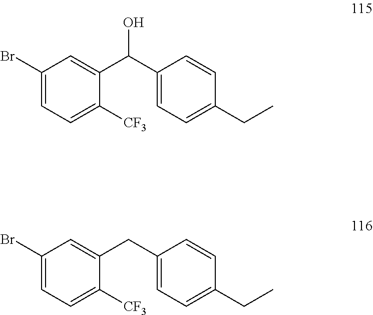

Proceeding from iodide 108 and 4-ethylbenzaldehyde, compound 116 is prepared in similar yields to compound 98, via the same reaction sequence.

### Example 51 and 52

### Compound 117 and 118

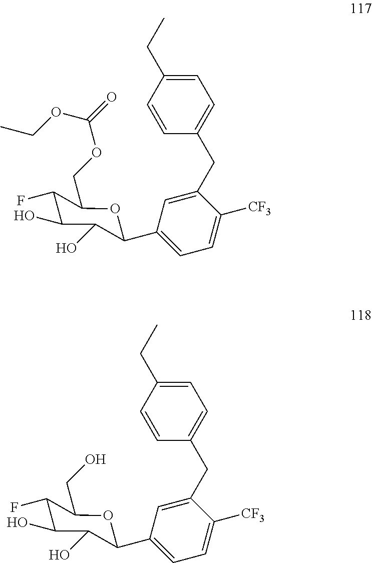

The C-glycosides 117 and 118 are prepared analogously to the method for the synthesis of example 8 and 9, proceeding from bromide 116 and lactone 53, with similar yields.

MS for compound 117: C₂₅H₂₈F₄O₆ (500.49), MS (ESI⁺) 501.28 (M+H⁺).

MS for compound 118: C₂₂H₂₄F₄O₄ (428.43), MS (ESI⁺) 446.16 (M+NH₄⁺).

### Example 53 and 54

### Compound 119 and 120

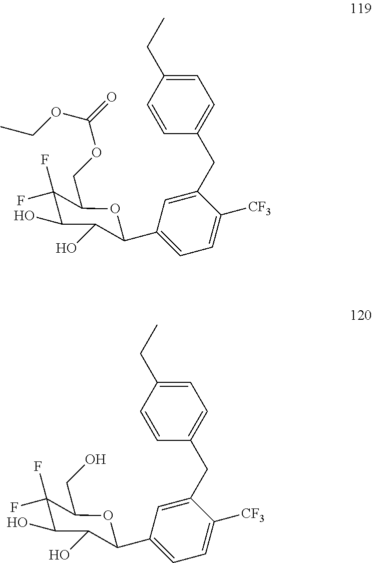

The C-glycosides 119 and 120 are prepared analogously to the method for the synthesis of example 10 and 11, proceeding from bromide 116 and lactone 53, with similar yields.

MS for compound 119: C₂₅H₂₇F₅O₆ (518.48), MS (ESI⁺) 536.16 (M+NH₄). MS for compound 120: C₂₁H₂₁F₅O₅ (446.42), MS (ESI⁺) 464.08 (M+NH₄⁺).

### Example 55 and 56

### Compound 121 and 122

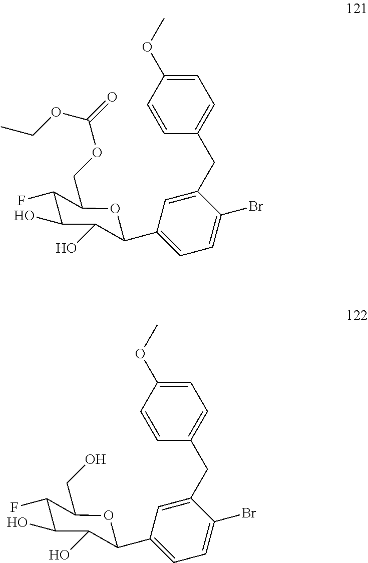

The C-glycosides 121 and 122 are prepared analogously to the method for the synthesis of example 8 and 9, proceeding from 1-bromo-4-iodo-2-(4-methoxybenzyl)benzene and lactone 53, with similar yields.

MS for compound 121: C₂₃H₂₆BrFO₇ (513.36), MS (ESI⁺) 514.96 (M+H⁺).

MS for compound 122: C₂₀H₂₂BrFO₅ (441.30), MS (ESI⁺) 882.92 (2×M+H⁺).

### Example 57 and 58

### Compound 123 and 124

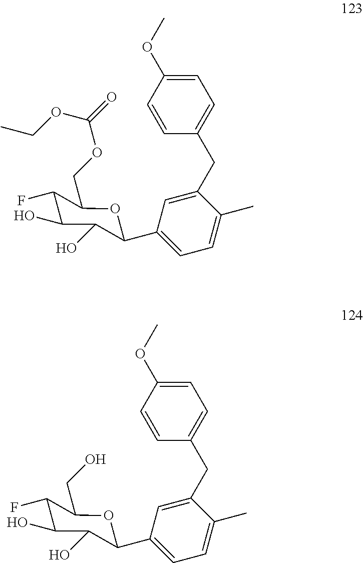

The C-glycosides 123 and 124 are prepared analogously to the method for the synthesis of example 8 and 9, proceeding from 4-bromo-2-(4-methoxybenzyl)toluene and lactone 53, with similar yields.

MS for compound 123: C₂₄H₂₉FO₇ (448.49), MS (ESI⁺) 471.14 (M+Na⁺).

MS for compound 124: C₂₁H₂₅FO₅ (376.43), MS (ESI⁺) 399.24 (M+Na⁺).

### Example 59

### Compound 125

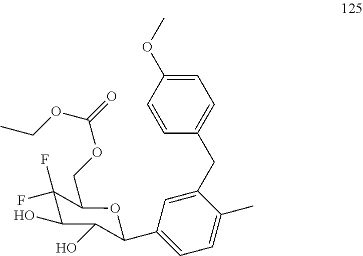

The C-glycoside 125 is prepared analogously to the method for the synthesis of example 10, proceeding from 4-bromo-2-(4-methoxybenzyl)toluene and lactone 53, with similar yield.

MS for compound 125: C₂₄H₂₈F₂O₇ (466.48), MS (ESI⁺) 467.15 (M+H⁺).

### Example 60 and 61

### Compound 126 and 127

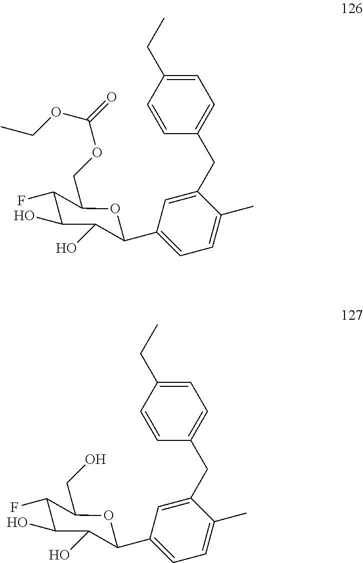

The C-glycosides 126 and 127 are prepared analogously to the method for the synthesis of example 8 and 9, proceeding from 4-bromo-2-(4-methylbenzyl)toluene and lactone 53, with similar yields.

MS for compound 126: C₂₅H₃₁FO₆ (446.52), MS (ESI⁺) 464.33 (M+NH₄⁺).

MS for compound 127: C₂₂H₂₇FO₄ (374.46), MS (ESI⁺) 392.30 (M+NH₄⁺).

### Example 62 and 63

### Compound 128 and 129

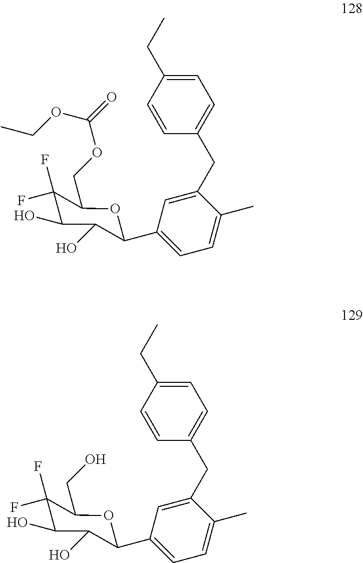

The C-glycosides 128 and 129 are prepared analogously to the method for the synthesis of example 10 and 11, proceeding from 4-bromo-2-(4-methylbenzyl)toluene and lactone 53, with similar yields.

MS for compound 128: C₂₅H₃₀F₂O₆ (464.51), MS (ESI⁺) 482.27 (M+NH₄⁺).

MS for compound 129: C₂₂H₂₆F₂O₄ (392.45), MS (ESI⁺) 410.26 (M+NH₄⁺).

Further compounds which have been prepared according to the above-described processes.

### Example 64

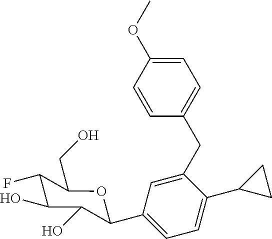

### Example 65

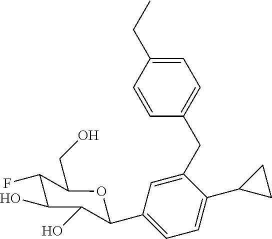

### Example 66

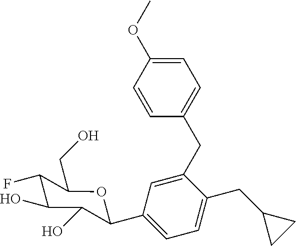

### Example 67

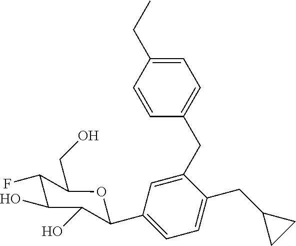

1. A compound of the formula (I) 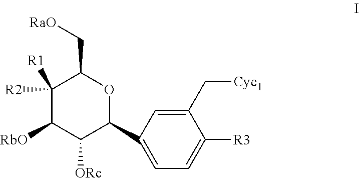
in which
Ra, Rb, Rc are each independently H, —COO—(C₁-C₆)-alkyl;
R1 and R2 are each F or
R1 is H and R2 is F;
R3 is hydrogen, F, Cl, Br, CF₃, OCF₃, CN, methyl, ethyl, methoxy, ethoxy, cyclopropyl, CH₂-cyclopropyl;
Cyc1 is 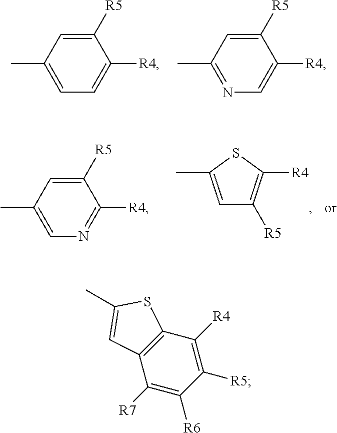
R4, R5, R6, R7 are each independently hydrogen, F, Cl, Br, I, OH, CF₃, NO₂, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, O—(C₁-C₆)-alkyl, HO—(C₁-C₆)-alkylene, (C₁-C₆)-alkylene-O—(C₁-C₆)-alkyl, where one, more than one or all hydrogen(s) in the alkyl, alkenyl, alkynyl or O-alkyl radicals may be replaced by fluorine;
SO₂—NH₂, SO₂NH(C₁-C₆)-alkyl, SO₂N[(C₁-C₆)-alkyl]₂, S—(C₁-C₆)-alkyl, SCF₃, SO—(C₁-C₆)-alkyl, SO₂—(C₁-C₆)-alkyl, NH₂;
and pharmaceutically compatible salts thereof;
excluding the compound where
R1=H, R2=F, R3=methyl and Cyc1-R4=4—OCH₃-phenyl.

2. The compound of claim 1 wherein
Cyc1 is

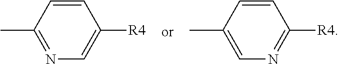

3. The compound of claim 1 wherein
Cyc1 is

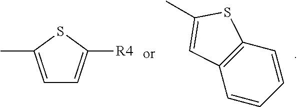

4. The compound of claim 2 or 3 wherein
R1 and R2 are each F.

5. The compound of claim 1 wherein
Ra is —COO—(C₁-C₆)-alkyl; and
Rb, Rc are each hydrogen.

6. A medicament comprising one or more of the compounds as claimed in claim 1 and one or more active ingredients which lower blood sugar.

7. A method of treating type 1 and type 2 diabetes comprising administering to a patient in need thereof a therapeutically effective amount of a compound of claim 1.

8. A method of lowering blood sugar comprising administering to a patient in need thereof a therapeutically effective amount of a compound of claim 1.

9. A medicament comprising one or more compounds as claimed in claim 1 and at least one further active ingredient which lowers blood sugar for the treatment of type 1 and type 2 diabetes.

10. A process for producing a medicament comprising one or more of the compounds of claim 1, which comprises mixing the active ingredient with a pharmaceutically suitable carrier and converting this mixture to a form suitable for administration.
